SEQUENCE LISTING (105 sequences)

SEQ ID NO: 1 - protein, 8 aa, Homo sapiens
ITLRVRSH

SEQ ID NO: 2 - protein, 7 aa, Homo sapiens
EERLRSY

SEQ ID NO: 3 - protein, 7 aa, Homo sapiens
YERVRWY

SEQ ID NO: 4 - protein, 7 aa, Homo sapiens
EERLRSY

SEQ ID NO: 5 - protein, 7 aa, Homo sapiens
AERIRSI

SEQ ID NO: 6 - protein, 7 aa, Homo sapiens
EERLRSY

SEQ ID NO: 7 - protein, 12 aa, Homo sapiens
TVHGDLRLRSLP

SEQ ID NO: 8 - protein, 12 aa, Homo sapiens
TNDIGLRQRSHS

SEQ ID NO: 9 - protein, 12 aa, Homo sapiens
SPLLDGQRERSY

SEQ ID NO: 10 - protein, 12 aa, Homo sapiens
YDLPMRSRSYPG

SEQ ID NO: 11 - protein, 4 aa, Homo sapiens
  Xaa may be any amino acid
RXRS

SEQ ID NO: 12 - protein, 15 aa, Homo sapiens
KGSDQAIITLRVRSH

SEQ ID NO: 13 - protein, 5 aa, Homo sapiens
  Xaa may be any amino acid
RXRSH

SEQ ID NO: 14 - protein, 269 aa, Homo sapiens
MAAALFVLLGFALLGTHGASGAAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 15 - protein, 7 aa, Homo sapiens
PERILSI

SEQ ID NO: 16 - protein, 9 aa, Homo sapiens
GGSRARNLP

SEQ ID NO: 17 - protein, 463 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 18 - protein, 570 aa, Homo sapiens
EVKLEESGGGLVQPGGSMKLSCVASGFTFSNYWMNWVRQSPEKGLEWVAEIRLKSNNYATHYAESVKGRFTISRDDSKSSVYLQMNNLRAEDTGIYYCTDYDAYWGQGTLVTVSAESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 19 - protein, 206 aa, Homo sapiens
KFLLVSAGDRVTITCKASQSVSNDVAWYQQKPGQSPKLLIYYASNRYTGVPDRFTGSGYGTDFTFTISTVQAEDLAVYFCQQDYSSPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 20 - protein, 12 aa, Homo sapiens
SLAPLWYYSRHG

SEQ ID NO: 21 - protein, 12 aa, Homo sapiens
HTPETAPLPATV

SEQ ID NO: 22 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 23 - protein, 205 aa, Homo sapiens
  Xaa may be any amino acid
GLLKPSETLSLTCAVYGGSFSGYYWSWIRQPPGKGLEWIGEINHSGSTNYNPSLKSRVTISVDTSKNQFSLKLSSVTAADTAVYYCARGTTEYYYYYYGMDVWGQGTTVTVSSGSASAPTLFPLVSCENSPSDTSSVAVGCLAQDFLPDXITFSWKYKNNSDISSTRGFPSVLRGGKYAATSQVLLPSKDVMQGTDEHVVTGSKE

SEQ ID NO: 24 - protein, 148 aa, Homo sapiens
LSLPVTPGEPASISCRSSQSLLHSNGYNYLDWYLQKPGQSPQLLIYLGSNRASGVPDRFSGSGSGTDFTLKISRVEAEDVGIYYCMQTRQTPRTFGQGTKVEIKRTVAAPSVFIFPPSDEQLKSGTASVVCLLNNFYPREAKEHQKSP

SEQ ID NO: 25 - protein, 197 aa, Homo sapiens
LVKPSETLSLTCTVSGGSISSYYWNWIRQPPGKGLEWIGYIYYSGSTNYNPSLKSRVTISVDTSKNQFSLKLSSVTAADTAVYYCARDRGVGATGFDYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHKVC

SEQ ID NO: 26 - protein, 147 aa, Homo sapiens
SQSPSSLSASVGERVTITCRASQGIRDELGWYQQKPGKAPKRLIYVASSLQSGVPSRFSGSGSGTEFTLTISSLQPEDFATYYCLQHNGYPRTFGQGTKVEIKRTVAAPSVFIFPPSDEQLKSGTASVVCLLNNFYPREAKEHQKSP

SEQ ID NO: 27 - protein, 203 aa, Homo sapiens
KKPGASVKVSCKASGYTFTSYDINWVRQATGQGLEWMGWMNPNSGNTGYAQKFQGRVTMNRNTSISTAYMELSSLRSEDTAVYYCARGGHGGSYFYSYYGMDVWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCK

SEQ ID NO: 28 - protein, 149 aa, Homo sapiens
HSLAVSLGERATINCKSSQSVLYSFNNKNYLAWYQQKPGQPPKLLIYWASTRESGVPDRFGGSGSGTDFTLTISSLQAEDVAVYYCQQYYSTPRTFGQGTKVEIKRTVAAPSVFIFPPSDEQLKSGTASVVCLLNNFYPREAKEHQKSP

SEQ ID NO: 29 - protein, 199 aa, Homo sapiens
EVKKPGASVKVSCKASGYTFTSYDINWVRQATGQGLEWMGWMNPNSGNTGYAQKFQGRVTMTRNTSISTAYMELSSLRSEDTAVYYCAREEWLVRYYGMDVWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHKV

SEQ ID NO: 30 - protein, 147 aa, Homo sapiens
  Xaa may be any amino acid
GQSPSSLSASVGDRVTITCRASQDIRDNLGWYQQKPGKAPKRLIYAASNLQSGVPSRFSGSGSGTEFTLTISSLQPEDFATYYCLQYKTYPWTFGQGTKVEIKRTVAAPSVFIFPPSDEQLKSGTASVVCLLNNFYPREXKEHQKSP

SEQ ID NO: 31 - protein, 202 aa, Homo sapiens
  Xaa may be any amino acid
KLPETLSLTCAVYGGSFSGYYWSWIRQPPGKGLEWIGEINHSGSTNYNPSLKSRVTISVDTSKNQFSLKLSSVTAADTAVYYCARGAAEYYYYYYGMDVWGQGTTVTVSSGSASAPTLFPLVSCENSPSDTSSVAVGCLAQDFLPDXITFXWKYKNNSDISSTRGFPSVLRGGKYAATSQVLLPSKDVMQGTDEHVVTGSKE

SEQ ID NO: 32 - protein, 143 aa, Homo sapiens
MPVTPGEPASISCRSSQSLLHSNGYNYLDWYLQKPGQSPQLLIYLGSNRASGVPDRFSGSGSGTDFTLKISRVEAEDVGIYYCMQSLQIPRLFGPGTKVDIKRTVAAPSVFIFPPSDEQLKSGTASVVCLLSNFYPREAKVQW

SEQ ID NO: 33 - protein, 190 aa, Homo sapiens
SETLSLTCAVYGGSFSGYYWSWIRQPPGKGLEWIGEINHSGSTNYNPSLKSRVTISVDTSKNQFSLKLSSVTAADTAVYYCARGGTTVTFDAFDIWGQGTMVTVSSGSASAPTLFPLVSCENSPSDTSSVAVGCLAQDFLPDSITFSWKYKNNSDISSTRGFPSVLRGGKYAATSQVLLPSKDVMQGTDE

SEQ ID NO: 34 - protein, 147 aa, Homo sapiens
LAVSLGERATINCKSSQSVLYSFNNKNYLAWYQQKPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVI

SEQ ID NO: 35 - protein, 149 aa, Homo sapiens
NPQTTLTLTCTFSGFSLITRGVGVDWIRQPPGKALQWLALIYWNDDKRYSPSLKSRLTITKDTSKNQVVLTMTNMDPVDTATYYCAHHFFDSSGYYPFDSWGQGTLVSVSSASTKGPSVFPLAPCSRSTSESTAALGCLVKDYFPEPVT

SEQ ID NO: 36 - protein, 148 aa, Homo sapiens
VTQSPLSLSVTPGQPASISCKSSQSLLHSDGKTYLYWYLQKPGQPPQLLIYEAFNRFSGVPDRFSGSGSGTDFTLKISRVEAEDVGLYYCMQSIELPFTFGGGTKVEIKRTVAAPSVFIFPPSDEQLKSGTASVVCLLNNFYPRKERV

SEQ ID NO: 37 - protein, 173 aa, Homo sapiens
GEGLVKPGGSLRLSCAASGFTFSSYSMNWVRQAPGKGLEWVSSISSSSSYIYYADSVKGRFTISRDNAKNSLYLQMNSLRAEDTAVYYCARDSSGWYEDYFDYWGQGTLVTVSSASTKGPSVFPLAPCSRSTSESTAALGCLVKDYFPEPVTVSWNSGALTSGVHTFPAVLQS

SEQ ID NO: 38 - protein, 101 aa, Homo sapiens
LDIQLTQSPSSLSASVGDRVTITCRASQDISIYLAWFQQRPGKAPKSLIYAASSLQSGVPSKFSGSGSGTDFTLTISSLQPEDFATYYCQQYNSYPFTFGP

SEQ ID NO: 39 - protein, 159 aa, Homo sapiens
LTCTFSGFSLITRGVGVDWIRQPPGKALQWLALIYWNDDKRYSPSLKSRLTITKDTSKNQVVLTMTNMDPVDTATYYCAHHFFDSSGYYPFDSWGQGTLVSVSSASTKGPSVFPLAPCSRSTSESTAALGCLVKDYFPEPVTVSWNSGALTSGVHTFQL

SEQ ID NO: 40 - protein, 167 aa, Homo sapiens
GGGLVQPGGSLRLSCAASGFTFSSYAMSWVRQAPGKGLEWVSTISVSGITTYYVDSVKGRFTISRDNSKNILYLQMNSLRAEDTAVYYCAKRIFGVVWGQGTLVTVSSASTKGPSVFPLAPCSRSTSESTAALGCLVKDYFPEPVTVSWNLGALTSGVHTFPAVLQS

SEQ ID NO: 41 - protein, 164 aa, Homo sapiens
GIRLDIQLTQSPSSLSASVGDRVTITCRASQGISIYLAWFQQRPGKAPKSLIYAASSLQSGVPSKFSGSGSGTDFTLTISSLQPEDFATYYCQQYNSYPFTFGPGTKVDIKRTVAAPSVFIFPPSDEQLKSGTASVVCLLNNFYPREAKVQWKVDNALQSGKPN

SEQ ID NO: 42 - DNA, 35 bp, Homo sapiens
gactacgaattcttgtaggaccggcgaggaatagg

SEQ ID NO: 43 - DNA, 37 bp, Homo sapiens
gactacgggcccggtgagaacttggaatcttgcaagc

SEQ ID NO: 44 - DNA, 18 bp, Artificial Sequence
  Description of Artificial Sequence primer
gcagtctcctaaactgct

SEQ ID NO: 45 - DNA, 15 bp, Artificial Sequence
  Description of Artificial Sequence primer
acctgcaaggccagt

SEQ ID NO: 46 - DNA, 18 bp, Artificial Sequence
  Description of Artificial Sequence primer
cactcattcctgttgaag

SEQ ID NO: 47 - DNA, 500 bp, Homo sapiens
tcagaagaagtgaagtcaagatgaagaaccatttgcttttctggggagtcctggcggtttttattaaggctgttcatgtgaaagcccaagaagatgaaaggattgttcttgttgacaacaaatgtaagtgtgcccggattacttccaggatcatccgttcttccgaagatcctaatgaggacattgtggagagaaacatccgaattattgttcctctgaacaacagggagaatatctctgatcccacctcaccattgagaaccagatttgtgtaccatttgtctgacctctgtaaaaaatgtgatcctacagaagtggagctggataatcagatagttactgctacccagagcaatatctgtgatgaagacagtgctacagagacctgctacacttatgacagaaacaagtgctacacagctgtggtcccactcgtatatggtggtgagaccaaaatggtggaaacagccttaaccccagatgcctgctatcctgactaa

SEQ ID NO: 48 - DNA, 21 bp, Artificial Sequence
  Description of Artificial Sequence primer
gaattcagaagaagtgaagtc

SEQ ID NO: 49 - DNA, 24 bp, Artificial Sequence
  Description of Artificial Sequence primer
gtcgactatgcagtcagcaatgac

SEQ ID NO: 50 - DNA, 20 bp, Artificial Sequence
  Description of Artificial Sequence primer
tgcaggaatcagacccagtc

SEQ ID NO: 51 - DNA, 22 bp, Artificial Sequence
  Description of Artificial Sequence primer
gtcaggctggaactgaggagca

SEQ ID NO: 52 - DNA, 19 bp, Artificial Sequence
  Description of Artificial Sequence primer
tcatttggtgatcagcact

SEQ ID NO: 53 - DNA, 25 bp, Artificial Sequence
  Description of Artificial Sequence primer
gctagctgaggagacggtgaccagg

SEQ ID NO: 54 - DNA, 19 bp, Artificial Sequence
  Description of Artificial Sequence primer
tcatttggtgatcagcact

SEQ ID NO: 55 - DNA, 22 bp, Artificial Sequence
  Description of Artificial Sequence primer
ggatcctgaggagacggtgacg

SEQ ID NO: 56 - DNA, 24 bp, Artificial Sequence
  Description of Artificial Sequence primer
ggattagcatccgccccaaccctt

SEQ ID NO: 57 - DNA, 24 bp, Artificial Sequence
  Description of Artificial Sequence primer
gtcgacgcacacacagagcggcca

SEQ ID NO: 58 - DNA, 37 bp, Homo sapiens
gactacgaattcggaccggcgaggaataggaatcatg

SEQ ID NO: 59 - DNA, 43 bp, Homo sapiens
ggatggtgttggtagctagcacgcggagcgtgatgatggcctg

SEQ ID NO: 60 - DNA, 33 bp, Homo sapiens
gactacgaattcacgaggcgacatggcggcggc

SEQ ID NO: 61 - DNA, 43 bp, Homo sapiens
ggatggtgttggtagctagcacacgcagtgagatggtttcccg

SEQ ID NO: 62 - DNA, 617 bp, Homo sapiens
  “n” may be A, T, C, G, unknown or other
ggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgcngcggacacggctgtgtattactgtgcgagaggcactacggaatattactactactactacggtatggacgtctggggccaagggaccacggtcaccgtctcctcagggagtgcatccgccccaacccttttccccctcgtctcctgtgagaattccccgtcggatacgagcagcgtggccgttggctgcctcgcacaggacttccttcccgactycatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgacgggatccaaagagta

SEQ ID NO: 63 - DNA, 444 bp, Homo sapiens
ctctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttgggatttattactgcatgcagactcgacaaactcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagagcatcaaaagagtcca

SEQ ID NO: 64 - DNA, 593 bp, Homo sapiens
ctggtgaagccttcggagaccctgtccctcacctgcactgtctctggtggctccatcagtagttactactggaactggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagataggggagtgggagctactggttttgactactggggccagggaaccctggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacaaggtgtgcga

SEQ ID NO: 65 - DNA, 441 bp, Homo sapiens
agccagtctccatcctccctgtctgcatctgtaggagagagagtcaccatcacttgccgggcaagtcagggcattagagatgaattaggctggtatcagcagaaaccagggaaagcccctaagcgcctgatctatgttgcatccagtttgcaaagtggggtcccatcaaggttcagcggcagtggatctgggacagaattcactctcacaatcagcagcctgcagcctgaagattttgcaacttattactgtctacagcataatggttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagagcatcaaaagagtcca

SEQ ID NO: 66 - DNA, 610 bp, Homo sapiens
aagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaacaggaacacctccataagcacagcctacatggagctgagcagcctgagatctgaggacacggccgtgtattactgtgcgagagggggtcatggtgggagctacttctactcctaytacggtatggacgtctggggccaggggaccacggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaac

SEQ ID NO: 67 - DNA, 447 bp, Homo sapiens
cactccctggctgtgtctctgggcgagagggccaccatcaactgcaagtccagccagagtgttttatacagttttaacaataagaactacttagcttggtaccagcagaaaccaggacagcctcctaagctgctcatttactgggcatctacccgggaatccggggtccctgaccgattcggtggcagcgggtctgggacagatttcactctcaccatcagcagcctgcaggctgaagatgtggcagtttattactgtcagcaatattatagtactcctmggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagagcatcaaaagagtcca

SEQ ID NO: 68 - DNA, 599 bp, Homo sapiens
gaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagagaggagtggctggtacgttactacggtatggacgtctggggccaagggaccacggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacaaggtgtg

SEQ ID NO: 69 - DNA, 441 bp, Homo sapiens
ggccagtctccatcctccctgtctgcatctgtaggagacagagtcaccatcacttgccgggcaagtcaggacattagagataatttaggctggtatcagcagaaaccagggaaagcccctaagcgcctgatctatgctgcatccaatttgcaaagtggggtcccatcaaggttcagcggcagtggatctgggacagaattcactctcacaatcagcagcctgcagcctgaagattttgcaacttattactgtctacagtataaaacttacccgtggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggmcaaagagcatcaaaagagtcca

SEQ ID NO: 70 - DNA, 607 bp, Homo sapiens
aagcttccggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagaggggcagctgaatattactactactactacggtatggacgtctggggccaagggaccacggtcaccgtctcctcagggagtgcatccgccccaacccttttccccctcgtctcctgtgagaattccccgtcggatacgagcagcgtggccgttggctgcctcgcacaggacttccttcccgactycatcactttctyctggaaatacaagaacaactctgacatcagcagcacccggggcttcccatcagtcctgagagggggcaagtacgcagccacctcacaggtgctgctgccttccaaggacgtcatgcagggcacagacgaacacgtggtgacgggatccaaagagt

SEQ ID NO: 71 - DNA, 431 bp, Homo sapiens
atgcccgtcacccctggagagccggcctccatctcctgcaggtctagtcagagcctcctgcatagtaatggatacaactatttggactggtacctgcagaagccagggcagtctccacagctcctgatctatttgggttctaatcgggcctccggggtccctgacaggttcagtggcagtggatcaggcacagattttacactgaaaatcagcagagtggaggctgaggatgttgggatttattactgcatgcaaagtctacaaattccccggcttttcggccctgggaccaaagtggatatcaaacgaactgtggctgcaccatctgtcttcatcttcccgccatctgatgagcagttgaaatctggaactgcctctgttgtgtgcctgctgagtaacttctatcccagagaggccaaagtacagtggaa

SEQ ID NO: 72 - DNA, 570 bp, Homo sapiens
tcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagttctgtgaccgccgcggacacggctgtgtattactgtgcgagaggcgggactacagtaacttttgatgcttttgatatctggggccaagggacaatggtcaccgtctcttcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaa

SEQ ID NO: 73 - DNA, 441 bp, Homo sapiens
ctggctgtgtctctgggcgagagggccaccatcaactgcaagtccagccagagtgttttatacagttttaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtgatc

SEQ ID NO: 74 - DNA, 447 bp, Homo sapiens
aacccacagacgaccctcacgctgacctgcaccttctctgggttctcactcattacccgtggagtgggtgtggattggatccgtcagcccccaggaaaggccctgcagtggctcgcactcatttattggaatgatgataagcgctacagtccatctctgaagagcaggctcaccatcaccaaggacacctccaaaaaccaggtggtcctcacaatgaccaacatggaccctgtggacacagccacatattactgtgcacaccatttctttgatagtagtggttattacccttttgactcctggggccagggaaccctggtctccgtctcctcagcctccaccaagggcccatcggtcttccccctggcgccctgctccaggagcacctccgagagcacagcggccctgggctgcctggtcaaggactacttccccgaaccggtgacg

SEQ ID NO: 75 - DNA, 445 bp, Homo sapiens
gtgactcagtctccactctctctgtccgtcacccctggacagccggcctccatctcctgcaagtctagtcagagcctcctgcatagtgatggaaagacctatttgtattggtacctgcagaagccaggccagcctccacagctcctgatctatgaagctttcaaccggttctctggagtgccagataggttcagtggcagcgggtcagggacagatttcacactgaaaatcagccgggtggaggctgaggatgttggactttattattgcatgcaaagtatagagcttccgttcactttcggcggagggaccaaggtggagatcaaacgaactgtggctgcaccatctgtcttcatcttcccgccatctgatgagcagttgaaatctggaactgcctctgttgtgtgcctgctgaataacttctatcccagaaaagaaagagtcr

SEQ ID NO: 76 - DNA, 519 bp, Homo sapiens
ggggaaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagggatagcagtggctggtatgaggactactttgactactggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtca

SEQ ID NO: 77 - DNA, 303 bp, Homo sapiens
cttgacatccagctgacccagtctccgtcctcactgtctgcatctgtaggagacagagtcaccatcacttgtcgggcgagtcaggacattagcatttatttagcctggtttcagcagagaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacaatataatagttatccattcactttcgggccc

SEQ ID NO: 78 - DNA, 477 bp, Homo sapiens
ctgacctgcaccttctctgggttctcactcattacccgtggagtgggtgtggattggatccgtcagcccccaggaaaggccctgcagtggctcgcactcatttattggaatgatgataagcgctacagtccatctctgaagagcaggctcaccatcaccaaggacacctccaaaaaccaggtggtcctcacaatgaccaacatggaccctgtggacacagccacatattactgtgcacaccatttctttgatagtagtggttattacccttttgactcctggggccagggaaccctggtctccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttccagctg

SEQ ID NO: 79 - DNA, 503 bp, Homo sapiens
gggggaggcttggtacagcctggggggtccctgagactctcctgtgcagcctctggattcacttttagcagctatgccatgagctgggtccgccaggctccagggaaggggctggagtgggtctcaactattagtgttagtggtattaccacatactacgtagactccgtgaagggccggttcaccatctccagagacaattccaagaacattctgtatctgcaaatgaacagcctgagagccgaggacacggccgtatattactgtgcgaaacggatttttggagtggtctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaacttaggcgctctgaccagcggcgtgcacaccttcccagctgtcctacagtccta

SEQ ID NO: 80 - DNA, 494 bp, Homo sapiens
ggaattcggcttgatattcagctgactcagtctccatcctcactgtctgcatctgtaggagacagagtcaccatcacttgtcgggcgagtcagggcattagcatttatttagcctggtttcagcagagaccagggaaagcccctaagtccctgatctatgctgcatccagtttgcaaagtggggtcccatcaaagttcagcggcagtggatctgggacagatttcactctcaccatcagcagcctgcagcctgaagattttgcaacttattactgccaacaatataatagttacccattcactttcggccctgggaccaaagtggatatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaagccgaattc

SEQ ID NO: 81 - DNA, 1774 bp, Homo sapiens
atgtacttgggactgaactatgtattcatagtttttctcttaaatggtgtccagagtgaagtgaagcttgaggagtctggaggaggcttggtgcaacctggaggatccatgaaactctcctgtgttgcctctggattcactttcagtaactactggatgaactgggtccgccagtctccagagaaggggcttgagtgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtacggattacgatgcttactggggccaagggactctggtcactgtctctgcagagagtcagtccttcccaaatgtcttccccctcgtctcctgcgagagccccctgtctgataagaatctggtggccatgggctgcctggcccgggacttcctgcccagcaccatttccttcacctggaactaccagaacaacactgaagtcatccagggtatcagaaccttcccaacactgaggacagggggcaagtacctagccacctcgcaggtgttgctgtctcccaagagcatccttgaaggttcagatgaatacctggtatgcaaaatccactacggaggcaaaaacagagatctgcatgtgcccattccagctgtcgcagagatgaaccccaatgtaaatgtgttcgtcccaccacgggatggcttctctggccctgcaccacgcaagtctaaactcatctgcgaggccacgaacttcactccaaaaccgatcacagtatcctggctaaaggatgggaagctcgtggaatctggcttcaccacagatccggtgaccatcgagaacaaaggatccacaccccaaacctacaaggtcataagcacacttaccatctctgaaatcgactggctgaacctgaatgtgtacacctgccgtgtggatcacaggggtctcaccttcttgaagaacgtgtcctccacatgtgctgccagtccctccacagacatcctaaccttcaccatccccccctcctttgccgacatcttcctcagcaagtccgctaacctgacctgtctggtctcaaacctggcaacctatgaaaccctgaatatctcctgggcttctcaaagtggtgaaccactggaaaccaaaattaaaatcatggaaagccatcccaatggcaccttcagtgctaagggtgtggctagtgtttgtgtggaagactggaataacaggaaggaatttgtgtgtactgtgactcacagggatctgccttcaccacagaagaaattcatctcaaaacccaatgaggtgcacaaacatccacctgctgtgtacctgctgccaccagctcgtgagcaactgaacctgagggagtcagccacagtcacctgcctggtgaagggcttctctcctgcagacatcagtgtgcagtggcttcagagagggcaactcttgccccaagagaagtatgtgaccagtgccccgatgccagagcctggggccccaggcttctactttacccacagcatcctgactgtgacagaggaggaatggaactccggagagacctatacctgtgttgtaggccacgaggccctgccacacctggtgaccgagaggaccgtggacaagtccactggtaaacccacactgtacaatgtctccctgatcatgtctgacacaggcggcacctgctattgaccat

SEQ ID NO: 82 - protein, 81 aa, Homo sapiens
KVSCKASGYTFTSYDINWVRQATGQGLEWMGWMNPNSGNTGYAQKFQGRVTMTRNTSISTAYMELSSLRSEDTAVYYCARG

SEQ ID NO: 83 - protein, 92 aa, Homo sapiens
KVSCKASGYTFTSYDINWVRQATGQGLEWMGWMNPNSGNTGYAQKFQGRVTMTRNTSISTAYMELSSLRSEDTAVYYCAREEWLVRYYGMDV

SEQ ID NO: 84 - protein, 96 aa, Homo sapiens
KVSCKASGYTFTSYDINWVRQATGQGLEWMGWMNPNSGNTGYAQKFQGRVTMNRNTSISTAYMELSSLRSEDTAVYYCARGGHGGSYFYSYYGMDV

SEQ ID NO: 85 - protein, 78 aa, Homo sapiens
SLTCAVYGGFSGYYWSWIRQPPGKGLEWIGEINHSGSTNYNPSLKSRVTISVDTSKNQFSLKLSSVTAADTAVYYCAR

SEQ ID NO: 86 - protein, 92 aa, Homo sapiens
SLTCAVYGGFSGYYWSWIRQPPGKGLEWIGEINHSGSTNYNPSLKSRVTISVDTSKNQFSLKLSSVTAADTAVYYCARGAAEYYYYYYGMDV

SEQ ID NO: 87 - protein, 92 aa, Homo sapiens
SLTCAVYGGFSGYYWSWIRQPPGKGLEWIGEINHSGSTNYNPSLKSRVTISVDTSKNQFSLKLSSVTAADTAVYYCARGTTEYYYYYYGMDV

SEQ ID NO: 88 - protein, 79 aa, Homo sapiens
DRVTITCRASQGIRNDLGWYQQKPGKAPKRLIYAASSLQSGVPSRFSGSGSGTEFTLTISSLQPEDFATYYCLQHNSYP

SEQ ID NO: 89 - protein, 79 aa, Homo sapiens
DRVTITCRASQDIRDNLGWYQQKPGKAPKRLIYAASNLQSGVPSRFSGSGSGTEFTLTISSLQPEDFATYYCLQYKTYP

SEQ ID NO: 90 - protein, 79 aa, Homo sapiens
ERVTITCRASQGIRDELGWYQQKPGKAPKRLIYVASSLQSGVPSRFSGSGSGTEFTLTISSLQPEDFATYYCLQHNGYP

SEQ ID NO: 91 - protein, 85 aa, Homo sapiens
ERATINCKSSQSVLYSSNNKNYLAWYQQKPGQPPKLLIYWASTRESGVPDRFSGSGSGTDFTLTISSLQAEDVAVYYCQQYYSTP

SEQ ID NO: 92 - protein, 86 aa, Homo sapiens
ERATINCKSSQSVLYSFNNKNYLAWYQQKPGQPPKLLIYWASTRESGVPDRFSGSGSGTDFTLTISSLQAEDVAVYYCQQYYSTRT

SEQ ID NO: 93 - protein, 86 aa, Homo sapiens
ERATINCKSSQSVLYSFNNKNYLAWYQQKPGQPPKLLIYWASTRESGVPDRFGGSGSGTDFTLTISSLQAEDVAVYYCQQYYSTRT

SEQ ID NO: 94 - protein, 84 aa, Homo sapiens
EPASISCRSSQSLLHSNGYNYLDWYLQKPGQSPQLLIYLGSNRASGVPDRFSGSGSGTDFTLKISRVEAEDVGVYYCMQALQTP

SEQ ID NO: 95 - protein, 86 aa, Homo sapiens
EPASISCRSSQSLLHSNGYNYLDWYLQKPGQSPQLLIYLGSNRASGVPDRFSGSGSGTDFTLKISRVEAEDVGIYYCMQTRQTPRT

SEQ ID NO: 96 - protein, 86 aa, Homo sapiens
EPASISCRSSQSLLHSNGYNYLDWYLQKPGQSPQLLIYLGSNRASGVPDRFSGSGSGTDFTLKISRVEAEDVGIYYCMQSLQIPRL

SEQ ID NO: 97 - protein, 80 aa, Homo sapiens
DRVTITCRASQGISNYLAWFQQKPGKAPKSLIYAASSLQSGVPSKFSGSGSGTDFTLTISSLQPEDFATYYCQQYNSYPP

SEQ ID NO: 98 - protein, 83 aa, Homo sapiens
DRVTITCRASQDISIYLAWFQQRPGKAPKSLIYAASSLQSGVPSKFSGSGSGTDFTLTISSLQPEDFATYYCQQYNSYFTFGP

SEQ ID NO: 99 - protein, 83 aa, Homo sapiens
DRVTITCRASQGISIYLAWFQQRPGKAPKSLIYAASSLQSGVPSKFSGSGSGTDFTLTISSLQPEDFATYYCQQYNSYFTFGP

SEQ ID NO: 100 - DNA, 18 bp, Homo sapiens
  CDS (1)..(18)
gtgctagctttccaggag

SEQ ID NO: 101 - protein, 6 aa, Homo sapiens
VLAFQE

SEQ ID NO: 102 - DNA, 21 bp, Homo sapiens
  CDS (1)..(21)
cgcgtgctagctttccaggag

SEQ ID NO: 103 - protein, 7 aa, Homo sapiens
RVLAFQE

SEQ ID NO: 104 - DNA, 21 bp, Homo sapiens
  CDS (4)..(21)
cgtgtgctagctttccaggag

SEQ ID NO: 105 - protein, 6 aa, Homo sapiens
VLAFQE

(19) United States
(12) Patent Application Publication
(10) Pub. No.: US 2006/0104974 A1
(43) Pub. Date: May 18, 2006

(54) CD147 binding molecules as therapeutics

(76) Inventors: C. Geoffrey Davis, Burlingame, CA (US); Russell W. Blacher, Castro Valley, CA (US); Jose R. Corvalan, Foster City, CA (US); Alan R. Culwell, Carlsbad, CA (US); Larry L. Green, San Francisco, CA (US); Joanne Hales, Fremont, CA (US); Nancy Havrilla, Oakland, CA (US); Vladimir E. Ivanov, Fremont, CA (US); John A. Lipani, Livermore, CA (US); Qiang Liu, Foster City, CA (US); Richard F. Weber, San Francisco, CA (US); Xiao-Dong Yang, Palo Alto, CA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 09/784,950

(22) Filed: Feb. 15, 2001

Related U.S. Application Data
(63) Continuation of application No. PCT/US99/04583, filed on Mar. 3, 1999.
Application No. PCT/US99/04583 is a continuation-in-part of application No. 09/244,253, filed on Feb. 3, 1999, now abandoned.
Application No. 09/244,253 is a continuation-in-part of application No. 09/034,607, filed on Mar. 3, 1998, now abandoned.

(51) Int. Cl. A61K 39/395; C07K 16/28

(52) U.S. Cl. 424/144.1; 530/388.22

(57) ABSTRACT

In accordance with the present invention, we have discovered that the molecule CD147 as expressed on certain cells, such as T-cells, B-cells, and/or monocytes, can be utilized for the treatment of a variety of diseases. In particular, we have demonstrated that antibodies that bind to CD147 and that result in the killing of such cells, for example, through the binding of complement, is efficacious in the treatment of diseases. Diseases in which such treatment appears efficacious include, without limitation: graft versus host disease (GVHD), organ transplant rejection diseases (including, without limitation, renal transplant, ocular transplant, and others), cancers (including, without limitation, cancers of the blood (i.e., leukemias and lymphomas), pancreatic, and others), autoimmune diseases, inflammatory diseases, and others.

### BACKGROUND OF THE INVENTION

1. Summary of the Invention

In accordance with the present invention, we have discovered that the molecule CD147 as expressed on certain cells, such as T-cells, B-cells, and/or monocytes, can be utilized as a target for the treatment of a variety of diseases In particular, we have demonstrated that an antibody that binds to CD147 and that results in the killing of such cells, for example, through the binding of complement, is efficacious in the treatment of diseases Diseases in which such treatment appears efficacious include, without limitation. graft versus host disease (GVHD), organ transplant rejection diseases (including, without limitation, renal transplant, ocular transplant, and others), cancers (including, without limitation, cancers of the blood (i.e., leukemias and lymphomas) and pancreatic), autoimmune diseases (including, without limitation, lupus), inflammatory diseases (including, without limitation, arthritis), and others

2. Background of the Technology

In about 1982, a group from UCLA reported the generation of antibodies cytotoxic to human leukemia cells in mice through immunization with acute leukemia cells followed by formation of hybridomas and screening of the hybridomas in a microcytotoxicity assay in which toxicity of the antibody against the immunizing cells and normal lymphocytes was assayed. See U.S. Pat. Nos. 5,330,896 and 5,643,740, the disclosures of which are hereby incorporated by reference in their entirety One hybridoma was recovered that was cytotoxic to tumor cells but non-toxic to normal cells (except activated T-cells, activated B-cells, and monocytes were also killed) Such hybridoma was cloned and isolated and deposited with the ATCC as HB 8214. The monoclonal antibody expressed by this hybridoma was designated CBL1, and is a murine IgM. The group further demonstrated that the antibody was reactive with an antigenic determinant that appeared to be present in the cytoplasm of both activated and nonactivated cells. However, the antigenic determinant appeared to be present on the extracellular membrane of only certain circulating cells, including, activated T-cells, activated B-cells, and resting and activated monocytes, but not present extracellularly on other circulating nonactivated cells.

The group also endeavored to isolate the antigen responsible for the observations. The patents characterize the antigenic determinant recognized by the CBL-1 antibody as being a molecule that:
(i) is present on the cell membrane and within the cytoplasm of tumor cells and activated lymphocytes;
(ii) is present in the cytoplasm of unstimulated normal peripheral blood lymphocytes but when these cells are stimulated by antigens or by mitogens, said antigen appears also on the cell membrane;
(iii) is present on lymphocytes activated in vitro by mitogens;
(iv) is capable of binding to CBLI monoclonal antibody which is produced by the hybridoma cell line having the ATCC number HB8214;
(v) functions as an autocrine growth factor produced by tumor cells and activated lymphocytes;
(vi) binds to the surface membrane of tumor cells and stimulates the growth of these cells and cells of the lymphoid series;
(vii) is present in the medium from growing cancer cells and in the serum of patients with cancer and diseases in which activated lymphocytes are present; and
(viii) has a molecular weight of approximately 15,000 daltons.

No improved identification of the antigen to which the CBL1 antibody binds has been accomplished with respect to the UCLA group's papers and patents. Nevertheless, the CBL1 antibody has been effective in patients in the treatment of a variety of diseases including: graft versus host disease (GVHD) and kidney transplant rejection. See e.g., Heslop et al. The Lancet 346:805-806 (1995) (GVHD); Benamin Clinical Trial Monitor Abstract No. 13385 (1995); Takahashi et al. The Lancet 2:1155-1158 (1983) (kidney allograft rejection); Takahashi Transplantation Proceedings 17:10-12 (1985) (kidney allograft rejection); Oei et al. Transplantation Proceedings 17:13-16 (1985) (kidney allograft rejection). In connection with such studies, there has been no evidence of safety concerns or cross-reactivity. The following papers relate to additional characterization of the CBL1 antibody: Billing et al. Hybridoma 1:303-311 (1982); Billing et al. Clin. Exp. Immunol. 49:142-148 (1982); Chatterjee et al. Hybridoma 1:369-377 (1982); Billing R. and Chatterjee S. Transplantation Proceedings 15:649-650 (1983); Kinukawa T. and Terasaki P. I. Transplantation Proceedings 1:993-998 (1985); Billing in Monoclonal Antibodies: Diagnostic and Therapeutic Use in Tumor and Transplantation Ch. 9, 85-90 (Chatterjee ed., PSG Publ. Co., Inc. (1985)); Billing et al. in Monoclonal Antibodies: Diagnostic and Therapeutic Use in Tumor and Transplantation Ch. 2, 11-19 (Chatterjee ed., PSG Publ. Co., Inc. (1985)).

Human Graft Versus Host Disease (GVHD) was first described by Mathe et al. in 1960 (Mathe et al. “Nouveaux essais de greffe de moelle osseuse homologue apres irradiation totale chez des enfants atteints de leucemie aigue en remission. Le probleme du syndrome secondaire chez l'homme” Rev Fr Etud Clin Biol 15:115-161 (1960)). Essentially GVHD is the clinical manifestation of an immunological reaction between donor cells and host tissue. The clinical syndrome consists of skin rash, gastro-intestinal symptoms, and hepatic dysfunction seen usually within two weeks of allogeneic bone marrow transplant. The immunopathogenesis requires recognition of host antigens by immunocompetent donor cells; immunosuppressed host (recipient); and alloantigenic differences to exist between donor and recipient. The immunocompetent donor cells are mature T-cells (Ferrara J L and Deeg H J “Graft versus Host Disease” NEJM 324:667 (1991) and the clinical severity of the disease correlates with the number of T-cells transferred to the patient (Ferrara J L and Deeg H J “Graft versus Host Disease” NEJM 324:667 (1991).

The clinical features of acute GvHD include dermatitis, jaundice and gastro-intestinal involvement. These symptoms may occur alone or in any combination and can range from mild to life-threatening. Skin involvement is the most common manifestation. The most severe manifestation of skin involvement includes bullous lesions similar to third degree burns. Jaundice is brought about from an elevated bilirubin with and without alteration of other liver enzymes. Gastro-intestinal involvement includes watery diarrhea. This diarrhea can be voluminous and bloody, causing life-threatening fluid and electrolyte losses as well as a portal of entry for infections. Other patients may experience severe ileus. Upper GI involvement is less common. This presents as anorexia, dyspepsia, food intolerance and nausea/vomiting. Most patients with GI involvement require total parenteral nutrition (TPN) support.

Strategies for prevention and possibly treatment should be and sometimes are, directed towards removal of T-cells from the donor marrow or toward blocking their activation. However, the T-depleted marrow results in a higher rate of graft failure that is usually fatal. An additional concern associated with T-depleted marrow is the increased relapse rate in marrow recipients with a primary diagnosis of leukemia. A graft versus leukemia effect, mediated by donor T-cells, also mitigates against using a T-depleted marrow in allogeneic bone marrow transplantation.

Clinically significant acute GVHD (Grades II-IV) occurs in up to 50% of patients who receive a marrow from a HLA genotypically identical sibling. If unrelated matched donors are used, the incident increases to 80% in some studies. The greater the HLA incompatibility, the greater the incidence and severity of GVHD.

The primary treatment for acute GvHD is prevention. Prevention regimens include the use of immunosuppression therapy and T-cell depletion of the donor cells. “Standard” first-line therapy consists of glucocorticoids. Approximately 20-25% of patients achieve a complete response and patients who do not respond have a poor outcome. Those patients who continue to require treatment with steroids are susceptible to all of the untoward effects of steroid use. These untoward effects include increased susceptibility to infections, GI bleed, altered metabolic states, hypertension, etc.

Glucocorticoids, cyclosporine, methotrexate, cyclophosphamide have all been used in prevention as well as treatment of GVHD. Anti-thymocyte globulin (ATG) has been used for many years. All of these agents are potentially quite toxic. Monoclonal antibodies such as anti-Interleukin-2 and immunotoxins like anti-CD5-ricin have been used and found to be of limited success. A humanized anti-TAC was used for prophylaxis of GVHD but failed in the treatment protocols.

Because of the indication that CBL1 was effective in treating GVHD, we undertook additional investigations of the CBL1 antibody. In connection with such additional work, we have now demonstrated that the CBL1 antibody, in fact, appears to bind to and be efficacious with respect to the CD147 antigen as expressed on certain cells, such as T-cells, B-cells, and/or monocytes through the process of complement dependent cytotoxicity (killing).

CD147 is a member of the immunoglobulin (Ig) superfamily that is expressed on a large number of different cells in a variety of tissues. It was originally named human Basigin (for basic immunogloblin superfamily) and was first cloned in about 1991. (Miyauchi et al. J Biochem (Tokyo) 110:770-774 (1991); Kanekura et al. Cell Struct Funct 16:23-30 (1991); Miyauchi et al. J Biochem (Tokyo) 110:770-774 (1991)). The molecule is composed of approximately 269 amino acids (Miyauchi et al. J Biochem (Tokyo) 110:770-774 (1991)) and is a glycoprotein with about 40% of its molecular weight made up of carbohydrate, having a predicted deglycosylated molecular weight of approximately 27 KD and a fully glycosylated molecular weight of between 43-66 KD (Kanekura et al. Cell Struct Funct 16:23-30 (1991)). The Basigin gene was mapped to Chromosome 19p13.3 (Kaname et al. Cytogenet Cell Genet 64:195-197 (1993)).

The molecule has been identified to possess homology with, or identity to, a number of other molecules, including:

Mouse Basigin (Miyauchi et al. J Biochem (Tokyo) 107:316-323 (1990); Joseph et al. Adv Exp Med Biol 342:389-391(1993); Kaname et al. J Biochem (Tokyo) 118:717-724 (1995));

Rabbit Basigin (Schuster et al. Biochim Biophys Acta 1311:13-19 (1996));

Mouse gp42 (Altruda et al. Gene 85:445-451 (1989); Imboden et al. J Immunol 143:3100-3103 (1989); Cheng et al. Biochim Biophys Acta 1217:307-311 (1994));

Chicken HT7 or 5A11 (Albrecht et al. Brain Res 535:49-61 (1990); Seulberger et al. EMBO J 9:2151-2158 (1990); Miyauchi et al. J Biochem (Tokyo) 110:770-774 (1991); Janzer et al. Adv Exp Med Biol 331:217-221 (1993); Lobrinus et al. Brain Res Dev Brain Res 70:207-211 (1992); Seulberger et al. Neurosci Lett 140:93-97 (1992); Fadool J M & Linser P J J Neurochem 60:1354-136 (1993); Fadool J M & Linser P J Dev Dyn 196:252-262 (1993); Unger et al. Adv Exp Med Biol 331:211-215 (1993); Rizzolo L J & Zhou S J Cell Sci 108:3623-3633 (1995); Ikeda et al. Neurosci Lett 209:149-152 (1996); Fadool J M & Linser P J Biochem Biophys Res Commun 229:280-286 (1996));

Neurothelin (Schlosshauer B & Herzog K H J Cell Biol 110:1261-1274 (1990); Schlosshauer B Development 113:129-140 (1991); Schlosshauer B BioEssays 15:341-346 (1993); Schlosshauer et al. Eur J Cell Biol 68:159-166 (1995));

M6 leukocyte activation antigen (Felzmann et al. J Clin Immunol 11:205-212 (1991); Gadd et al. Rheumatol Int 12:153-157 (1992); Kasinrerk et al. J Immunol 149:847-854 (1992));

OX-47 (Fossum et al. Eur J Immunol 21:671-679 (1991); Fossum et al. Eur J Immunol 21:671-679 (1991); Casselia et al. J Anat 189:407-415 (1996));

Mo3 (Mizukami et al. J Immunol 147:1331-1337 (1991));

CE9 (Petruszak et al. J Cell Biol 114:917-927 (1991); Scott L J & Hubbard A L J Biol Chem 267:6099-6106 (1992); Nehme et al. J Cell Biol 120:687-694 (1993); Cesario M M & Bartles J R J Cell Sci 107:561-570 (1994); Cesario et al. Dev Biol 169:473-486 (1995); Nehme et al. Biochem J 310:693-698 (1995));

EMMPRIN (Biswas et al. Cancer Res 55:434 (1995); DeCastro et al. J Invest Dermatol 106:1260-1265 (1996));

RET-PE2 (Finnemann et al. Invest Ophthalmol Vis Sci 38:2366-2374 (1997));

Ok^{a} Blood Group Antigen (Spring et al. Eur J Immunol 27:891-897 (1997)); and

1W5 (Seulberger et al. EMBO J 9:2151-2158 (1990)).

Indeed, Seulberger et al. Neurosci Lett 140:93-97 (1992) demonstrated that HT7, Neurothelin, Basigin, gp42 and OX-47 were each names for one molecule which is a developmentally regulated immunoglobulin-like surface glycoprotein which is present on blood-brain barrier endothelium, epithelial tissue barriers, and neurons. Further, Kasinrerk et al. J Immunol 149:847-854 (1992) demonstrated that the human leukocyte activation antigen M6 is a member of the Ig superfamily and is the species homologue of rat OX47, mouse Basigin, and chicken HT7 antigens. EMMPRIN was demonstrated to be identical to the M6 antigen and human Basigin (Biswas et al. Cancer Res 55:434 (1995)). See also Guo et al. “Characterization of the gene for human EMMPRIN, a tumor cell surface inducer of matrix metalloproteinases” Gene 220:99-108 (1998) conducted additional characterization of the gene for human EMMPRIN;

Through its homology with the related molecules, CD147 has been shown or postulated to have a role in a number of physiological processes, diseases, and/or conditions. For example, an early role postulated for the molecule was activity in the blood-brain barrier. Such relationship was first demonstrated with respect to the chick HT7 antigen (Risau et al. EMBO J 5:3179-3183 (1986); Albrecht et al. Brain Res 535:49-61 (1990); Seulberger et al. EMBO J 9:2151-2158 (1990); Janzer et al. Adv Exp Med Biol 331:217-221 (1993); Lobrinus et al. Brain Res Dev 70:207-211 (1992); Unger et al. Adv Exp Med Biol 331:211-215 (1993)). A similar relationship was observed in connection with Neurothelin (Schlosshauer B & Herzog K H J Cell Biol 110:1261-1274 (1990); Schlosshauer B Development 113:129-140 (1991); Schlosshauer B BioEssays 15:341-346 (1993); Schlosshauer et al. Eur J Cell Biol 68:159-166 (1995)). The molecule has also been postulated to be involved in development and activation of various cells, for example: lymphocyte activated killer (LAK) cell activation (Imboden et al. J Immunol 143:3100-3103 (1989)), T-cell activation (Paterson et al. Mol Immunol 24:1281-1290 (1987); Kirsch et al. Tissue Antigens 50:147-152 (1997)), leukocyte activation (Fossum et al. Eur J Immunol 21:671-679 (1991); Fossum et al. Eur J Immunol 21:671-679 (1991)), and mononuclear phagocyte activation (Mizukami et al. J Immunol 147:1331-1337 (1991)). Other regulatory, signaling, and recognition functions have also been postulated, for instance: MHC function (Miyauchi et al. J Biochem (Tokyo) 107:316-323 (1990)), signal transduction and membrane transport (Kasinrerk et al. J Immunol 149:847-854 (1992); Berditchevski et al. J Biol Chem 272:29174-29180 (1997)), cellular recognition (Fadool J M & Linser P J Dev Dyn 196:252-262 (1993); Kaname et al. Cytogenet Cell Genet 64:195-197 (1993)), cellular adhesion (Miyauchi et al. J Biochem (Tokyo) 110:770-774 (1991); Seulberger et al. Neurosci Lett 140:93-97 (1992); Joseph et al. Adv Exp Med Biol 342:389-391 (1993); Sudou et al. J Biochem (Tokyo) 117:271-275 (1995)), intercellular stimulation and matrix metalloproteinase synthesis (Biswas et al. Cancer Res 55:434 (1995)), tissue remodeling (Guo et al. J Biol Chem 272:24-27 (1997)), metabolism, and sperm development and maturation (Petruszak et al. J Cell Biol 114:917-927 (1991); Nehme et al. J Cell Biol 120:687-694 (1993); Cesario M M & Bartles J R J Cell Sci 107:561-570 (1994); Cesario et al. Dev Biol 169:473-486 (1995)). CD147 also appears to have a role in retinal development and disease, see Marmorstein et al. “Morphogenesis of the retinal pigment epithelium: toward understanding retinal degenerative diseases” Ann N Y Acad Sci 857:1-12 (1998) (suggested that N-CAM and EMMPRIN are potentially important molecules in other RPE functions necessary for photoreceptor survival). See also Marmorstein et al. “Apical polarity of N-CAM and EMMPRIN in retinal pigment epithelium resulting from suppression of basolateral signal recognition” J Cell Biol 142:697-710 (1998).

The molecule has also been investigated for a potential association in both rheumatoid and reactive arthritis (Felzmann et al. J Clin Immunol 11:205-212 (1991); Gadd et al. Rheumatol Int 12:153-157 (1992)) and renal disease (Schuster et al. Biochim Biophys Acta 1311:13-19 (1996)). Moreover, certain clear associations between the molecule and cancer have also been indicated (Biswas Biochem Biophys Res Commun 109:1026 (1982); Miyauchi et al. J Biochem (Tokyo) 110:770-774 (1991); Biswas et al. Cancer Res 55:434 (1995); Guo et al. J Biol Chem 272:24-27 (1997); Guo et al. J Biol Chem 272:24-27 (1997)). See also Lim et al. “Tumor-derived EMMPRIN (extracellular matrix metalloproteinase inducer) stimulates collagenase transcription through MAPK p38” FEBS Lett 441:88-92 (1998); van den Oord et al. “Expression of gelatinase B and the extracellular matrix metalloproteinase inducer EMMPRIN in benign and malignant pigment cell lesions of the skin” Am J Pathol 151:665-70 (1997); Polette et al. “Tumor collagenase stimulatory factor (TCSF) expression and localization in human lung and breast cancers” J Histochem Cytochem 45:703-9 (1997).

A mouse model in which the Basigin gene was knocked-out has been examined (Igakura et al. Biochem Biophys Res Commun 224:33-36 (1996)). The work indicated that the molecule was not necessarily active in the blood-brain barrier. However, the work indicated that there was enhanced interaction in connection with lymphocyte activation as well as an abnormal response to irritating odors. Later work indicated certain abnormalities in sensory and memory functions in such model Naruhashi et al. Biochem Biophys Res Commun 236:733-737 (1997)).

In connection with the expression of CD147, see Woodhead et al. “From sentinel to messenger: an extended phenotypic analysis of the monocyte to dendritic cell transition” Immunology 94:552-9 (1998) demonstrated that CD147 was expressed on dendritic cells, Ghannadan et al. “Phenotypic characterization of human skin mast cells by combined staining with toluidine blue and CD antibodies” J Invest Dermatol 111:689-95 (1998) demonstrated that clustered CD antigens (including CD147) were detectable foreskin mast cells, Mutin et al. “Immunologic phenotype of cultured endothelial cells: quantitative analysis of cell surface molecules” Tissue Antigens 50:449-58 (1997) discussed quantitative analysis of cell surface molecules on cultured endothelial cells (HUVEC).

In view of the foregoing, CD147 has been implicated as a potentially useful target for the treatment of diseases. However, at the same time, CD147 is expressed in and on many cells that are widely distributed amongst many tissues. For example, the Ok^{a} blood group antigen is expressed on virtually all cells (Williams et al. Immunogenetics 27:322-329 (1988)). OX-47 has been disclosed to be on most immature cells, endothelial cells, and cells with excitable membranes (Fossum et al. Eur J Immunol 21:671-679 (1991)). Similarly, Basigin was demonstrated to be expressed not only in endothelial cells but was also found in a variety of tissues, including, the spleen, small intestine, kidney, and liver in relatively high levels and in small quantities in the testes (Kanekura et al. Cell Struct Funct 16:23-30 (1991)). CE9 was disclosed to be widely expressed on rat hepatocytes (Scott L J & Hubbard A L J Biol Chem 267:6099-6106 (1992)). Seulberger et al. Neurosci Lett 140:93-97 (1992) demonstrated that the HT7 molecule (which is identical to Neurothelin, Basigin, gp42, and OX-47) was expressed on the blood-brain barrier, chloroid plexus (blood-CNS fluid barrier), retinal epithelium (blood-eye barrier), neurons, kidney tubules, some endothelium, epithelium, and epithelial tissue barriers. The CE9 antigen (which was demonstrated to possess identity to the OX-47 antigen) is expressed, to some extent, in virtually all rat tissues (Nehme et al. Biochem J 310:693-698 (1995)). Because of the broad tissue distribution, there would be a number of concerns related to the safety of any therapy that inhibited or killed cells expressing it.

There is some evidence that there may be different forms of CD147, stemming from, for example, differential glycosylation or alternative splicing of the molecule (Kanekura et al. Cell Struct Funct 16:23-30 (1991) (Basigin); Schlosshauer B Development 113:129-140 (1991) (Neurothelin); Fadool J M & Linser P J J Neurochem 60:1354-136 (1993) (5A11/HT7); Nehme et al. J Cell Biol 120:687-694 (1993) (CE9); DeCastro et al. J Invest Dermatol 106:1260-1265 (1996) (EMMPRIN); Spring et al. Eur J Immunol 27:891-897 (1997) (Ok^{a})).

### BRIEF DESCRIPTION OF THE DRAWING FIGURES

FIG. 1 is a 12% SDS-PAGE/Western Blot showing the binding of particular antibodies to CEM cell membrane extracts lysates. Lane A: rabbit-anti-mouse-hn-RNP-K protein antibody; Lane B: ABX-CBL antibody; Lane C: 2.6.1 antibody (also referred to herein as cem2.6 and ABX-Rb2); Lane D: anti-CD147 antibody (Pharmingen); and Lane E: anti-CD147 antibody (RDI). Sample: 5 microliters CEM Cell Extract.

FIGS. 2A-2B is an analysis of the components obtained from the CBL1 antibody produced by the hybridoma cell line having ATCC Deposit No. BB 8214. The data demonstrate that the CBL1 IgM antibody produced by the HB 8214 hybridoma is the active component that inhibits MLR in the presence of complement.

FIG. 3 is a graph comparing the inhibition of MLR using antibodies from various CBL1 subclones in comparison to CBL1.

FIG. 4 is a graph comparing MLR inhibition utilizing ABX-CBL in the presence of rabbit and human complement.

FIG. 5 is a graph comparing the activity of the ABX-CBL antibody and the 2.6.1 antibody (also referred to as cem 2.6) in inhibiting the MLR assay. The data demonstrate that the 2.6.1 antibody is not an effective inhibitor.

FIGS. 6A-6B: FACS analyses of activated lymphocytes demonstrating co-expression of CD147 and CD25.

FIGS. 7A-7D: FACS analyses of PBMC demonstrating the selective upregulation of CD25 upon stimulation, and the specific depletion of the same cells after treatment with ABX-CBL and complement. FIG. 7A: untreated PBMC. FIGS. 7B and 7D: PBMC stimulated with ConA. FIG. 7C: PBMC stimulated with ConA, then treated with ABX-CBL plus complement.

FIGS. 8A-8D compare FACS analyses of PBMC demonstrating the selective upregulation of CD25 upon stimulation, and the specific depletion of the same cells after treatment with ABX-CBL and complement. FIG. 8A: PBMC+ConA; FIG. 8B: CBL-1 only/Medium; FIG. 8C: Complement only/Medium; FIG. 8D: CBL-1+complement/Medium. M1: CD25 high (depleted); M2: CD25 low (undepleted); M3: CD25 null (undepleted).

FIGS. 9A-9D show another series of FACS analyses of PBMC demonstrating the selective upregulation of CD25 and CD147 upon stimulation.

FIGS. 10A-10F show a comparison of activated T-cells (FIGS. 10A-10B), activated monocytes, (FIGS. 10C-10-D) and activated B-cells (FIGS. 10E-10F) before and after treatment with ABX-CBL and complement and demonstrating the specific depletion of the same cells upon treatment with ABX-CBL and complement.

FIGS. 11A-11F shows a similar comparison of subpopulations of activated T-cells (FIGS. 11A-11B), activated B-cells (FIGS. 11C-11D), and activated monocytes (FIGS. 11E-11F) before and after treatment with ABX-CBL and complement. The data demonstrate the specific depletion of the same cells upon treatment with ABX-CBL and complement.

FIG. 12 illustrates that the mode of action of ABX-CBL is by depleting leukocyte subpopulations. The table compares cell type, surface markers, and Complement-Dependent Cytotoxicity (CDC) depletion of leukocyte subpopulations.

FIG. 13 is a table comparing cell, cell type, CD147 expression, and CDC after treatment of the cells with ABX-CBL and complement. The data demonstrate that not all cells that express CD147 are killed upon such treatment.

FIG. 14 is a table summarizing the expression of CDC resistant molecules on CBL-1⁺ cells. The chart compares cell, cell type, CD147 expression, CDC after treatment of the cells with ABX-CBL and complement, and expression of the complement inhibitory molecules CD55 and CD59. The data demonstrate that of these cells, only cells that do not express both CD55 and CD59 are killed upon such treatment.

FIGS. 15A-15C present FACS analyses showing the expression of CD147 on the human endothelial cell line ECV-304.

FIGS. 16A-16C present FACS analyses showing the expression of CD147 on the human endothelial cell line HUVEC-C.

FIG. 17 is a graph showing the effects of ABX-CBL and complement on the human endothelial cell line ECV-304 in comparison to the effects of the same on CEM cells.

FIG. 18 is a graph showing the effect of ABX-CBL on human endothelial cell line HUVEC-C in comparison to the effects of the same on CEM cells.

FIGS. 19A-19C present FACS analyses showing the expression of the complement inhibitory molecules CD46, CD55, and CD59 on the human endothelial cell line ECV-304.

FIGS. 20A-20C present FACS analyses showing the expression of the complement inhibitory molecules CD46, CD55, and CD59 on the human endothelial cell line HUVEC-C.

FIG. 21 is a schematic diagram of the vector utilized for cloning and expression of CD147 cDNA in COS cells.

FIG. 22 is a schematic diagram of the pBK-CMN phagemid vector utilized for cloning and expression of CD147 cDNA in COS and E. coli cells.

FIG. 23 is a SDS-PAGE/Western Blot of CD147 expressed in COS cells (FIG. 23A) and E. coli (FIG. 23B). FIGS. 23A-23B: Antibodies: Pharmingen (panel A), 2.6.1 (panel B), and ABX-CBL (panel C). FIG. 23A: 5 μL CEM cell membrane extract (Lane 1); 7.5 μL control vector transfected COS cell extract (Lane 2); 7.5 μL CD147 transfected COS cell extract (Lane 3). FIG. 23B: Clone 1: CD147-Transfected, uninduced (Lane 1); Clone 1: CD147-Transfected, induced (Lane 2); Clone 5: Control Vector Transfected, uninduced (Lane 3); Clone 5: Control Vector Transfected, induced (Lane 4).

FIGS. 24-33 are heavy chain and kappa chain cDNA and protein sequences of or for the antibodies: CEM 10.1 C3 (FIG. 24), CEM 10.1 G10 (FIG. 25), CEM 10.12 F3 (FIG. 26), CEM 10.12 G5 (FIG. 27), CEM 13.12 (FIG. 28), CEM 13.5 (FIG. 29), 2.4.4 (FIG. 30), 2.1.1 (FIG. 31), 2.3.2 (FIG. 32), and 2.6.1 (FIG. 33).

FIGS. 34-43 are heavy chain and kappa chain protein sequences of or for the antibodies: CEM 10.1 C3 (FIG. 34), CEM 10.1 G10 (FIG. 35), CEM 10.12 F3 (FIG. 36), CEM 10.12 G5 (FIG. 37), CEM 13.12 (FIG. 38), CEM 13.5 (FIG. 39), 2.4.4 (FIG. 40), 2.1.1 (FIG. 41), 2.3.2 (FIG. 42), and 2.6.1 (FIG. 43) showing CDR positions.

FIGS. 44A-44B show the amino acid sequences and structure of human heavy chains derived from CBL-1 specific hybridomas showing alignment against the germline V-segment genes.

FIGS. 45A-45C and FIG. 46 show amino acid sequences and structure of human kappa chains derived from CBL-1 specific hybridomas, showing alignment against the germline V-segment genes.

FIG. 47 is a restriction map of the vector pWBFNP MCS that was utilized for the construction and cloning of certain constructs in accordance with the invention.

FIG. 48 is a schematic restriction map of the vector pIK6.1+Puro that was utilized for the construction and cloning of certain constructs in accordance with the invention.

FIG. 49 shows a comparison of the activity of the ABX-CBL antibody and the 2.6.1 multimeric IgM antibody (also known as ABX-Rb2) in inhibiting the MLR assay, demonstrating that the 2.6.1 multimeric IgM antibody is effective in inhibition of MLR. C: Rabbit complement.

FIGS. 50A-50F provide additional detail of the cloning strategy utilized in connection with the generation of CD147-IgG2 and gp42-IgG2 fusion proteins for use in connection with the generation of surrogate antibodies for use in animal models.

### SUMMARY OF THE INVENTION

In accordance with a first aspect of the present invention, there is provided an isolated monoclonal antibody having an isotype that fixes complement and a variable region that binds to the epitope on CD147 bound by the IgM monoclonal antibody ABX-CBL, with the proviso that the antibody is not CBL1. In a preferred embodiment, the antibody in the presence of complement acts to selectively kill cells selected from the group consisting of activated T-cells, activated B-cells, and monocytes but is substantially non-toxic to resting T-cells and resting B-cells. In another preferred embodiment, the antibody is a human antibody. In another preferred embodiment, the antibody has an isotype is selected from the group consisting of murine IgM, murine IgG2a, murine IgG2b, murine IgG3, human IgM, human IgG1, and human IgG3.

In accordance with a second aspect of the present invention, there is provided an isolated monoclonal antibody having an isotype that fixes complement and a variable region that binds to CD147 on populations of activated T-cells, activated B-cells, and resting or activated monocytes, that, in the presence of complement, selectively depletes such populations through complement mediated killing while being substantially nontoxic to other cells, with the proviso that the antibody is not CBL1. In a preferred embodiment, the antibody is a human antibody. In another preferred embodiment, the antibody has an isotype selected from the group consisting of murine IgM, murine IgG2a, murine IgG2b, murine IgG3, human IgM, human IgG1, and human IgG3.

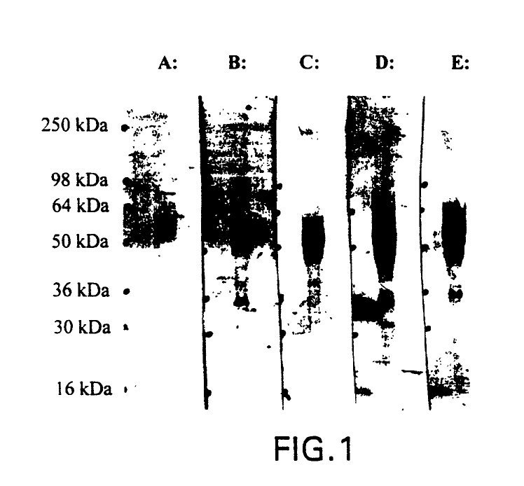

In accordance with a third aspect of the present invention, there is provided an isolated monoclonal antibody having the following characteristics: binds to CD147; shows a binding against CEM cell lysates on Western blot similar to that provided in FIG. 1; an isotype selected from the group consisting of murine IgM, murine IgG2a, murine IgG2b, murine IgG3, human IgM, human IgG1, and human IgG3, competes with ABX-CBL for binding to CD147; cross reacts with hn-RNP-k protein; binds to a consensus sequence on CD147 comprising RVRS; selectively kills activated T-cells, activated B-cells, and monocytes in a MLR assay only in the presence of complement; and is substantially non-toxic to cells expressing CD55 and CD59, with and without the presence of complement, with the proviso that the antibody is not CBL1.

In accordance with a fourth aspect of the present invention, there is provided a method to select an anti-CD147 antibodies for the treatment of disease, comprising: generating antibodies that bind to CD147 and that are capable of binding complement; assaying the antibodies for one or more of the following properties: competition with ABX-CBL for binding to CD147; capability to selectively kill activated T-cells, activated B-cells, and monocytes in a MLR assay only in the presence of complement; and being substantially non-toxic to cells expressing CD55 and CD59, with and without the presence of complement, with the proviso that the antibody is not CBL1. In a preferred embodiment, the method comprises assaying the antibodies for binding to CEM cell lysates on Western blot in a manner similar to that provided in FIG. 1. In another preferred embodiment, the method comprises assaying the antibodies for binding to a consensus sequence in a peptide of RXRS. In another preferred embodiment, the method comprises assaying the antibodies for cross reaction with hn-RNP-k protein. In another preferred embodiment, the method comprises assaying the antibodies for binding to a form of CD147 expressed by COS cells and E. coli cells.

In accordance with a fifth aspect of the present invention, there is provided a method for preventing or lessening the severity of disease, comprising providing to a subject in need of such treatment an antibody that has an isotype that fixes complement and a variable region that binds to CD147 on populations of activated T-cells, activated B-cells, and resting or activated monocytes, that, in the presence of complement, selectively depletes such populations through complement mediated killing while being substantially nontoxic to other cells, with the proviso that the antibody is not CBL1. In a preferred embodiment, the antibody is a human antibody. In another preferred embodiment, the antibody has an isotype is selected from the group consisting of murine IgM, murine IgG2a, murine IgG2b, murine IgG3, human IgM, human IgG1, and human IgG3.

In accordance with a sixth aspect of the present invention, there is provided a method to prevent or lessen the severity of GVHD, comprising providing to a subject in need of such treatment an antibody that has an isotype that fixes complement and a variable region that binds to CD147 on populations of activated T-cells, activated B-cells, and resting or activated monocytes, that, in the presence of complement, selectively depletes such populations through complement mediated killing while being substantially nontoxic to other cells, with the proviso that the antibody is not CBL1. In a preferred embodiment, the antibody is a human antibody. In another preferred embodiment, the antibody has an isotype is selected from the group consisting of murine IgM, murine IgG2a, murine IgG2b, murine IgG3, human IgM, human IgG1, and human IgG3.

In accordance with a seventh aspect of the present invention, there is provided a monoclonal antibody that binds to an epitope on CD147 comprising the consensus sequence RVRSH, wherein the antibody is not CBL1. In a preferred embodiment, the antibody is a human antibody.

In accordance with an eighth aspect of the present invention, there is provided an isolated peptide comprising the sequence selected from the group consisting of RXRS, RXRSH, RVRS, and RVRSH. In a preferred embodiment, the peptide is used for the generation of antibodies.

In accordance with a ninth aspect of the present invention, there is provided a human monoclonal antibody that binds to CD147.

### DETAILED DESCRIPTION OF THE PREFERRED EMBODIMENTS

Discussion of the Present Invention

The pharmaceutical agent ABX-CBL was derived from the hybridoma cell line expressing the CBL1 antibody. CBL1 is a murine IgM, anti-human lymphoblastoid monoclonal antibody that was raised in Balb/c mice immunized with the T cell acute lymphoblastic leukemia cell line (T-ALL) CEM (Billing et al. “Monoclonal and heteroantibody reacting with different common antigens common to human blast cells and monocytes” Hybridoma 1:303-311 (1982)). Following fusion of the splenocytes and selection in HAT medium, supernatants from hybridoma-containing wells were screened by microcytoxicity assay for reactivity with CEM cells. Hybridomas that tested positive in this assay were further screened for their ability to discriminate between resting lymphocytes and blast cells. CBL1 was selected for further study because it showed selectivity for blast cells (Billing et al. “Monoclonal and heteroantibody reacting with different common antigens common to human blast cells and monocytes” Hybridoma 1:303-311 (1982)). The CBL1 antibody was deposited with the ATCC as HB 8214.

The assignee of the present application, Abgenix, Inc., Fremont, Calif., acquired CBL1 in 1997 and determined that the hybridoma line deposited with the ATCC as HB 8214 was not entirely pure. Rather, it was actually a mix of two distinct hybridoma lines, one producing an IgG and one producing an IgM. Following subcloning, a pure IgM producer as well as a pure IgG producer were derived. Through a series of in vitro experiments described herein, it was demonstrated that the IgM antibody mediated the activities previously attributed to the CBL1 hybridoma. Only the IgM is biologically active in inhibition of complement mediated lysis of cells in a mixed lymphocyte reaction assay (MLR). The mechanism of inhibition is via antibody mediated complement-dependent cytotoxicity (CDC) because the inhibition is specific and complement-dependent, as discussed herein. Therefore, in connection with our work described herein, using conventional techniques, we subcloned the line to produce a cell line producing solely the IgM. Further, the HB 8214 cell line expressing the CBL1 antibody possessed a second kappa light chain (MOPC-21) which appears to have been derived from the myeloma fuision partner, a P3 myeloma cell line, that was used to prepared the original hybridoma cell line. Our subcloned hybridoma cell line possesses and expresses both light chains and the ABX-CBL antibody appears to contain both light chains. IgM antibodies generally possess a pentameric structure, where five heavy and light chain dimers are associated. With the two light chains in the ABX-CBL antibody, we expect that the IgM pentameric structure of the ABX-CBL antibody contains both light chains in various ratios of light chains to form pentamers with homodimeric, heterodimeric, and homo- and heterodimeric combinations.

In order to manufacture the ABX-CBL antibody for use in preclinical and clinical development, we utilized hollow fiber cell culture technology through contract manufacturing with Goodwin Biotechnology, Plantation, Fla. The growth medium is a serum free formulation HYBRIDOMA-SFM supplied by Gibco Life Technologies.

The stability of the Master Cell Bank (MCB) of ABX-CBL was determined by single cell subcloning. Cells were subdloned showing >95% stability for the single cell colony producers. The ABX-CBL MCB also showed stable antibody production for more than 130 generations in culture. The manufacturing process in hollow fiber bioreactors is an approximately 40 day growth process that is equivalent to approximately 130 generations.

Primary purification of the monoclonal antibody from the cell culture supernatant is performed using Protein A affinity chromatography. Incubation at low pH following elution is performed as a viral inactivation step. The material is further purified by anion exchange chromatography. This provides for residual protein A and DNA removal. The final step in the purification process is a filtration of the material to provide additional viral removal.

The formulated bulk drug substance is stored at 2-8° C. prior to vialing. Using aseptic techniques, the antibody is filled in liquid form from the bulk containers into 5 mL glass vials. The vials are stored and shipped at 2-8° C. ABX-CBL is a murine IgM, anti-human lymphoblastoid monoclonal antibody raised to a T-ALL (Acute Lymphoblastic Leukemia) cell line (CEM). ABX-CBL is formulated in 20 mM sodium citrate and 120 mM sodium chloride at a pH of 6.0.

As used herein, the term “ABX-CBL” is used to refer to the purified and reactive IgM antibody derived from the original cell line deposited with the ATCC as HB 8214. The sequence of the ABX-CBL heavy and light chains are discussed above and presented as SEQ ID NO.: 18 and SEQ ID NO.: 19, respectively.

We have now demonstrated that the active agent of the CBL1 antibody and ABX-CBL binds to the CD147 antigen as expressed on certain cells, such as T-cells, B-cells, and/or monocytes. Accordingly, it is expected that the CD147 antigen, can be utilized as a target for the treatment of a variety of diseases. Since the CBL1 antibody has been effective in patients in the treatment of the diseases mentioned above, and based upon the results discussed herein, it is expected that additional CD147 based therapeutics will be similarly effective. Thus, in accordance with the present invention, we have discovered that the molecule CD147 as expressed on certain cells, such as T-cells, B-cells, and/or monocytes, can be utilized for the treatment of a variety of diseases. In particular, we have demonstrated that antibodies that bind to CD147 and that result in the killing of such cells, for example, through the activation of complement, is efficacious in the treatment of diseases. Diseases in which such treatment appears efficacious include, without limitation: graft versus host disease (GVHD), organ transplant rejection diseases (including, without limitation, renal transplant, corneal transplant, and others), cancers (including, without limitation, cancers of the blood (i.e., leukemias and lymphomas), and pancreatic), autoimmune diseases, inflammatory diseases, and others.

As was mentioned above, CBL1 had not previously been indicated to bind to CD147. Further, the particular epitope or antigen to which the CBL1 antibody bound was unknown or at least relatively uncharacterized. Thus, because of the apparent safety and therapeutic efficacy of the CBL1 antibody, we were interested in determining the precise antigen or epitope to which the CBL1 and our ABX-CBL antibody bound. Further, we were interested in further understanding the manner in which the CBL1 antibody was efficacious, particularly in connection with the treatment of GVHD.

By way of reference, the hybridoma line deposited with the ATCC as HB 8214 was not entirely pure. The line produced an IgG antibody and an IgM antibody. Only the IgM is biologically active in inhibition of complement mediated lysis of cells in a mixed lymphocyte reaction assay (MLR). The mechanism of inhibition is via antibody mediated complement-dependent cytotoxicity (CDC) because the inhibition is specific and complement-dependent, as discussed herein. Therefore, in connection with our work described herein, we subcloned the line to produce a cell line producing solely the IgM. Further, the HB 8214 cell line expressing the CBL1 antibody possessed a second kappa light chain (MOPC-21) which appears to have been derived from the myeloma fusion partner, a P3 myeloma cell line, that was used to prepare the original hybridoma cell line. Our subcloned hybridoma cell line possesses and expresses both light chains and the ABX-CBL antibody appears to contain both light chains. IgM antibodies generally possess a pentameric structure, where five heavy and light chain dimers are associated. With the two light chains in the ABX-CBL antibody, we expect that the IgM pentameric structure of the ABX-CBL antibody contains both light chains in various ratios of light chains to form pentamers with homodimeric, heterodimeric, and homo- and heterodimeric combinations.

The role of the MOPC-21 light chain in CBL1 and ABX-CBL binding was unknown. In connection with our work, we endeavored to clarify the role of the MOPC-21 light chain through, for example, preparation of hybridoma subclones that express only the ABX-CBL light chain or the MOPC-21 light chain. One approach that we utilized was to fuise the ABX-CBL hybridoma with a mouse myeloma cell line to achieve light chain shuffling. Upon generation of hybridomas expressing only the MOPC-21 light chain or the ABX-CBL light chain, we were able to conduct certain characterizations to distinguish the role of the two light chains in ABX-CBL binding.

Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection, etc.). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning. A Laboratory Manual (2d ed., Cold Spring Harbor Laboratory Press, Cold Spring Harbor, N.Y. (1989)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences that are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotide linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math 2:482 (1981), by the homology alignment algorithm of Needleman and Wunsch J Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end; the lefthand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions which are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a finctional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (5) confer or modify other physicochemical or fuinctional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to a CD147, under suitable binding conditions, (2) ability to modify CD147's binding to its ligand or receptor, or (3) ability to kill or inhibit growth of CD]47 expressing cells in vitro or in vivo. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986); Veber and Freidinger TINS p.392 (1985); and Evans et al. J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids, sugar, or other carbohydrate side chains and usually have specific three dimensional structural characteristics, as well as specific charge characteristics. An antibody is said to specifically bind an antigen when the dissociation constant is ≦1 μM, preferably ≦100 nM and most preferably ≦10 nM.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinylated moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

The term “substantially non-toxic to resting T-cells and resting B-cells” as used herein means, preferably, that the antibody in the presence of compliment at at least a 2-fold lower level of depletion of resting cells occurs than the level of depletion of activated T- and B-cells. More preferably, there is at least a 5-fold lower level of cell depletion of resting cells compared to the level of depletion of activated cells. And, most preferably, there would be no detectable depletion of resting cells.

ABX-CBL Antigen Identification and Characterization

We undertook two primary approaches to the identification and characterization of the antigen to which the ABX-CBL antibody bound (i) an immunoaffinity purification approach and (ii) a classical protein purification approach.

Immunoaffinity Purification

We investigated immunoaffinity purification of the antigen to which the CBL1 antibody bound. The antigen to which the CBL1 antibody bound appeared to be highly expressed on CEM cells which is a T lymphoblastoid cell line derived by Foley et al. Cancer 18:522-529 (1965) and available from the ATCC, Rockville, Md. (ATCC No. CCL-119). Immunoaffinity purification using the native ABX-CBL antibody was frustrated by the fact that the ABX-CBL antibody is an IgM antibody having a pentameric structure and prone to nonspecific interactions in vitro. Therefore, we prepared human IgG2 antibodies against CEM cells and tested for competition with the ABX-CBL antibody in binding assays with CEM cells. Such human antibodies were prepared in accordance with Mendez et al. Nature Genetics 15:146-156 (1997) and U.S. patent application Ser. No. 08/759,620, filed Dec. 3, 1996, the disclosures of which are hereby incorporated by reference herein in their entirety, through the immunization of XenoMouse™ animals with CEM cells, followed by fusions, and screening of the resulting hybridoma supernatants against CEM cells and in FACS competition assays with the ABX-CBL antibody. In the FACS competition assays, inhibition of the binding of ABX-CBL antibodies, labeled with FITC, to CEM cells was analyzed, both alone and in the presence of hybridoma supernatants containing human antibodies reactive with the CEM cells.

Four hybridoma clones were isolated and determined, in this manner, to be that were highly competitive with the ABX-CBL antibody in binding to the CEM cells. One hybridoma clone, designated 2.6.1, was selected for further analysis. We generated ascites to each of the hybridomas, including the 2.6.1 hybridoma, in SCID mice and purified the 2.6.1 antibody using a Protein A affinity purification process using standard conditions. From the purified 2.6.1 antibody, we prepared an immunoaffinity column. To prepare the column, the purified 2.6.1 antibody was conjugated to CNBr activated Sepharose-4B, according to the manufacturer's specifications. Approximately 8.4 mg of the antibody was conjugated to about 2.0 g of the activated Sepharose. We passed cell lysates of CEM cells through the column and eluted the components that bound. The elution product was analyzed by Western blotting and probing with both the ABX-CBL antibody and the 2.6.1 antibody. Based upon preliminary data, the 2.6.1 antibody bound most intensely to a molecule or molecules contained within a diffuse band from about 45-55 KD, while the ABX-CBL antibody showed binding with a low intensity to a similar diffuse band from about 45-55 KD. Through use of preparative gel electrophoresis and electroblotting techniques, we isolated a portion of the 45-55 KD band and obtained a partial amino acid sequence of the molecule (35/40 residues). The resulting sequence information was analyzed through a protein database search (Protein Identification Resourse (PIR) R47.0, December 1995) and the sequence comparison data indicated that the molecule was CD147.

Protein Purification and Sequencing

In connection with our work related to the characterization of the antigen to which the ABX-CBL antibody bound, we saw significant ABX-CBL binding on Western blots to molecules localized in relatively sharp bands at 35 KD and 62 KD. The intensity of this 35 KD band appeared to vary from prep to prep, depending on culture age and other conditions not completely understood. Therefore, we initially purified the 62 KD material. Because the N-terminus was blocked, we cleaved the protein with CNBr and sequenced two of the peptides that resulted from the cleavage. The resulting sequence information was analyzed through a protein database search (Protein Identification Resourse (PIR) R47.0, December 1995) and the sequence comparison data indicated that the molecule was heterogeneous ribonuclear protein k (hnRNP-k). Such molecule is an intracellular component, and, accordingly, does not conform to the observations that the ABX-CBL antibody appeared to recognize an extracellular component. Nevertheless, the identification of this molecule may be useful in connection with further understanding of the binding of ABX-CBL to CD147, for example in connection with epitope elucidation.

Characterization of the 35 KD band can also be undertaken for similar reasons. In such an approach, the 35 KD molecule can be purified in a similar manner to that utilized in connection with the 62 KD band mentioned above. The purified material from the 35 KD band can be characterized to further understand any potential structural differences between material contained in the 45-55 KD CD147 band. The material contained in the 35 KD band can be sequenced to either demonstrate that the material is CD147 or to determine epitopic information related to ABX-CBL's binding to CD147.

Further Elucidation of CD147 Binding and Epitopic Analysis of ABX-CBL

As was discussed above, another area of exploration is in connection with the elucidation of the binding of the ABX-CBL antibody to the CD147 molecule. Because of the safety and efficacy of the ABX-CBL antibody, we expect that molecules, particularly antibodies, that mimic the binding of the ABX-CBL antibody to CD147 should possess a similar safety profile. Thus, in order to further understand the binding of the ABX-CBL antibody to CD147, we have undertaken, or designed, experiments in order to elucidate the same. Our experiments include (i) cloning of CD147 and expression in eukaryotic (COS) cells, (ii) expression in prokaryotic (E. coli) cells, and (iii) screening of random peptide libraries utilizing phage display techniques.

Cloning of CD147 and Expression in COS Cells

We cloned CD147 cDNA from a Jurkat library (Stratagene), prepared constructs for transfection, and transfected COS cells with the CD147 cDNA. Transfected cells were analyzed for expression of CD147 utilizing FACS analysis and Western blotting in connection with the ABX-CBL antibody, the 2.6.1 antibody, and the Pharmingen antibody mentioned above. COS cells transfected with CD147 cDNA showed binding to each of the antibodies in each of the FACS and Western blot analyses. In contrast, COS cells transfected with control vectors were negative for binding with each of the 2.6.1 and ABX-CBL antibodies. With respect to the Pharmingen antibody, certain background staining was observed in cells transfected with control vectors on FACS and no binding on Western blot analysis. The transfected cells showed significant binding over background on FACS and were positive on Western blot analysis. Our results confirm that the ABX-CBL and the 2.6.1 antibodies bind to CD147.

Expression of CD147 in E. Coli Cells

Utilizing a slightly modified vector, we also transfected E. coli cells with the CD147 cDNA. The E. coli cells so transfected were capable of expression of the CD147 molecule as evidenced by Western blotting analysis of each of the ABX-CBL, 2.6.1, and Pharmingen antibodies. Since the prokaryotic E. coli cells should not glycosylate the expressed CD147, it was expected that the molecular weight of the CD147 expressed by the E. coli should closely approximate the predicted, unglycosylated molecular weight of CD147 of about 27 KD. Indeed, in each case, binding of the three antibodies on Western blot analysis was observed to a band between about 27 and 30 KD.

This data further confirms that the ABX-CBL and the 2.6.1 antibodies bind to CD147. Further, the evidence indicates that ABX-CBL binding to CD147 is not directly based on carbohydrate binding, i.e., that ABX-CBL does not bind directly to a carbohydrate epitope on CD147. Such data, however, does not eliminate the possibility that binding to CD147 is influenced by the presence of carbohydrate or glycosylation.

Screening Utilizing Phase Display

In order to further elucidate the binding of the ABX-CBL antibody to CD147, we undertook phage display experiment. Such experiments were conducted through panning a phage library expressing random peptides for binding with the ABX-CBL and 2.6.1 antibodies to determine if we could isolate peptides that bound. If successfiul, certain epitope information can be gleaned from the peptides that bind.

In general, the phage libraries expressing random peptides were purchased from New England Biolabs (7-mer and 12-mer libraries, Ph.D.-7 Peptide 7-mer Library Kit and Ph.D.-12 Peptide 12-mer Library Kit, respectively) based on a bacteriophage M13 system. The 7-mer library represents a diversity of approximately 2.0×10⁹ independent clones, which represents most, if not all, ofthe 20⁷=1.28×10⁹ possible 7-mer sequences. The 12-mer library contains approximately 1.9×10⁹ independent clones and represents only a very small sampling of the potential sequence space of 20¹²=4.1×10¹⁵ 12-mer sequences. Each of 7-mer and 12-mer libraries were panned or screened in accordance with the manufacturer's recommendations in which plates were coated with an antibody to capture the appropriate antibody (goat anti-human IgG Fc for the 2.6.1 antibody and goat anti-mouse 4 chain for the ABX-CBL antibody) followed by washing. Bound phage were eluted with 0.2 M glycine-HCl, pH 2.2. After 3 rounds of selection/amplification at constant stringency (0.5% Tween), through use of DNA sequencing, we characterized a total of 5 clones from the 7-mer library and 6 clones from the 12-mer library reactive with the ABX-CBL antibody and a total of 6 clones from each of the 7-mer and 12-mer libraries reactive with the 2.6.1 antibody. Reactivity of the peptides was determined by ELISA. For an additional discussion of epitope analysis of peptides see also Scott, J. K. and Smith, G. P. Science 249:386-390 (1990); Cwirla et al. PNAS USA 87:6378-6382 (1990); Felici et al. J. Mol. Biol. 222:301-310 (1991), and Kuwabara et al. Nature Biotechnology 15:74-78 (1997).

No consensus sequence was readily apparent for reactivity of the 2.6.1 antibody with CD147. However, sequence alignment of the characterized 7-mer and 12-mer sequences against the amino acid sequence of CD147 yielded a number of matches for a single sequence within CD147 from residue number 177 through residue number 188 (ITLRVRSH (SEQ ID NO: 1)). In particular, each of the 7-mers contained sequence matches (represented by *) to 3 or more residues within this sequence of CD147:

7-mer sequences

| | | |
|---|---|---|
| | * * * | |
| 1. | EE RLR S Y | (SEQ ID NO:2) |
| | *** | |
| 2. | YE RVR W Y | (SEQ ID NO:3) |
| | * * * | |
| 3. | EE RLR S Y | (SEQ ID NO:4) |
| | ** * | |
| 4. | AE RIR S I | (SEQ ID NO:5) |
| | * * * | |
| 5. | EE RLR S Y | (SEQ ID NO:6) |

Further, 4 of the 12-mers contained sequence matches (represented by *) to 3 or more residues within this sequence of CD147, with 4 matches for 12-mer peptide number 1 and for 6 matches of 12-mer peptide number 2:

12-mer sequences

| | | |
|---|---|---|
| | * * * * | |
| 1. | TVHGDL RLR S LP | (SEQ ID NO:7) |
| | * * * * * * | |
| 2. | TNDIGL RQR S HS | (SEQ ID NO:8) |
| | * * * | |
| 3. | SPLLDGQ RER S Y | (SEQ ID NO:9) |
| | * * * | |
| 4. | YDLPM RSR S YPG | (SEQ ID NO:10) |

These results indicate a consensus sequence of RXRS (SEQ ID NO:11) that is present in 10 of the sequenced clones. Accordingly, we had a synthetic peptide prepared (AnaSpec Incorporated, San Jose, Calif.) which spanned residues 169-183 of CD147 with the following sequence (with —OH representing carboxy terminus):

| KGSDQAIITLRVRSH-OH | (SEQ ID NO:12) |
|---|---|
| \| \| | |
| 169 184 | |

Below, the amino acid sequence of CD147 is provided with the 15-mer peptide's sequence indicated by double underlining and the RXRSH (SEQ ID NO:13) consensus sequence indicated in bold. In addition, putative N-linked glycosylation sites of CD147 are shown as underlined and italics:

CD147 Sequence

(SEQ ID NO:14)

MAAALFVLLGFALLGTHGASGAAGTVFTTVEDLGSKILLTCSLNDSATEV
TGHRWLKGGVVLKEDALPGQKTEFKVDSDDQWGEYSCVFLPEPMGTANIQ
LHGPPRVKAVKSSEHINEGETAMLVCKSESVPPVTDWAWYKITDSEDKAL
MNGSESRFFVSSSQGRSELHIENLNMEADPGQYRCNGTSSKGSDQAIITLR
VRSHLAALWPFLGIVAEVLVLVTIIFIYEKRRKPEDVLDDDDAGSAPLKS
SGQHQNDKGKNVRQRNSS

The 15-mer peptide was assayed using ELISA and it was determined that the ABX-CBL antibody specifically bound to the peptide. Further, neither the 2.6.1 antibody nor a control murine IgM antibody bound to the peptide. However, based on a competition study between the CD147 antigen and the 15-mer peptide, the ABX-CBL antibody's binding to the 15-mer peptide can only be measured when the 15-mer peptide is coated on plates and not when the peptide is in solution. Indeed, in competition experiments in which the ABX-CBL antibody is bound to either the peptide or the CD147 antigen coated to plates, the ABX-CBL antibody is not removed or replaced by the peptide in solution even at high concentrations. Nevertheless, the binding of the ABX-CBL antibody to the 15-mer peptide can be specifically competed by the CD147 antigen and positive phage preparations mentioned above but not with non-specific antigen (i.e., L-Selectin isolated from cell membrane or human plasma) or the negative phage preparations mentioned above. Similarly, the binding of the ABX-CBL antibody to the CD147 antigen can be specifically competed by positive phage preparations as compared to negative phage preparation in competition assays using preincubation.

These results indicate that while the sequence within CD147 that contains the consensus sequence RXRSH is important to the binding of the ABX-CBL antibody to CD147, it does not fully explain ABX-CBL's binding to CD147. Indeed, the data also suggests that the consensus sequence contained either in the 15-mer peptide when bound to the plate or the reactive phage materials when tethered to the phage coat protein binds more tightly to the ABX-CBL antibody than does the free peptide in solution. Taken together, while not wishing to bound to any particular theory or mode of operation, it is possible that CD147 possesses certain conformations that are not well mimicked in the 15-mer peptide in solution. Nevertheless, the above epitopic information is important to understanding the manner in which the ABX-CBL antibody binds to CD147 and to producing other candidate molecules against CD147 as a therapeutic target.

It is interesting to note that in addition to the results above in connection with the presence of the RXRSH consensus sequence within CD147, we also looked for the presence of the consensus sequence within the hn-RNP-k protein to which ABX-CBL also appears to bind. Such analyses were conducted by sequence alignment against the phage derived peptides discussed above. Two sequences were found which possessed statistically interesting matches:

First, there was a match (indicated by *) of 5 amino acids with the 7-mer peptide number 4:

| * ** ** | |
|---|---|
| PE RIL SI | (SEQ ID NO:15) |
| \| | |
| 84 | |

Second, there was a match (indicated by *) of 5 amino acids with the 12-mer peptide number 1:

| * * * ** | |
|---|---|
| GGS RAR NLP | (SEQ ID NO:16) |
| \| \| | |
| 300 306 | |

The amino acid sequence of the hn-RNP-k protein is provided below with such sequences indicated by double underlining. In addition, a number of RXR sequence motifs are present in the hn-RNP-k protein's sequence which are also indicated by underlining:

hn-RNP-k Protein Sequence

(SEQ ID NO:17)

METEQPEETFPNTETNGEFGKRPAEDMEEEQAFKRSRNTDEMVELRILLQ
SKNAGAVIGKGGKNIKALRTDYNASVSVPDSSGPERILSISADIETIGEI
LKKIIPTLEEGLQLPSPTATSQLPLESDAVECLNYQHYKGSDFDCELRLL
IHQSLAGGIIGVKGAKIKELRENTQTTIKLFQECCPHSTDRVVLIGGKPD
RVVECIKIILDLISESPIKGRAQPYDPNFYDETYDYGGFTMMFDDRRGRP
VGFPMRGRGGFDRMPPGRGGRPMPPSRRDYDDMSPRRGPPPPPPGRGGRG
GSRARNLPLPPPPPPRGGDLMAYDRRGRPGDRYDGMVGFSADETWDSAID
TWSPSEWQMAYEPQGGSGYDYSYAGGRGSYGDLGGPIITTQVTIPKDLAG
SIIGKGGQRIKQIRHESGASIKIDEPLEGSEDRIITITGTQDQIQNAQYL
LQNSVKQYSGKIFF

Without wishing to be bound to any particular theory or mode of operation, it is possible that the binding of the ABX-CBL antibody to the hn-RNP-k protein is partially explained by the presence of these motifs within the protein.

Discussion of Results of Antigen Identification and Analysis

It is interesting to note that the ABX-CBL antibody appears to bind to the 45-55 KD band with less intensity than it does the 35 KD band in CEM cell lysates. However, without wishing to be bound to any particular theory or mode of operation of the ABX-CBL antibody, the 35 KD band could either represent another epitope or could be an alternative form of CD147. Indeed, as discussed above, there is evidence in the literature for alternative splicings of CD147 or differential glycosylation. See e.g., Kanekura et al. Cell Struct Funct 16:23-30 (1991) (Basigin); Schlosshauer B Development 113:129-140 (1991) (Neurothelin); Fadool J M & Linser P J J Neurochem 60:1354-136 (1993) (5A11/HT7); Nehme et al. J Cell Biol 120:687-694 (1993) (CE9); DeCastro et al. J Invest Dennatol 106:1260-1265 (1996) (EMMPRIN); Spring et al. Eur J Immunol 27:891-897 (1997) (Ok^{a}). Anecdotal evidence indicates that a 35 KD band could correspond to a singly-glycosylated form of CD147. See Kanekura et al. Cell Struct Funct 16:23-30 (1991). Further, it is also interesting to note that in comparisons of Western blots produced by two commercially available anti-CD147 antibodies (RDI-CBL535 (an anti-CD147 IgG2 antibody), available from RDI, Flanders, N.J., and 36901A (an anti-CD147 IgG1 antibody), available from Pharmingen, San Diego, Calif.) to the ABX-CBL and 2.6.1 antibodies indicates that each of the commercially available antibodies recognize a molecule that has a molecular weight around 35 KD and appearing similar to the 35 KD band recognized by the ABX-CBL antibody. However, the 45-55 KD diffuse band is more intense. See FIG. 1.

Based upon preliminary data, another interesting observation is that in the immunoaffinity purification mentioned above, when the effluent product from the 2.6.1 antibody was probed with the ABX-CBL antibody, the 35 KD band was no longer visible by Western blot. Rather, the ABX-CBL antibody appeared to bind to the diffuse band from 45-55 KD with relatively low intensity.

Further, our results in phage display experiments indicates that the ABX-CBL antibody and the 2.6.1 antibody bind to different epitopes. However, from our work related to the expression of CD147 in E. coli cells and based on the phage display work, the ABX-CBL antibody appears to recognize a protein epitope of CD147 and glycosylation, alone, does not appear responsible for ABX-CBL binding to CD147.

Nevertheless, in light of all of the foregoing, taken together, our results and data indicate that the ABX-CBL antibody does bind to the CD147 antigen. However, the ABX-CBL antibody appears to preferentially recognize a different epitope than recognized by the 2.6.1 or commercially available antibodies. Our finding that the ABX-CBL antibody binds to the CD147 antigen is indicative that a form of CD147 as expressed on particular cells is a viable therapeutic target for the treatment of disease.

Functional Understanding of the Mode of CD147 Therapy

As mentioned above, the CBL1 antibody has been used extensively in the treatment of GVHD in patients. Indeed, about a number of GVHD patients have been treated using the CBL1 antibody with a high percent success rate. Corneal and renal transplant studies have shown similar efficacy. Further, no signs of safety concerns or adverse effects have been observed. This is striking, given that, as discussed above, CD147 is so widely expressed in various tissues and cells of man. One would be concerned that an antibody to CD147 could cause a variety of adverse effects. Accordingly, we also endeavored to study the mechanism through which the CBL1 antibody operated to result in the treatment of disease, focused on models relevant to the reversal of GVHD. Understanding the mechanism could assist in elucidating why the CBL1 antibody is efficacious in patients and could also provide an understanding of how to use the antigen to which the CBL1 antibody binds, CD147, in the treatment of disease.

There are several possible explanations related to the safety and specificity of the CBL1 antibody in the treatment of disease. Without limitation, these include (i) that there is a unique role of complement mediated cell killing (complement dependent cytotoxicity, CDC), (ii) that certain cells in becoming activated become sensitive to CBL1 binding and cell killing, (iii) that there are particular protective elements in certain cellular populations that render the cells resistant to CBL1 induced CDC, (iv) that CD147 expression levels are higher in given populations of cells (which could also be relevant to CDC), and (v) that the CBL1 antibody binds to a particular form of CD147 expressed on certain cellular populations (as discussed above). Each of these roles will be discussed in additional detail below.

Complement Mediated Killing of Cells

The role of complement mediated cell killing (complement dependent cytotoxicity, CDC) in connection with the CBL1 antibody has been studied previously and we have additionally studied its role extensively.

Past Work with CBL1

The UCLA group mentioned above (see e.g., U.S. Pat. Nos. 5,330,896 and 5,643,740) provided certain evidence that the CBL1 antibody operated through killing of certain activated cell populations while the antibody did not react with non-activated cells. For example, in microcytotoxicity assays, the CBL1 antibody was disclosed to kill activated lymphocytic cells but not non-activated lymphocytic or other normal cells. Further, the patents disclose that the cell killing operated through complement mediated killing of the cells.

Further Demonstration of the Role of CDC

Indeed, in our work, we have further demonstrated that CBL1 and ABX-CBL operates through complement mediated cell killing. We have utilized a mixed lymphocyte reaction (MLR) assay or a modified MLR assay in our work. The MLR assay provides an in vitro system for assaying proliferation of alloreactive T-lymphocytes. In this manner, the MLR assay is an excellect model of GVHD in patients receiving bone marrow transplant (BMT). In the MLR assay, MHC mismatch lymphocytes from two individuals are co-cultured. Typically the assays are set up so that the lympocytes from one patient are inactivated by, for example, radiation (the “stimulators”) and the lymphocytes from the other patient are able to act as “Responders” and proliferate and undergo extensive blast transformation. After a suitable period of co-culture, the extent of proliferation of the cells can be quantified by adding tritium-labeled thymidine ([³H] thymidine) to the culture medium and monitoring uptake of the label into the DNA of the Responder lymphocytes.

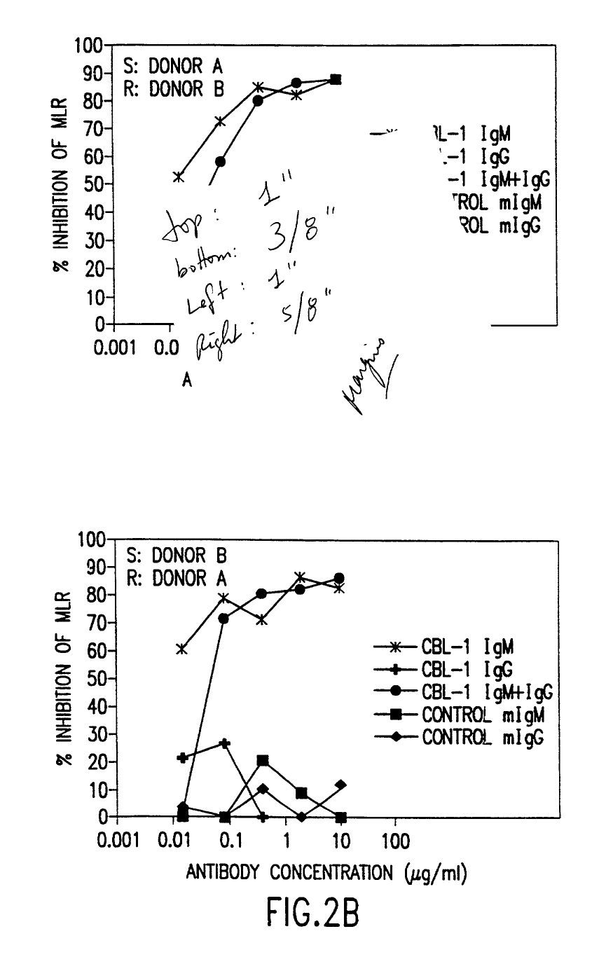

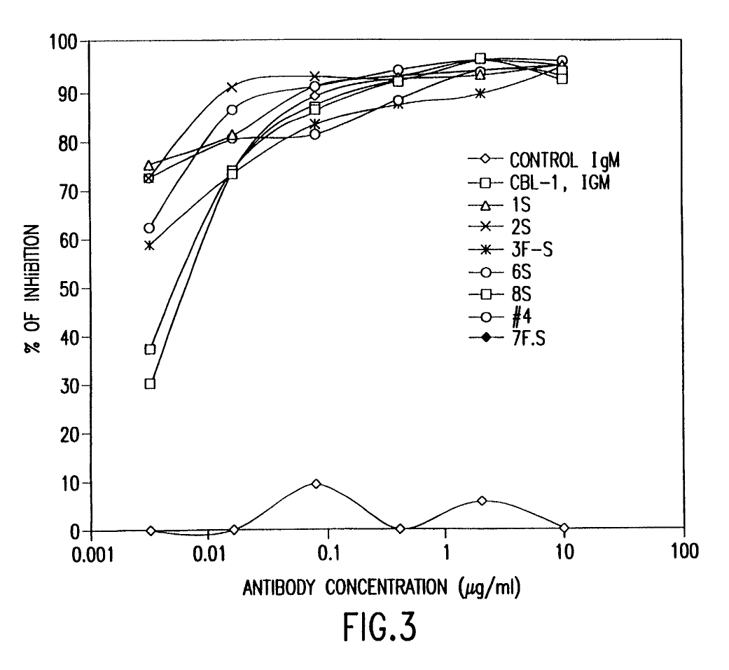

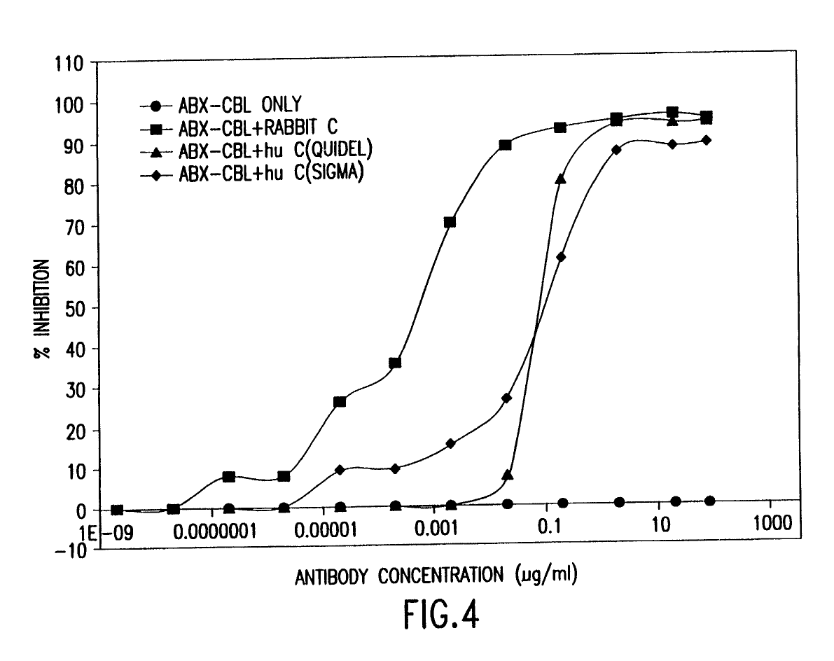

In our work, use of the CBL1 antibody by itself, the isotype-matched control mouse IgM antibody by itself (FIG. 2), or complement (either human or rabbit) by itself in an MLR or ConA induced lymphocyte proliferation assay is ineffective in inhibiting T-cell proliferation. See FIGS. 2-5. However, when both complement and the CBL1 and/or ABX-CBL antibody are present, T-cell proliferation is inhibited in a dose dependent manner. See FIGS. 2-5. The human IgG2 antibody 2.6.1 is ineffective in inhibiting T-cell proliferation in the same assay, either by itself, or in combination with complement. See FIG. 5. This is expected, since the 2.6.1 antibody as a gamma-2 isotype is notoriously less efficient in complement mediated lysis than is an IgM antibody, such as the CBL1 or ABX-CBL antibody.

Role of Cellular Activation Levels

We have also studied whether certain cells in becoming activated become sensitive to ABX-CBL binding and cell killing.

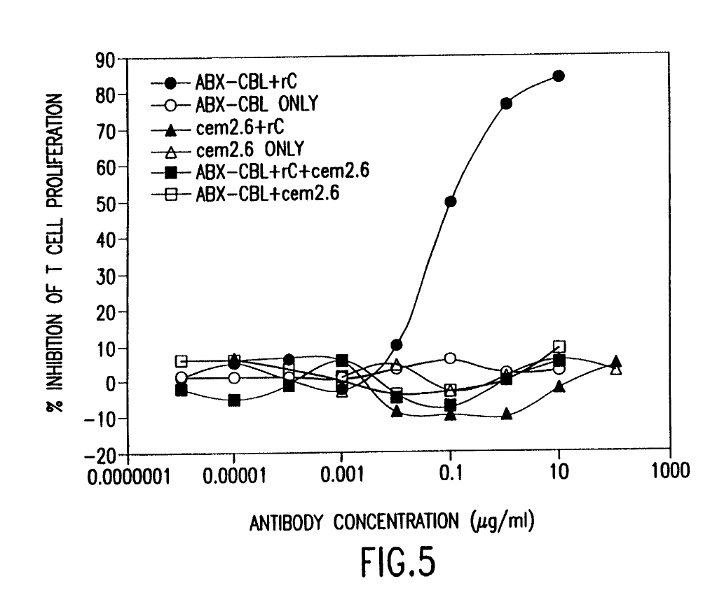

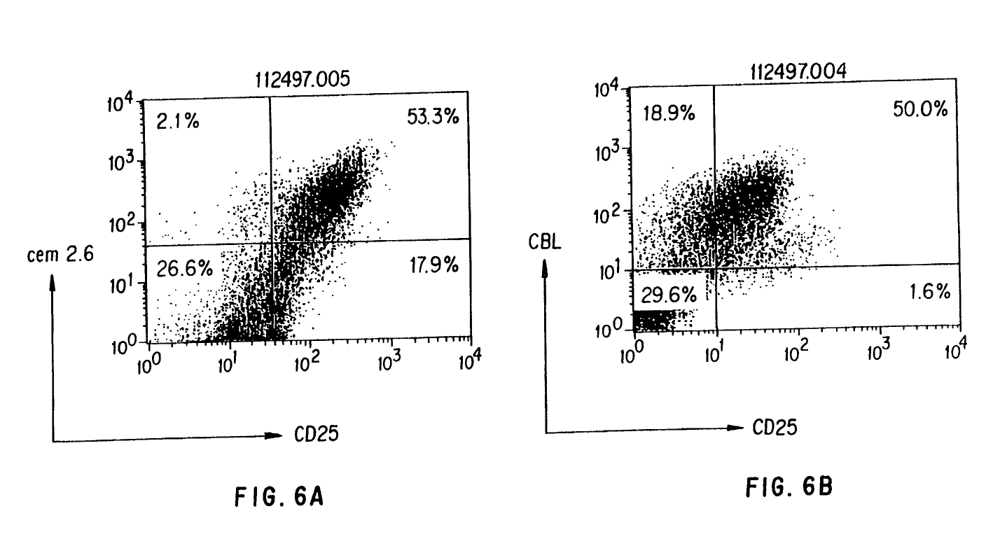

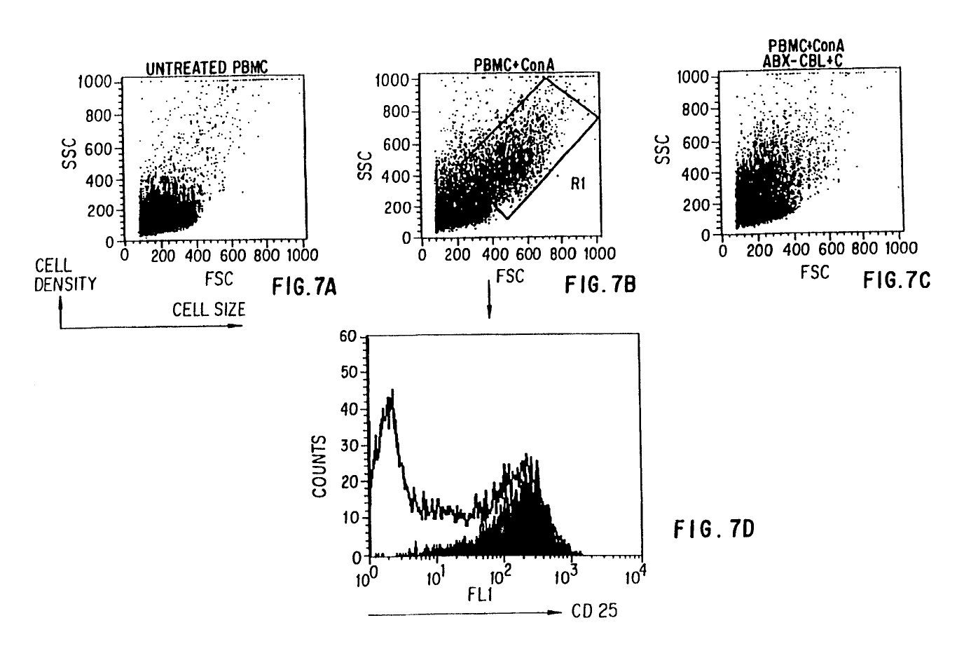

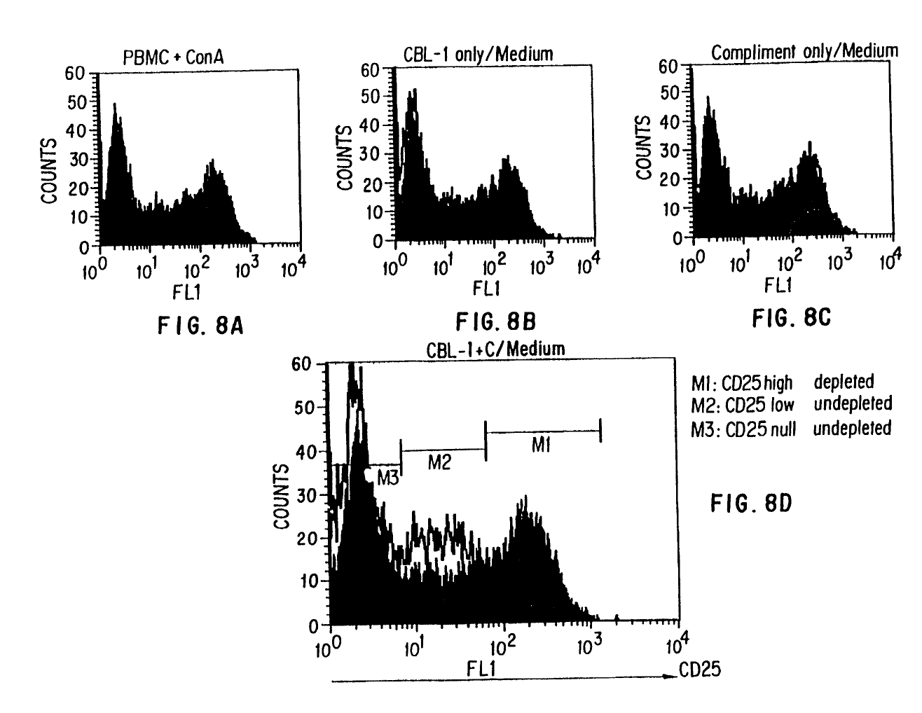

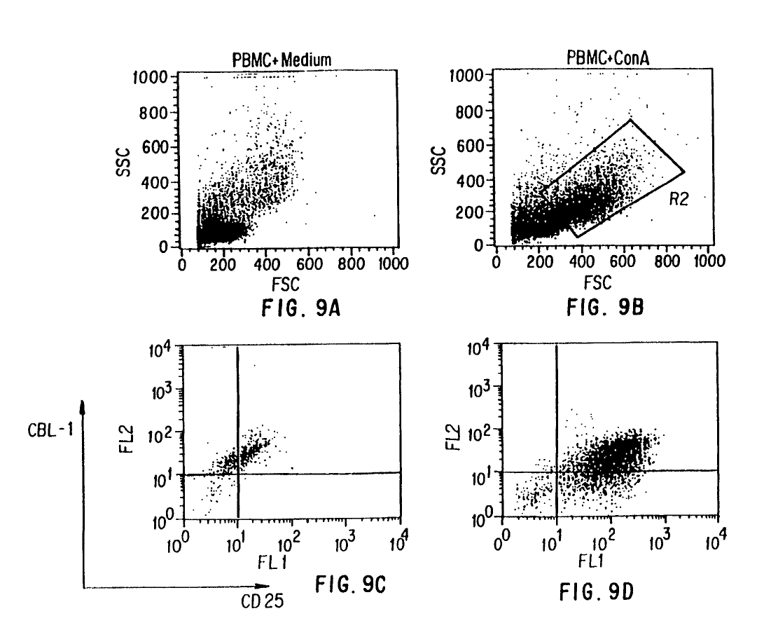

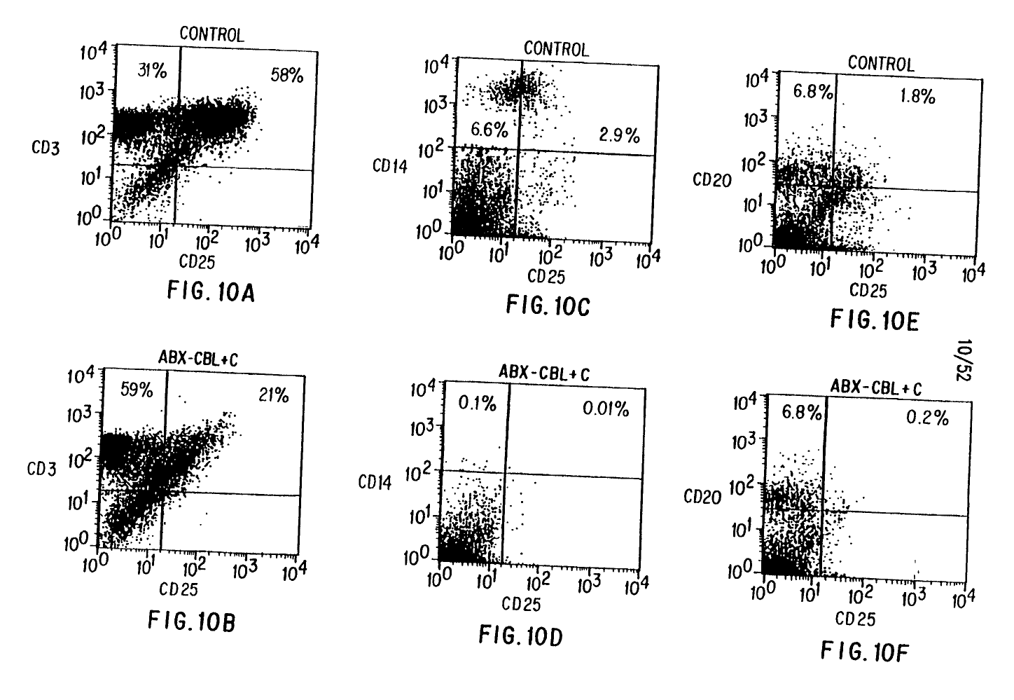

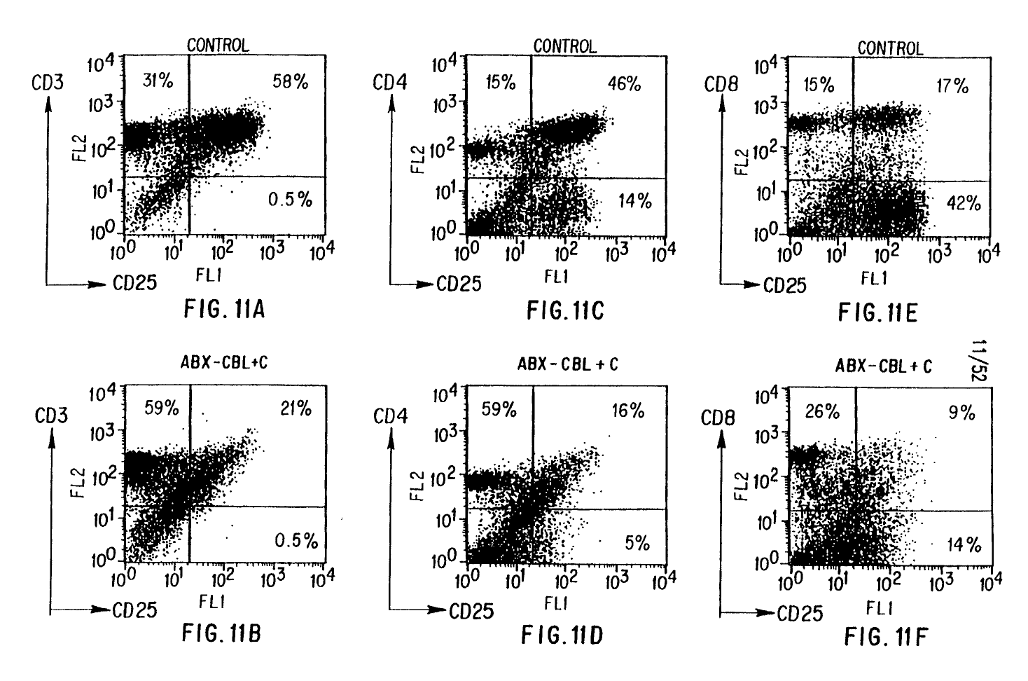

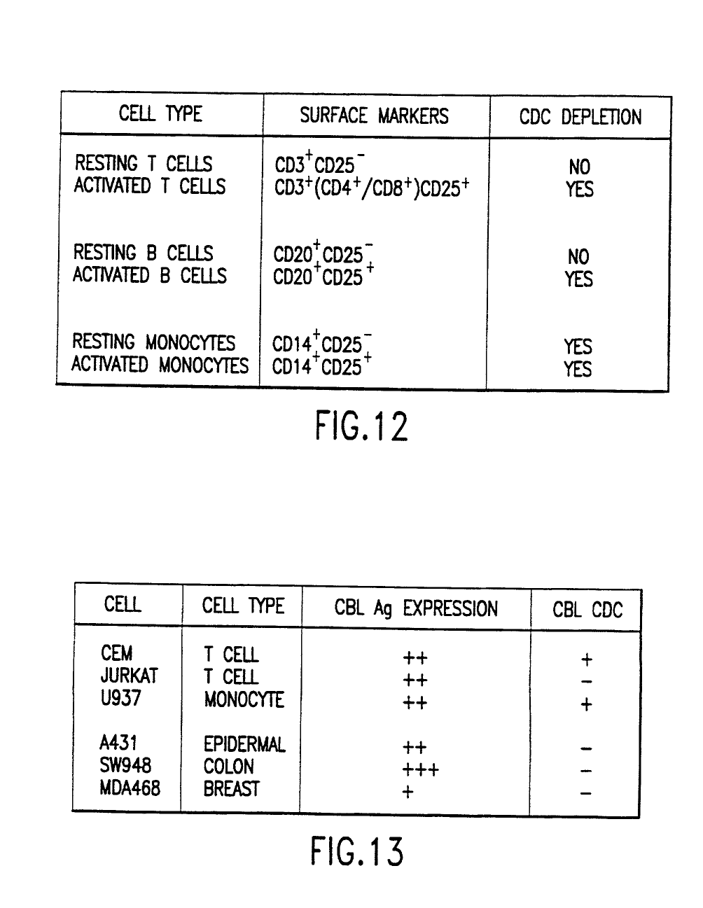

Indeed, we have demonstrated in our work that the T-cell activation marker, CD25 (the alpha-2 subunit of the IL-2 receptor), appears to be expressed in high levels in the same cellular populations as those expressing the antigen to which the ABX-CBL antibody binds. See FIG. 6. This finding provided a useful marker to detect whether activated cells were depleted in connection with the MLR assay. Where the MLR assay is conducted utilizing ABX-CBL alone, complement alone, or ABX-CBL and complement in combination, it is only in those experiments where ABX-CBL and complement are used in combination that CD25 expressing cell populations are depleted. See FIGS. 7-11. In particular, FIG. 8 shows cells expressing low levels of CD25. The selective killing of different cell populations are shown in FIGS. 10-12.

Role of Density or Exression Levels of CD147 in CDC

We have also considered whether CD147 expression levels are higher in given populations of cells (which could also be relevant to CDC).

In flow cytometry studies with peripheral blood mononuclear cells (PBMC) with the ABX-CBL antibody, we have noticed that, prior to the addition of complement, there are populations of cells that appear to express high and low levels of CD147. After complement is added, there are populations of cells that appear to correspond to the low level expressers mentioned above. It appears that these results could be indicative of density of CD147 expression levels on the cell surface. Density can play a role in CDC through providing additional antigen binding sites to allow for distortion of the antibody which is the first step in triggering the complement cascade. Upon distortion of the antibody, the factor clq binds first and the cascade proceeds.

Whether the expression level (or, density) of CD147 in cellular populations plays a role in the therapeutic efficacy of the ABX-CBL antibody can be assayed through analyzing the expression levels of the CD147 molecule in various cellular populations. Generally, the experiments are conducted where beads having various known quantities of the CD147 antigen on their surface are prepared and analyzed on FACS (i.e., utilizing a FITC-labeled anti-CD147 IgG antibody) in order to generate approximately 10-20 data points of different quantities of antigen on the beads. A linear regression curve is prepared from such data. Thereafter, cells expressing the CD147 antigen can be run through FACS and the relative quantities of antigen on the surface of the cells can be calculated from the linear regression curve.

Presence and Role of Protective Elements in Cellular Populations

We have also studied whether there is a correlation between certain cellular protective elements in particular cellular populations that inhibit CDC induced by ABX-CBL binding and fixing of complement.

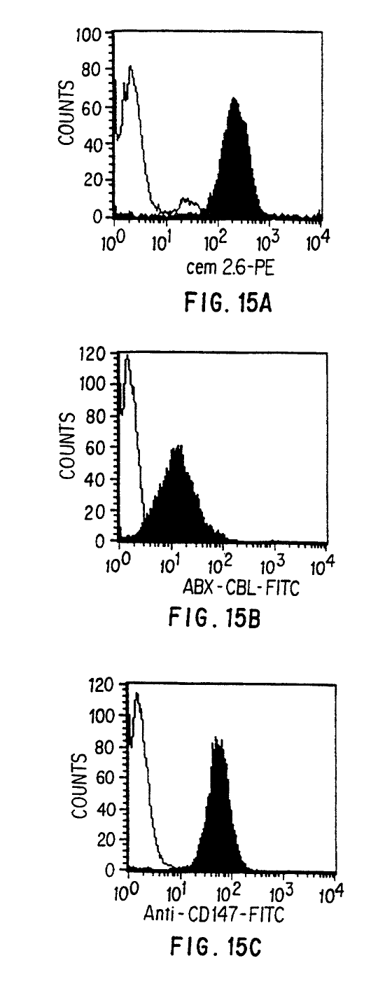

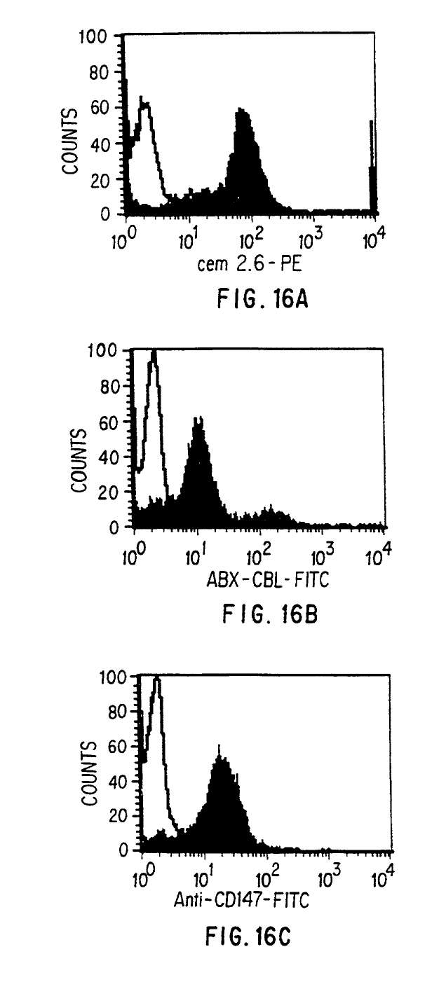

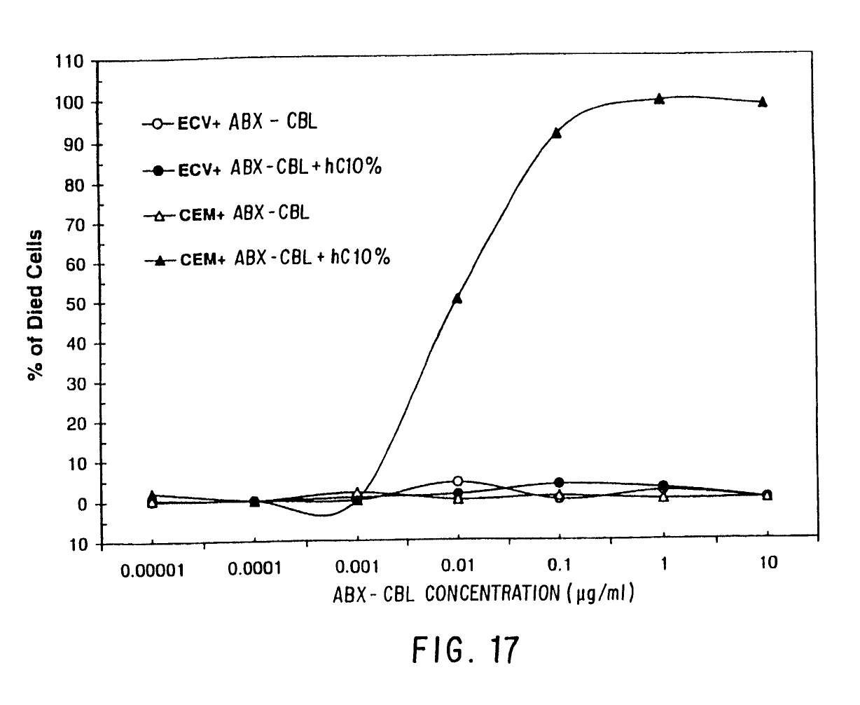

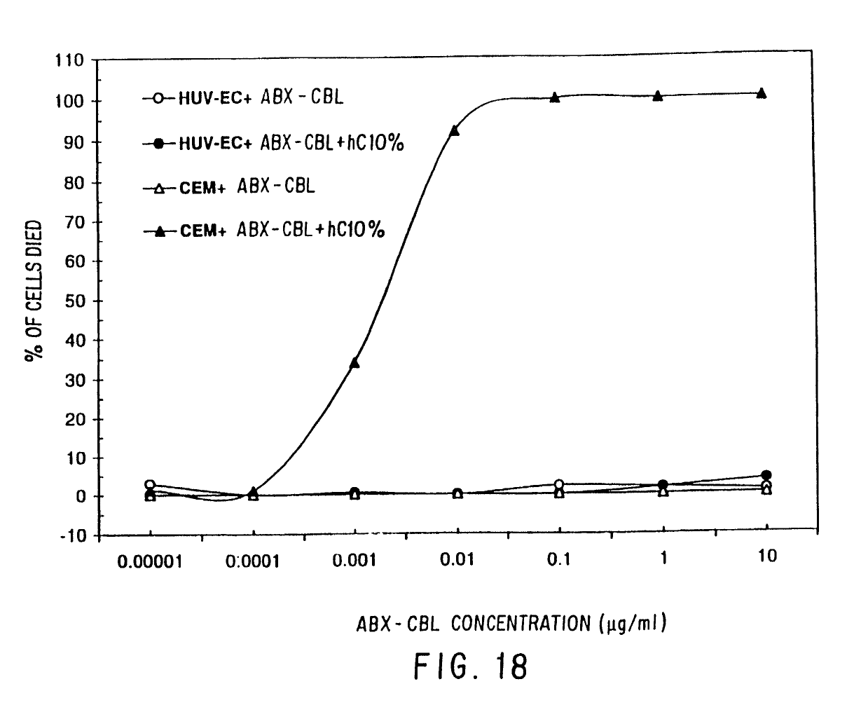

In connection with this work, we have investigated various cells to which the ABX-CBL antibody binds and considered whether such cells were (i) killed and (ii) if so, was the mechanism similar to complement mediated lysis. In the experiment, we looked for ABX-CBL antibody binding to a number of cells (and, thus, the antigen to which the ABX-CBL antibody binds is expressed upon such cells). Those cells to which ABX-CBL would bind were then tested for complement mediated lysis through treatment with the ABX-CBL antibody and complement. Two T-cell lines (CEM and Jurkat cells), a monocyte line (U937 cells), and three tumor cell lines (A431 (epidermal), SW948 (colon), and MDA468 (breast)), each of which bound the ABX-CBL antibody were examined. Despite the expression on such cells lines, the ABX-CBL antibody is very specific about which cells are killed, being restricted to the CEM T-cell line and U937 monocyte line. See FIG. 13. We also analyzed two endothelial cell lines (i) ECV-304 (ATCC CRL-1998) is a spontaneously transformed immortal EC established from the vein of an apparently normal human umbilical cord and carrying EC characteristics and (ii) HUVEC-C (ATCC CRL-1730) is an EC line derived from the vein of a normal human umbilical cord. Using FACS, we found that the ECV-304 and HUVEC-C lines each stained positive against the 2.6.1, Pharmingen, and ABX-CBL antibodies suggesting that these ECs do express CD147 on the surface. FIGS. 15 and 16, respectively. We then carried out in vitro Alamar-blue based CDC assay and demonstrated that both EC lines were resistant to ABX-CBL mediated CDC in the presence of human complement. See FIGS. 17 and 18, respectively.

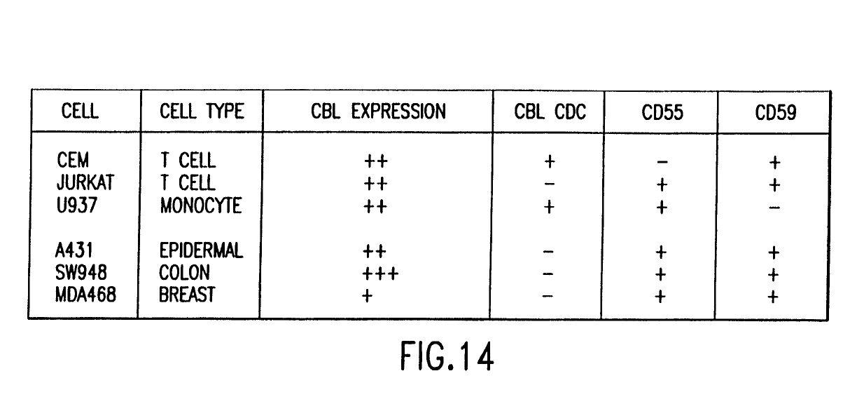

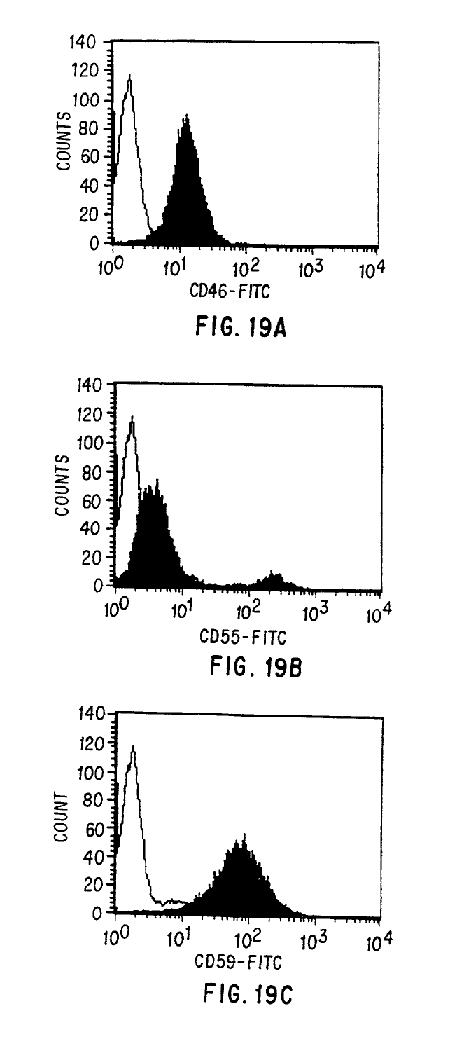

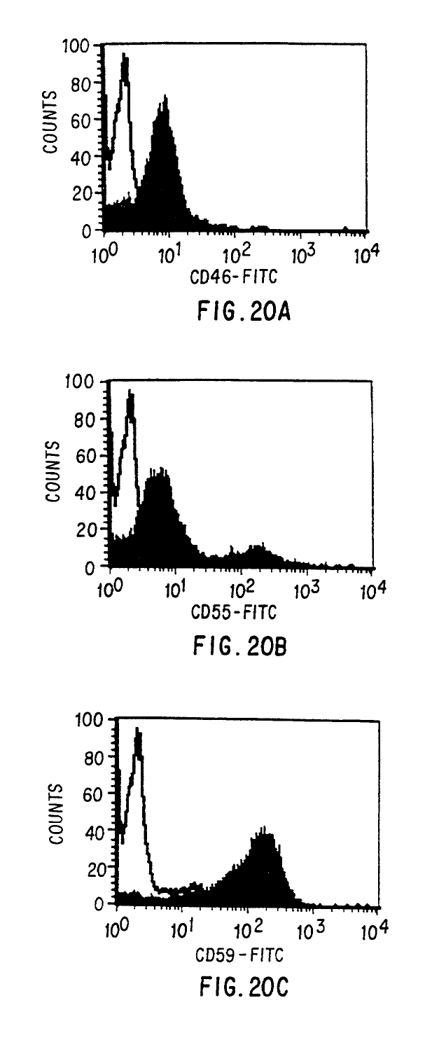

In order to further understand why cells that all appear to express CD147 would not be killed by the ABX-CBL antibody in the presence of complement, we looked into CD46, CD55, and CD59 expression in such cells. Each of CD46 (membrane cofactor protein, MCP), CD55 (decay accelerating factor, DAF), and CD59 (membrane attack complex inhibitor, MACI) have been implicated as complement inhibitory molecules. See e.g., Liszewski et al. Annu. Rev. Immunol. 9:431 (1991) and Loveland et al. “Coordinate functions of multiple complement regulating molecules, CD46, CD55 and CD59” Transpl. Proc. 26:1070 (1994) related to CD46, Kinoshita et al. “Distribution of decay-accelerating factor in the peripheral blood of normal individuals and patients with paroxysmal nocturnal hemoglobinuria” J. Exp. Med. 162:75 (1985) and Loveland et al. “Coordinate functions of multiple complement regulating molecules, CD46, CD55 and CD59” Transpl. Proc. 26:1070 (1994) related to CD55, and Whitlow et al. “H19, a surface membrane molecule involved in T-cell activation, inhibits channel formation by human complement” Cell. Immunol. 126: 176 (1990), Loveland et al. “Coordinate functions of multiple complement regulating molecules, CD46, CD55 and CD59” Transpl. Proc. 26:1070 (1994), and Davies, A. and Lachmann, P. J. “Membrane defense against complement lysis: the structure and biological properties of CD59” Immunol. Res. 12: 258 (1993) related to CD59. Accordingly, we considered whether there was differential expression of either, or both, of these molecules on the cell lines tested above. Indeed, all of the cells, except the CEM line and the U937 line, expressed both of the molecules. And, indeed, the endothelial cell line ECV-304 expressed all three, CD46, CD55, and CD59. FIGS. 19 and 20, respectively. In contrast, the CEM line expressed only CD59 and the U937 line expressed only CD55. See FIG. 14. This data is useful in connection with the prediction of cells that could be selectively eradicated by ABX-CBL and consequently targeted in connection with anti-CD147 in accordance with the present invention.

Discussion of Function of ABX-CBL/CD147 Based Therapy

From the foregoing, it is clear that CBL1 and ABX-CBL operates to kill cells through the activation of complement. The combination of ABX-CBL and complement only kill activated T-cells (both CD4⁺ and CD8⁺), activated B-cells, and monocytes, but does not effect resting T-cells and B-cells because such cells do not appear to express CD147 at the same level as the activated cells. It is important, to note that monocytes are also killed by ABX-CBL and complement. This data provides an explanation for the operation of ABX-CBL therapy in diseases, such as GVHD, because, ABX-CBL selectively depletes those effector cells (activated T- and B-cells) and the antigen presenting cells (monocytes and B-cells) which ordinarily would lead to further T-cell activation.

The mode of operation of the ABX-CBL antibody, and future therapeutic molecules directed against CD147, in this regard appears to be at least partially related to, or dependent upon, each of the above-discussed functional characteristics: (i) complement mediated lysis, (ii) cellular activation, (iii) expression levels of CD147 and/or density of CD147 on the cell surface, and (iv) the absence of expression of one or more of the complement inhibitory molecules on the cell surface. Accordingly, through use of this information, it is possible to design functional assays for the prediction of efficacy of a CD147 based therapeutic.

Indeed, the desirability of mimicking ABX-CBL binding and efficacy is highlighted based upon a preliminary tissue distribution study of the ABX-CBL antibody. In the study, ABX-CBL is widely distributed throughout a variety of tissues. However, the majority of the distribution is likely to be due to nonspecific binding. Nevertheless, there appears to be specific binding in endothelial cells (venules, arterioles, but not capillary beds), smooth muscle, and some mesothelium. Also, the lymphoreticular tissues appear to be bound, although, the staining seems to be restricted to large lymphocytes, presumably activated blasts. From the study conducted, it was difficult to distinguish intracellular from extracellular staining. A certain amount of cytoplasmic staining was clearly evident and could have been related to hn-RNP-k binding.

Discussion of Results; Utilization of the ABX-CBL Antibody for the Design of Therapeutics

The above in vitro work with the ABX-CBL antibody, in combination with the association of the ABX-CBL antibody with the CD147 antigen herein, provide the first evidence that antibodies to CD147 that are capable of complement mediated killing could provide an efficacious approach to the treatment of disease. Moreover, because of CD147's wide distribution and expression in the body and the tissue binding information that indicates that the CBL1 and ABX-CBL antibody associates with many tissues, the excellent prior clinical experience with the CBL1 antibody was difficult to reconcile unless CBL1 and ABX-CBL are, for example, specific to forms of CD147 expressed on certain cells or that other factors associated with complement mediated cell killing limit the CBL1 and ABX-CBL antibody's effects to particular tissues or perhaps a combination thereof.

Criteria for Generation of CD]47 Based Therapeutics

From the foregoing, it is clear that the ABX-CBL antibody provides a powerful tool for the development of other CD147 based therapeutics. First, because of the extreme safety demonstrated to date with the CBL1 and ABX-CBL antibody, it is desirable to mimic the binding of the ABX-CBL antibody as closely as possible. Second, because of the apparent efficacy of the CBL1 antibody it is desirable, at least initially, that any new therapeutic mediate complement fixation and lysis. Accordingly, in connection with the design of other CD147 based therapeutics, it is expected that through simulating the binding (or structural aspects) and mode of operation (or functional aspects) of ABX-CBL in the therapeutic candidates, safety and efficacy can be expected.

Structural Considerations

In connection with simulating or mimicking the structural aspects of ABX-CBL binding, we expect to be able readily generate antibodies that bind to CD147 in a similar manner as ABX-CBL. With the information discussed above, we know at least three levels of detail related to ABX-CBL's binding to CD147: (i) ABX-CBL appears to bind, if not preferentially, to a form of CD147 expressed on the population of cells selected from the group consisting of activated T-cells, activated B-cells, and monocytes, (ii) ABX-CBL shows clear and specific binding to 62 KD and 35 KD molecular species on Western blot analysis, and (iii) ABX-CBL appears very specific to an epitope on CD147 (and potentially a similar epitope on hn-RNP-k protein) defined by the consensus sequence RXRSH. In addition, ABX-CBL can be utilized to “structurally” compare, screen, or act as a functional assay for additional antibody candidates to CD147 through competition studies.

As will be appreciated, the above information provides highly useful information to the generation of additional antibody candidates. Put another way, antibody candidates that are generated that possess one or more of the above-characteristics are more likely to possess similar activity to the ABX-CBL antibody. An antibody candidate that possesses greater numbers of similar characteristics is likely to be a very close mimic to the ABX-CBL antibody and, accordingly, would likely exhibit similar safety and efficacy data as the ABX-CBL antibody.

In addition, as was discussed above, we expect to be able to generate additional information related to the binding of the ABX-CBL antibody to CD147 through certain experiments designed to elucidate ABX-CBL binding, for example, through:
Additional mapping experiments related to the binding of CD147 to the ABX-CBL antibody. One such set of experiments relate to depletion experiments in which the ABX-CBL antibody bound to CD147 is cleaved with protease and the resulting products scanned with mass spectroscopy and the process repeated as necessary. Another such set of experiments relate to the isolation, purification, and understanding of the 35 KD species recognized by the ABX-CBL antibody. One method of accomplishing this is though the classical purification of the 35 KD molecule as discussed above in connection with the 62 KD species (hn-RNP-k protein). Another approach is the immunoaffinity purification of the 35 KD band through the generation of, for example, Fab fragments of the ABX-CBL antibody and binding the same to a column as discussed above in connection with the immunoaffinity purification conducted with the 2.6.1 antibody.
Experiments directed to understanding CD147 cellular development. For example, the development of CD147 on the cell surface can be gleaned through conducting “pulse-chase” experiments. In such experiments, cells (such as CEM cells) growing in culture (Met⁽⁻⁾ media) are “pulsed” with S³⁵-Met for a sufficient time periods (and varied time periods) for the label to be enrolled into the cellular protein synthesis. Thereafter, cells are washed with “cold” medium and CD147 on the cell surface can be immunoprecipitated and subjected to autoradiography. Information can be gained related to potential alternative splicings, glycosylation levels, and other developmental differences of the expressed CD147 molecules.

Experiments related to the role of glycosylation levels to ABX-CBL binding to CD147 can also be queried through reaction of CD147 with various glycosidases (see e.g., Mizukami et al. J. Immunol. 147:1331-1337 (1991), Schlosshauer Development 113:129-140 (1991), Fadool and Linser J. Neurochemistry 60:1354-1364 (1993)) and considering ABX-CBL binding to the various forms.

Functional Considerations

Once, or, concurrently with determining whether, one is satisfied with the “structure” of an antibody candidate (i.e., in connection with the antibody's binding to CD147), in accordance with the present invention, we have provided detailed functional criteria that appear important to the ABX-CBL antibody's in vivo efficacy that can be utilized to determine whether an antibody candidate is likely to operate in a similar manner to the ABX-CBL antibody. Such features include (i) cell killing through CDC, (ii) apparent effect of density or expression of the CD147 molecule on cellular populations, and (iii) the role of protective factors (for example, CD46, CD55, and CD59) on cellular populations.

As will be appreciated, the above information provides highly useful information to the generation of additional antibody candidates. Put another way, antibody candidates that are generated that possess one or more of the above-characteristics are more likely to possess similar activity to the ABX-CBL antibody. An antibody candidate that possesses greater numbers of similar characteristics is likely to be a very close mimic to the ABX-CBL antibody and, accordingly, would likely exhibit similar safety and efficacy data as the ABX-CBL antibody.

In Vivo Models

Each of the foregoing features, whether structural or functional, can essentially be carried out in vitro. Of course, however, prior to proceeding into man with therapeutic candidates it is desirable to generate in vivo data to ensure that operation of the antibody candidate will be safe and efficacious in vivo. In connection with GVHD, there are several animal models that have been shown to be highly predictive of the operation of therapeutic candidates in man. Such models include:
Murine model (Hakim F T & Mackall C L “The Immune System: Effector and Target of Graft-Versus-Host Disease” in Graft-vs. Host Disease (Ferrara et al. eds, 2d edition, Marcel Dekker, Inc., NY (1997)).
Canine Model (Storb et al. Blood 89:3048-3054 (1997); Yu et al. Bone Marrow Transplantation 17:649-653 (1996), Raff et al. Transplantation 54:813-820 (1992); and Deeg et al. Transplantation 37:62-65 (1984))
Primate Skin Graft Model (Chatterjee et al. Hybridoma 1:369-377 (1982) and Billing R. and Chatterjee S. Transplantation Proceedings 15:649-650 (1983))

As will be appreciated, in order such models to predictive, it is necessary that the antibody candidate is reactive with the endogenous form of CD147 in the animal.

Construction of Antibodies

An excellent model in which to generate therapeutic molecules targeting CD147 is in connection with the generation of antibodies. Antibodies can be generated with relative ease and are also capable of ready screening. In recent years, it has become possible to generate different “types” of antibodies; from conventional murine antibodies through human antibodies generated from transgenic animals. Within that spectrum, antibodies can also be generated through display techniques (i.e, phage), murine or other antibodies can be humanized, and the like. Some of these techniques are discussed below.

In connection with the generation of antibodies through immunization techniques, both classical and advanced immunization techniques can be used. By classical, we mean that animals can simply be immunized with the antigen, lymphocytic cells fused with myeloma cells, and hybridomas screened therefrom. By advanced, we mean that either immunization schemes can be biased or, instead of simply forming hybridomas, lymphocytic cells can be used directly to form display libraries and screened using, for example, phage or other display technologies. Such techniques are conventional in the art and are discussed in additional detail below. In connection with biasing immunizations, one can immunize with CD147, followed by immunization with peptides, such as the 15-mer peptide mentioned above. In this manner, there is a higher probability of generating antibodies that possess specificity and affinity for selected epitopes for example. Thus, it is expected that antibodies having specificity for the RXRSH consensus sequence in CD147, as discussed above, can be more readily generated. It will be appreciated that such immunization techniques can be utilized in connection with standard fusions and screening procedures or advanced screening procedures. Another set of advanced immunization techniques are related to techniques of antigen presentation (i.e., DEC systems) and techniques to augment the immune response (i.e., CD140 systems) in the animal in which the immunization is being undertaken.

Generation of Human Antibodies from Transgenic Animals

The generation of fully human antibodies, for example, from transgenic animals, is very attractive. Fully human antibodies are expected to minimize the immunogenic and allergic responses intrinsic to mouse or mouse-derived Mabs and thus to increase the efficacy and safety of the administered antibodies. The use of fully human antibodies can be expected to provide a substantial advantage in the treatment of chronic and recurring human diseases, such as inflammation, autoimmunity, and cancer, which often require repeated antibody administrations.

One approach that has been utilized in connection with the generation of human antibodies is the construction of mouse strains that are deficient in mouse antibody production but that possess large fragments of the human Ig loci so that such mice would produce a large repertoire of human antibodies in the absence of mouse antibodies. Large human Ig fragments preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains yields high affinity antibodies against any antigen of interest, including human antigens. Using hybridoma technology, antigen-specific human Mabs with the desired specificity can be readily produced and selected.

This general strategy was demonstrated in connection with the generation of the first XenoMouse strains as published in 1994. See Green et al. Nature Genetics 7:13-21 (1994). The XenoMouse strains were engineered with 245 kb and 190 kb-sized germline configuration fragments of the human heavy chain loci and kappa light chain loci, respectively, which contained core variable and constant region sequences. Id The human Ig containing yeast artificial chromosomes (YACs) proved to be compatible with the mouse system for both rearrangement and expression of antibodies, and were capable of substituting for the inactivated mouse Ig genes. This was demonstrated by their ability to induce B-cell development and to produce an adult-like human repertoire of fully human antibodies and to generate antigen-specific human Mabs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization.

Such approach is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996. See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, and PCT Application No. PCT/US96/05928, filed Apr. 29, 1996. The disclosures of each of the above-cited patents and applications are hereby incorporated by reference in their entirety.

In an alternative approach, others, including GenPharm International, Inc., have utilized a “minilocus” strategy. In the minilocus strategy, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al., U.S. Pat. Nos. 5,545,806, 5,625,825, 5,661,016, 5,633,425, and 5,625,126, each to Lonberg and Kay, U.S. Pat. No. 5,643,763 to Dunn and Choi, U.S. Pat. No. 5,612,205 to Kay et al., U.S. Pat. No. 5,591,669 to Krimpenfort and Berns, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, Ser. No. 08/544,404, filed Oct. 10, 1995, the disclosures of which are hereby incorporated by reference. See also International Patent Application Nos. WO 97/13852, published Apr. 17, 1997, WO 94/25585, published Nov. 10, 1994, WO 93/12227, published Jun. 24, 1993, WO 92/22645, published Dec. 23, 1992, WO 92/03918, published Mar. 19, 1992, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), the disclosures of which are hereby incorporated by reference in their entirety.

The inventors of Surani et al., cited above, and assigned to the Medical Research Counsel (the “MRC”), produced a transgenic mouse possessing an Ig locus through use of the minilocus approach. The inventors on the GenPharm International work, cited above, Lonberg and Kay, following the lead of the present inventors, proposed inactivation of the endogenous mouse Ig locus coupled with substantial duplication of the Surani et al. work.

An advantage of the minilocus approach is the rapidity with which constructs including portions of the Ig locus can be generated and introduced into animals. Commensurately, however, a significant disadvantage of the minilocus approach is that, in theory, insufficient diversity is introduced through the inclusion of small numbers of V, D, and J genes. Indeed, the published work appears to support this concern. B-cell development and antibody production of animals produced through use of the minilocus approach appear stunted. Therefore, the present inventors have consistently urged introduction of large portions of the Ig locus in order to achieve greater diversity and in an effort to reconstitute the immune repertoire of the animals.

It will be appreciated that through use of the above-technology, human antibodies can be generated to, for example, CD147 expressing cells, CD147 itself, forms of CD147, epitopes or peptides thereof, and expression libraries thereto (see e.g. U.S. Pat. No. 5,703,057) through immunization of a transgenic mouse therewith, forming hybridomas, and screening the resulting hybridomas as described above for the activities described above.

Indeed, through use of the above-discussed technology, we prepared a panel of human monoclonal antibodies that bind CD147 through immunization of XenoMouse™ strains of transgenic mice (see Mendez et al., (1997), supra. and U.S. patent application Ser. No. 08/759,620, filed Dec. 3, 1996), available from Abgenix, Inc., Fremont, Calif. Such antibodies were further screened for their ability to compete with ABX-CBL for binding with CD147. In such panel, both human IgG2 and human IgM antibodies were detected that bound to CD147 and were capable of competition with ABX-CBL for binding to CD147. The hybridomas expressing such antibodies were designated as follows:

IgMs: CEM 10.1 C3, CEM 10.1 G10, CEM 10.12 F3, CEM 10.12 G5 CEM 13.12, CEM 13.5; and

IgG2s: 2.4.4, 2.1.1, 2.3.2, 2.6.1.

Each of the above antibodies were sequenced through isolating cDNAs encoding them from the corresponding hybridomas through RT-PCR. Germline gene identifications were made and the sequences of the antibodies compared to the germline sequences. Germline gene identifications are provided in the following Table:

TABLE 1

| | Heavy/ | | | |
|---|---|---|---|---|
| Antibody | Light | V_{H} or V_{κ} | D | J_{H} or J_{κ} |
| CEM 10-1 C3 | Heavy | V4-34 | D2/D2-15 | JH6b |
| | Light | A3/A19/DPK15 | | JK1 |
| CEM 10.1 G10 | Heavy | DP71 (V4-59) | D1-26 | JH6b |
| | Light | A30 | | JK1 |
| | | | | (not |
| | | | | identical seq) |
| CEM 10.12 F3 | Heavy | DP15 (V1-8) | D1-26 | JH6b |
| | Light | B3/DPK24 | | JK1 |
| CEM 10.12 G5 | Heavy | DP15 (V1-8 | D6-19 | JH6b |
| | Light | A30 | | JK1 |
| CEM 13.12 | Heavy | V4-34 | D2-2/D4 | JH6b |
| | Light | A3/A19/DPK15 | | JK3 |
| CEM 13.5 | Heavy | DP77-WH16 | D6-19 | JH4b |
| | | (3-21) | | |
| | Light | B3/DPK24 | | JK1 (not |
| | | | | identical seq) |
| 2.4.4 | Heavy | VII-5 | D21-9/D3-22 | JH4b |
| | Light | A2 DPK12 | | JK4 |
| 2.1.1 | Heavy | DP77 | D6-19 | JH4b |
| | Light | LFVK431 | | JK3 |
| 2.3.2 | Heavy | VII-5 | D21-9/D3-22 | JH4b |
| | Light | A2 DPK12 | | JK4 |
| 2.6.1 | Heavy | DP47 | DXP4 | JH4b |
| | Light | LFVK431 | | JK3 |

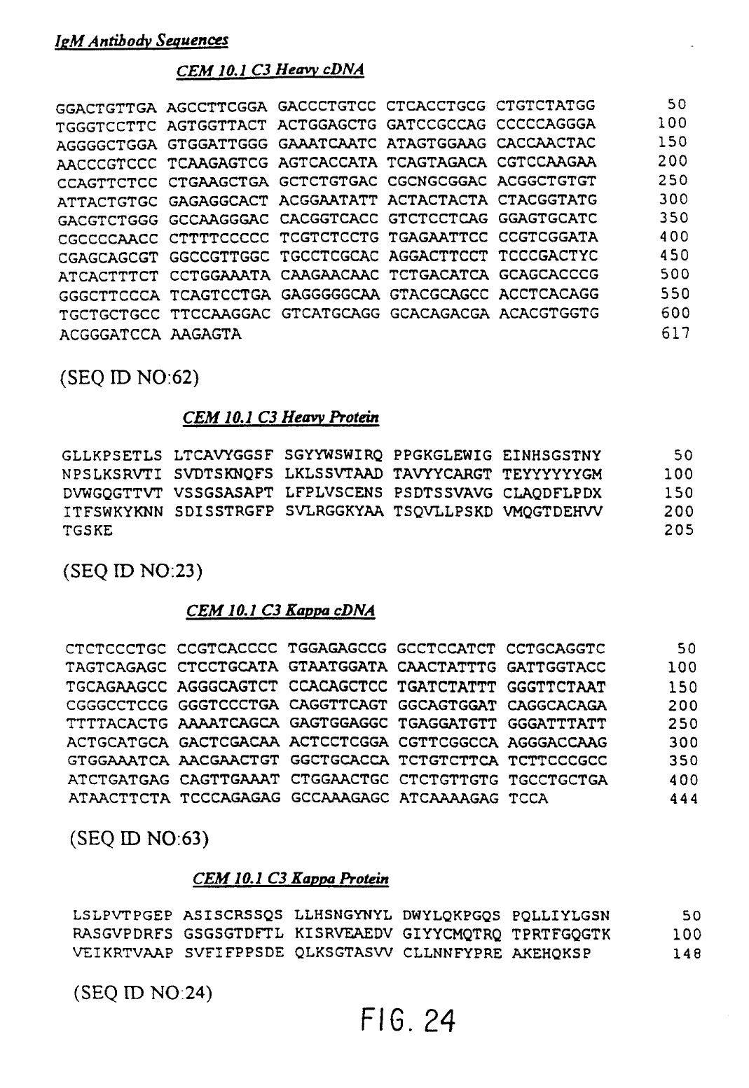

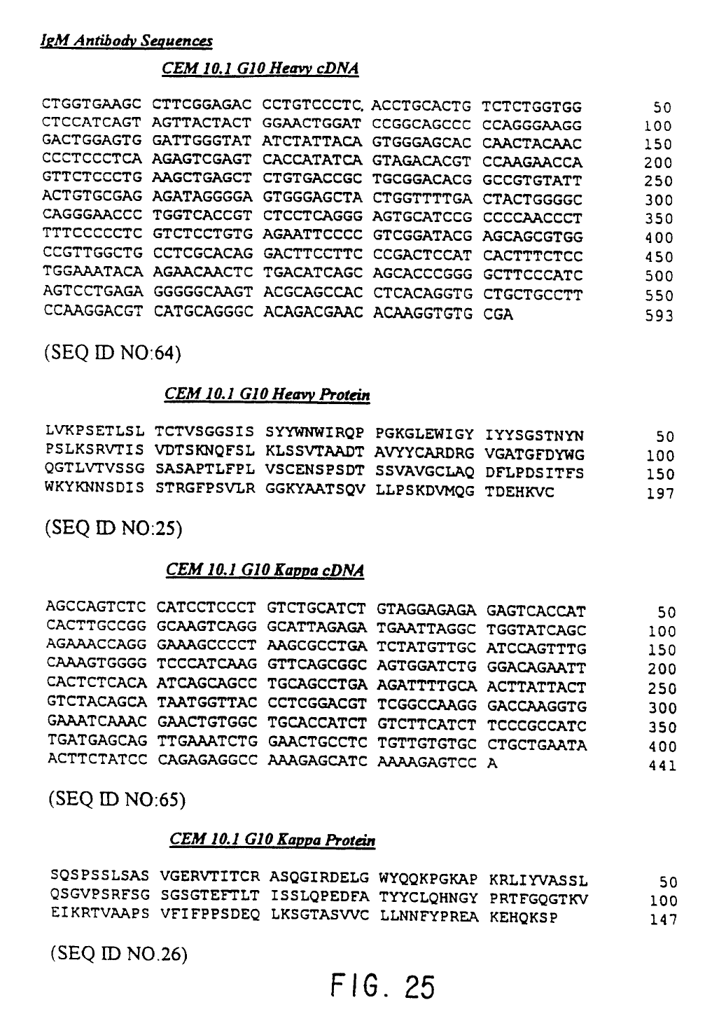

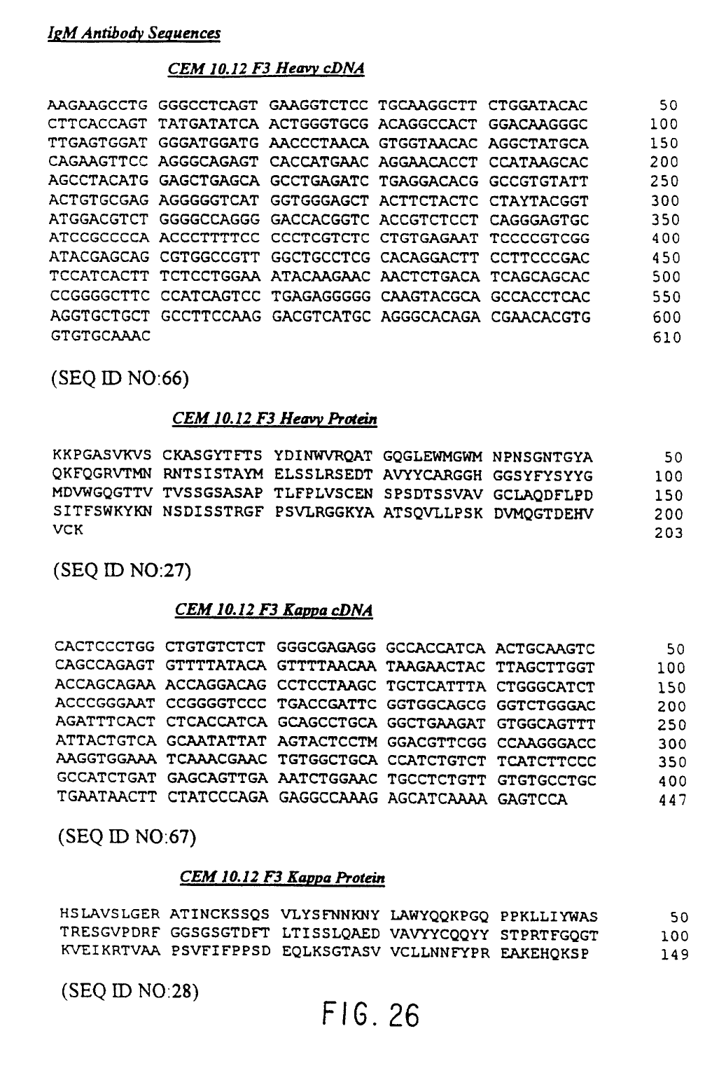

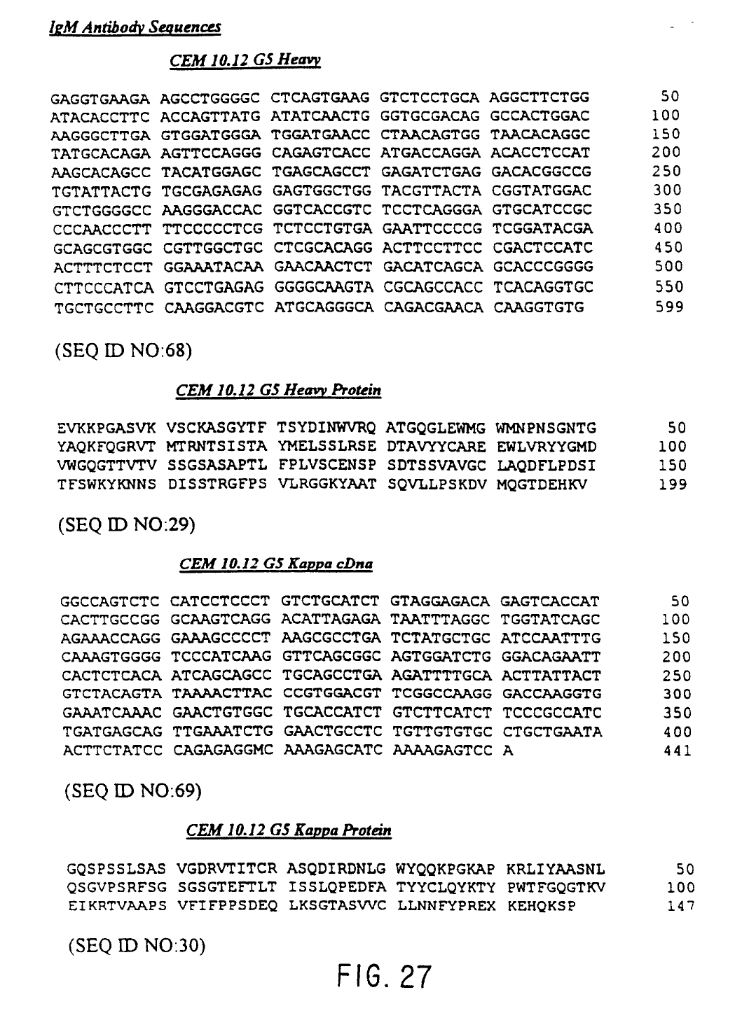

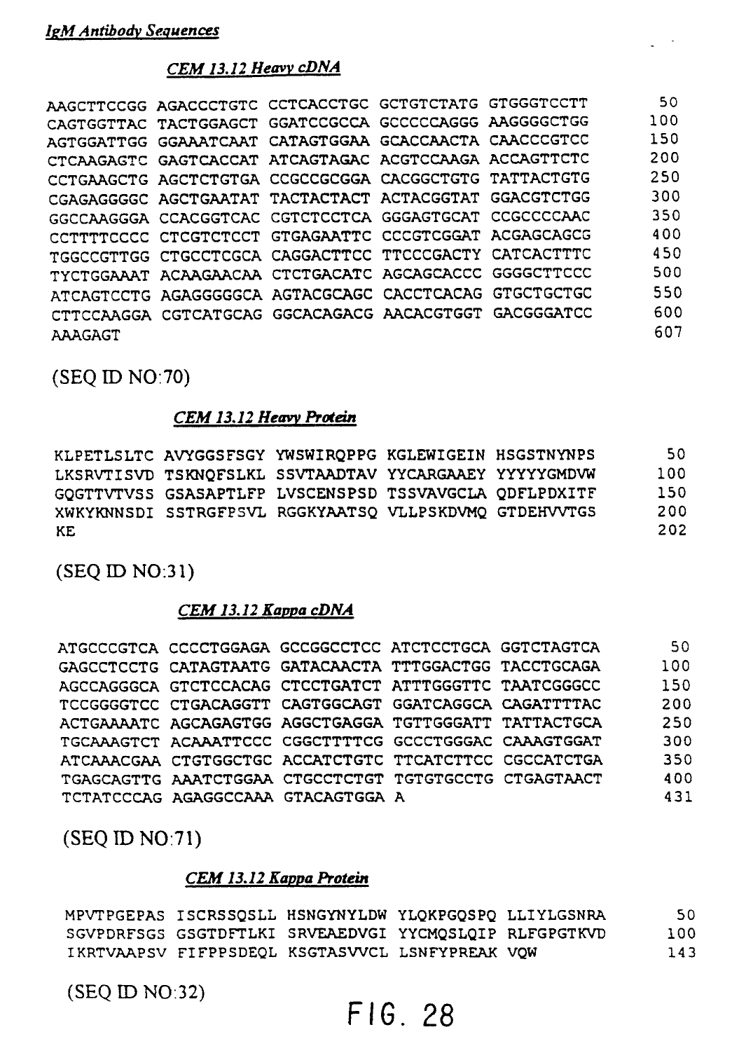

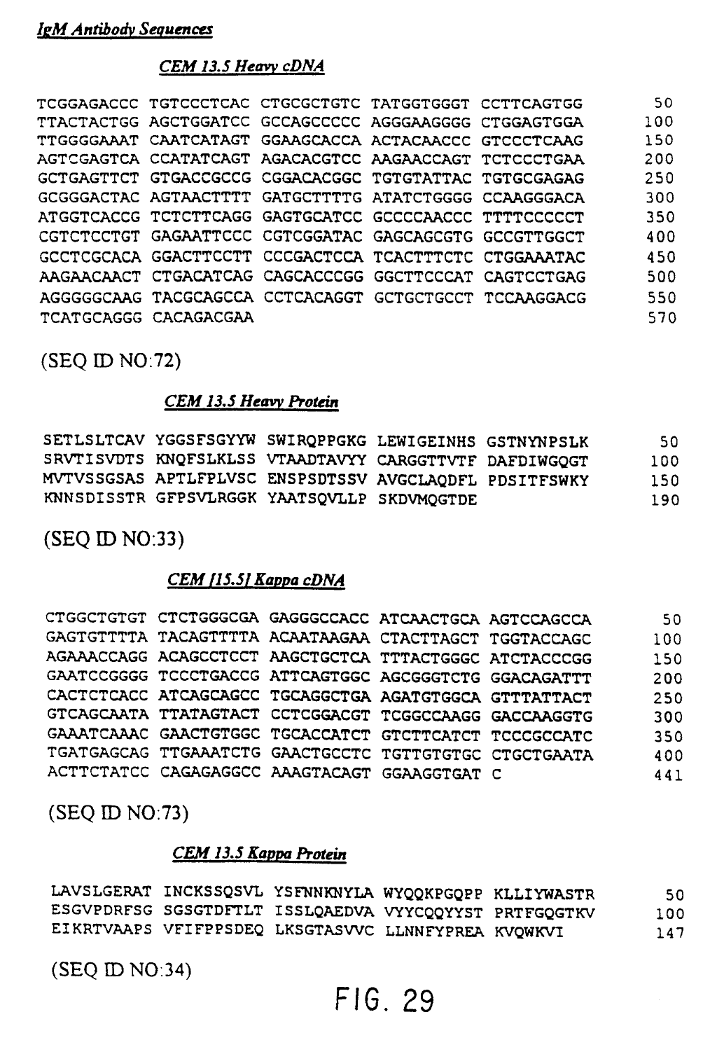

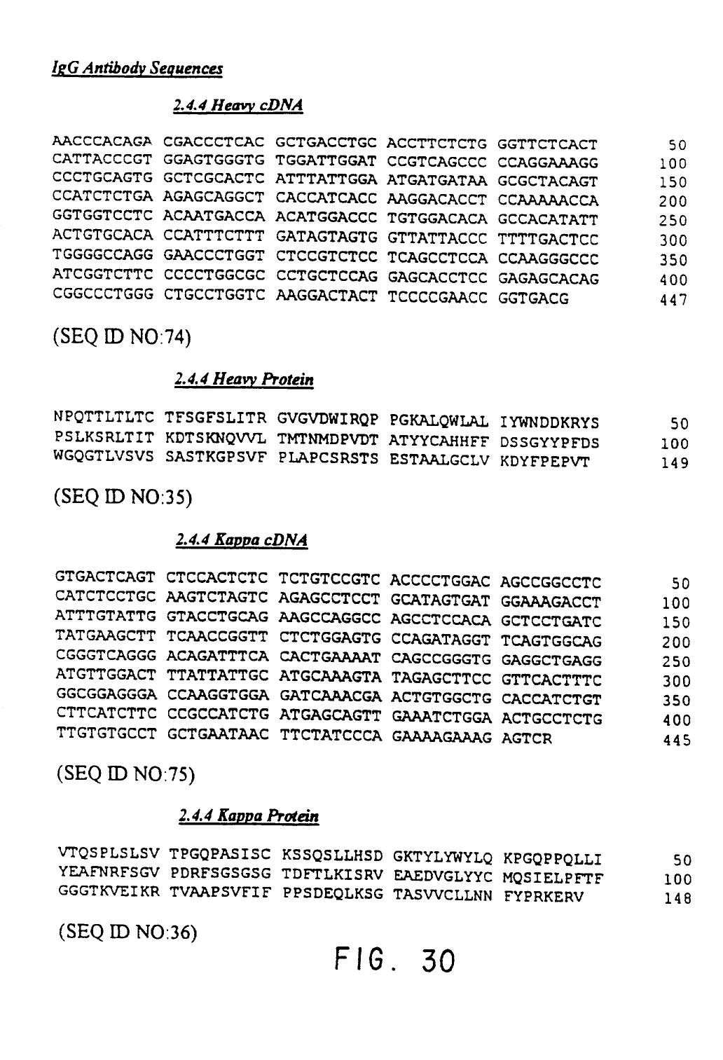

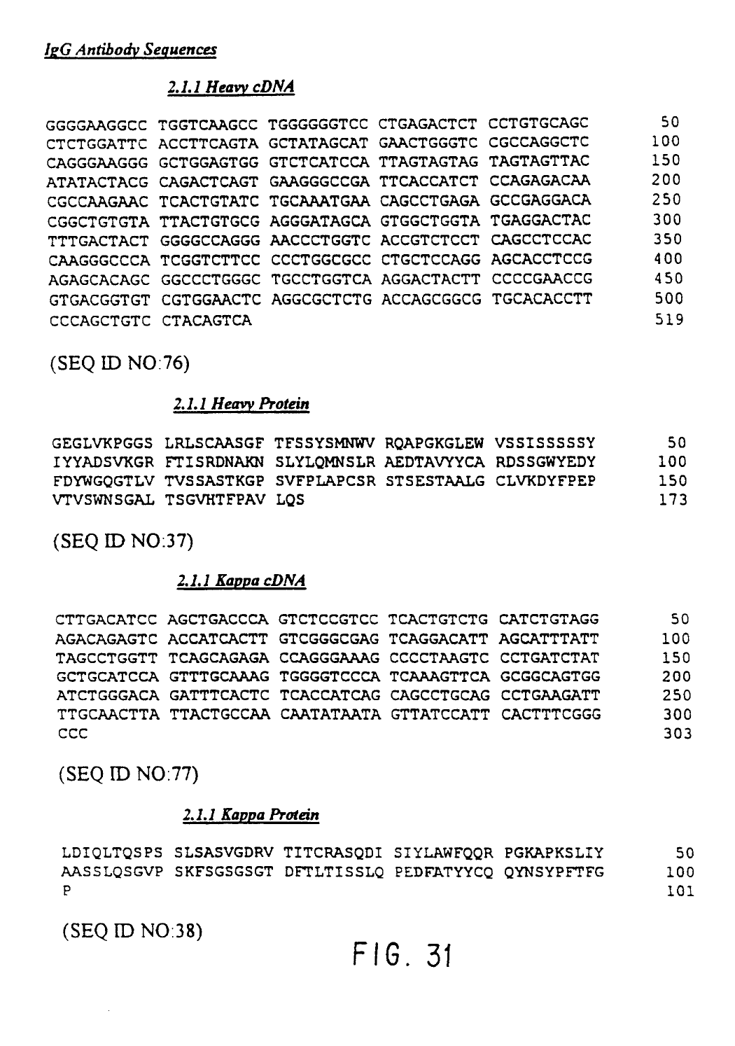

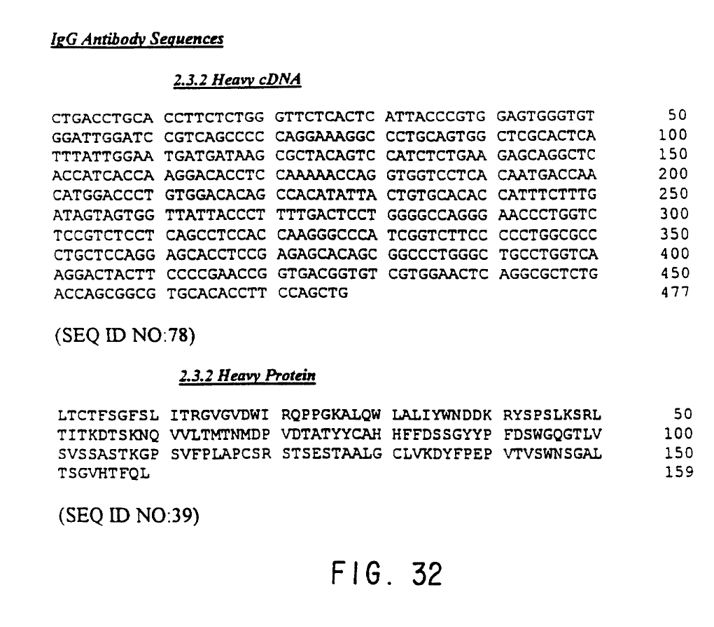

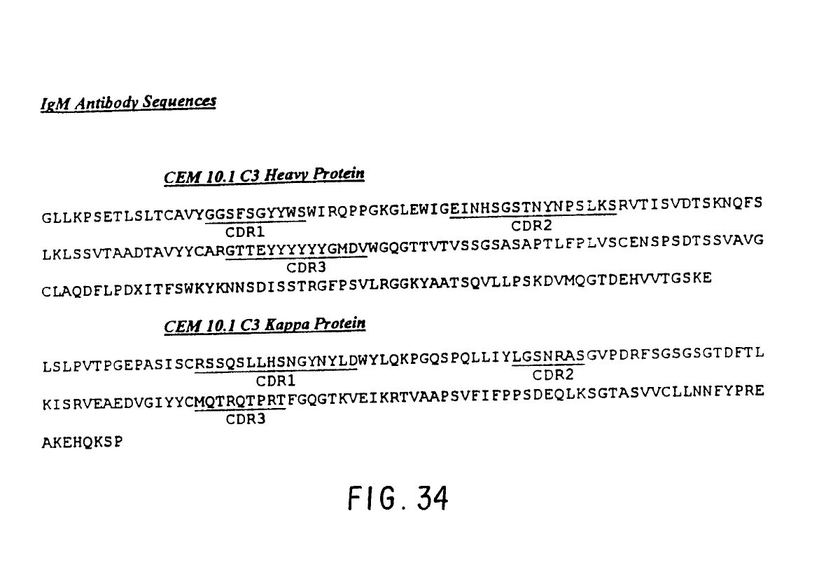

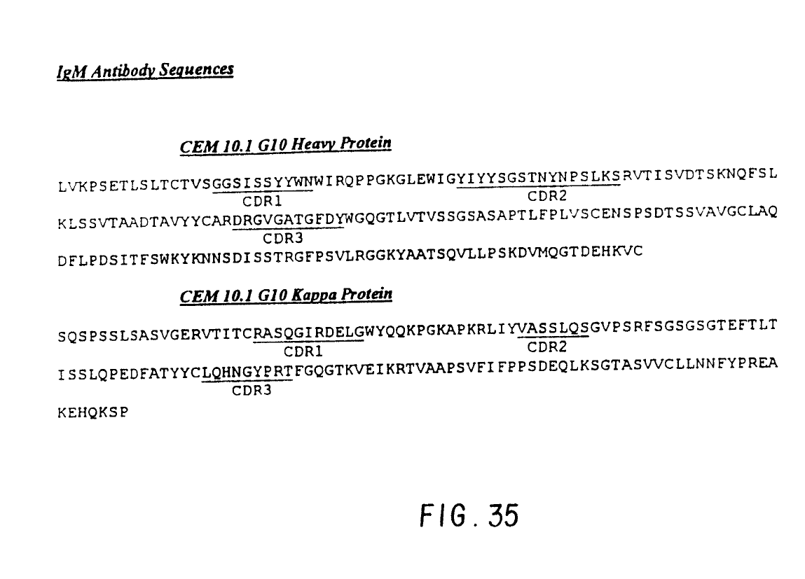

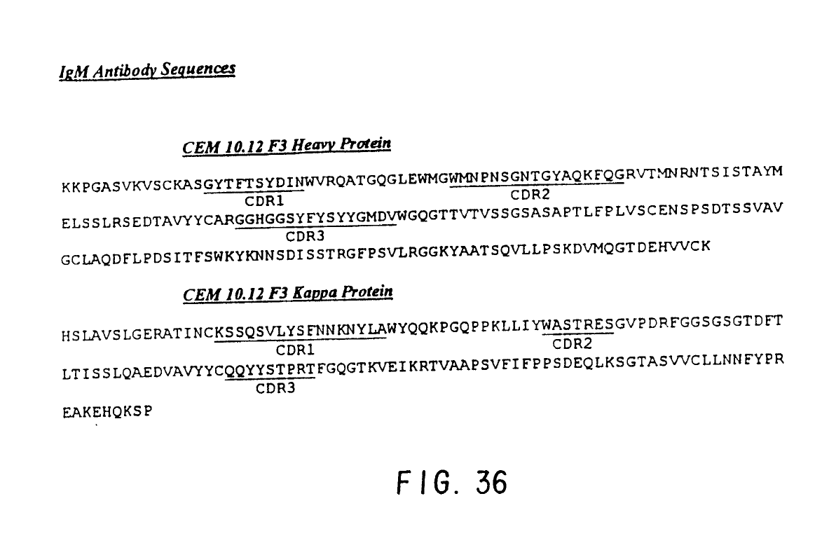

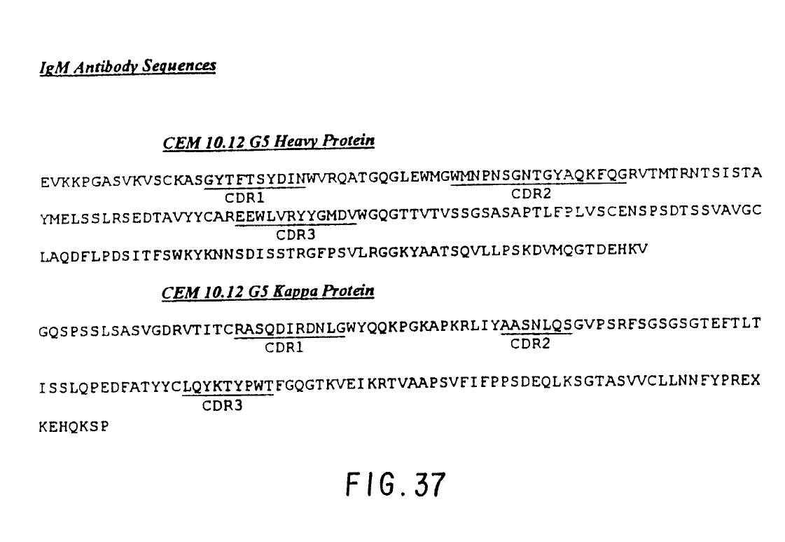

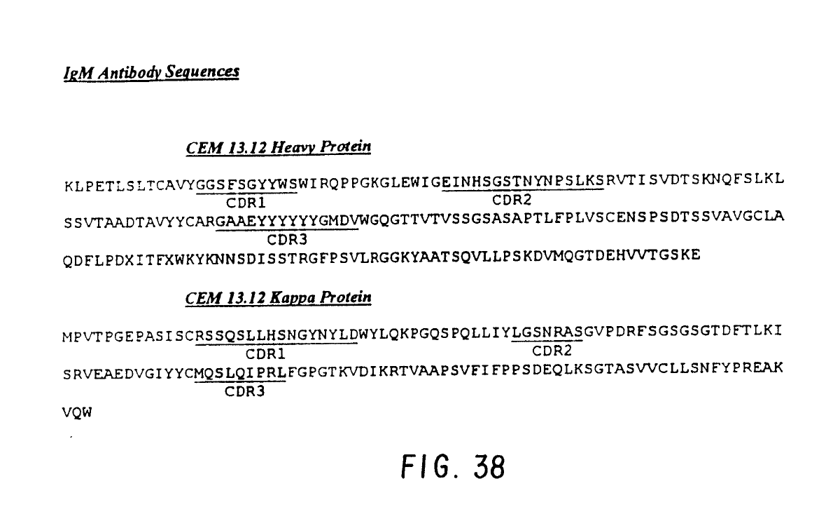

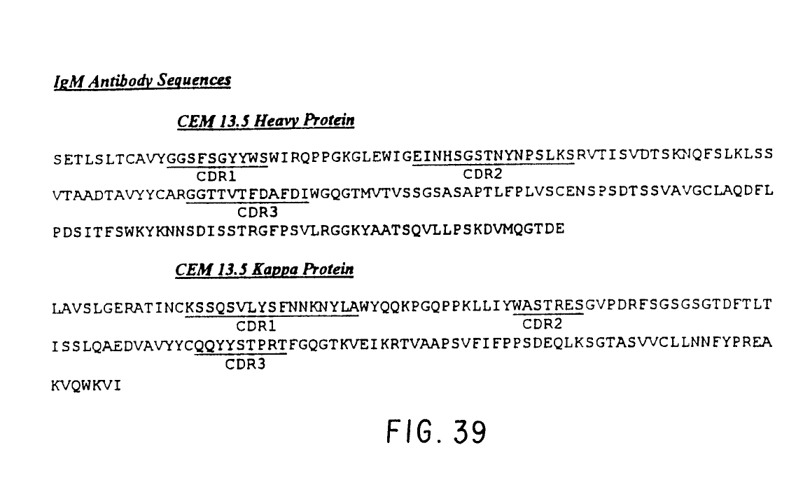

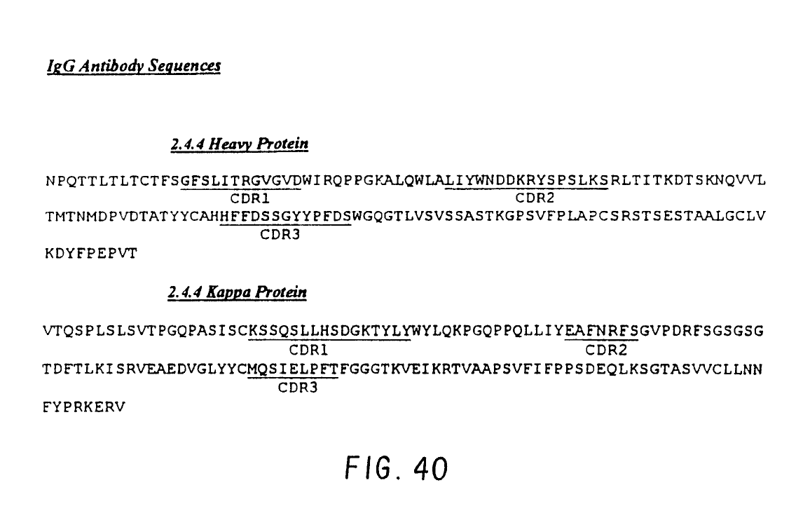

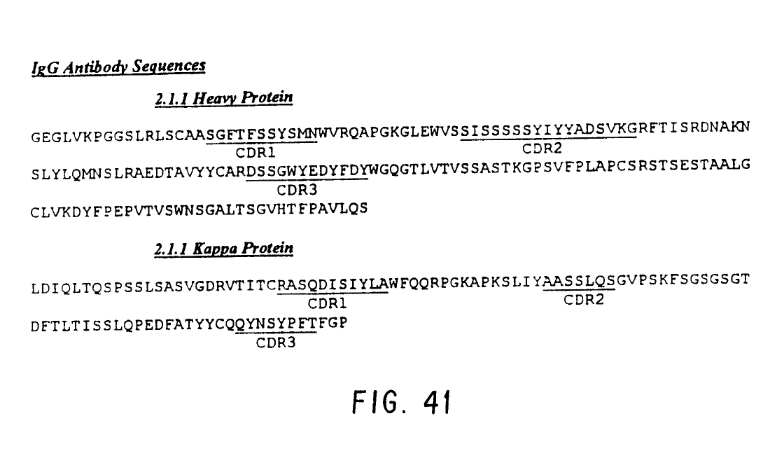

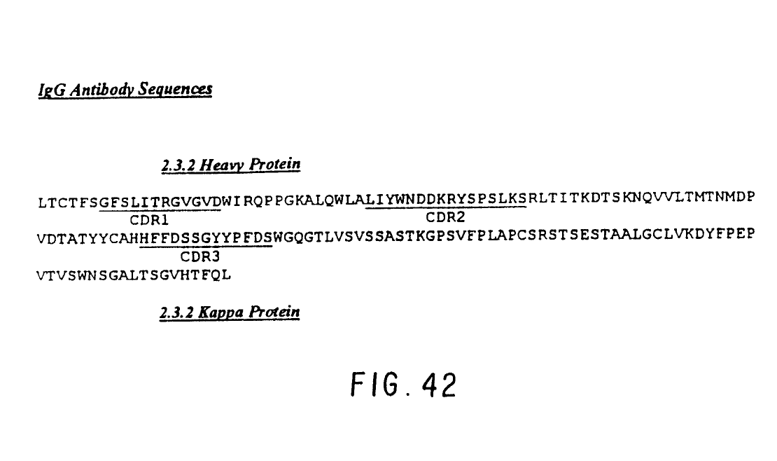

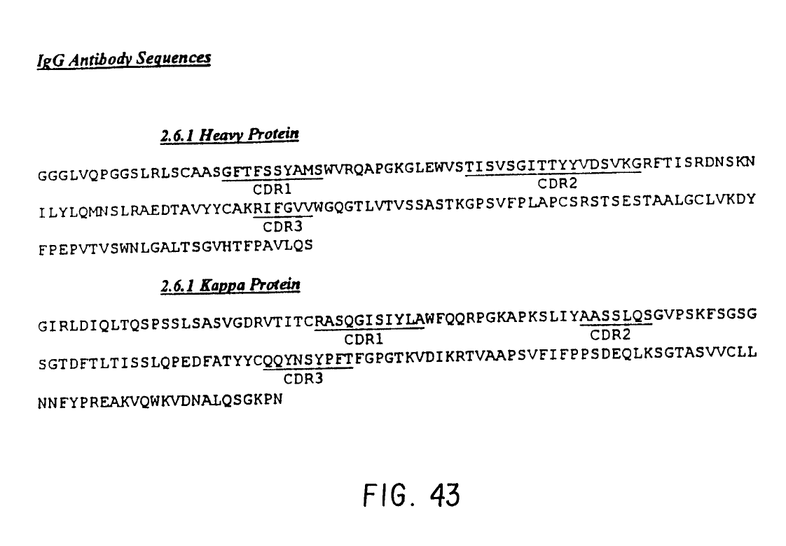

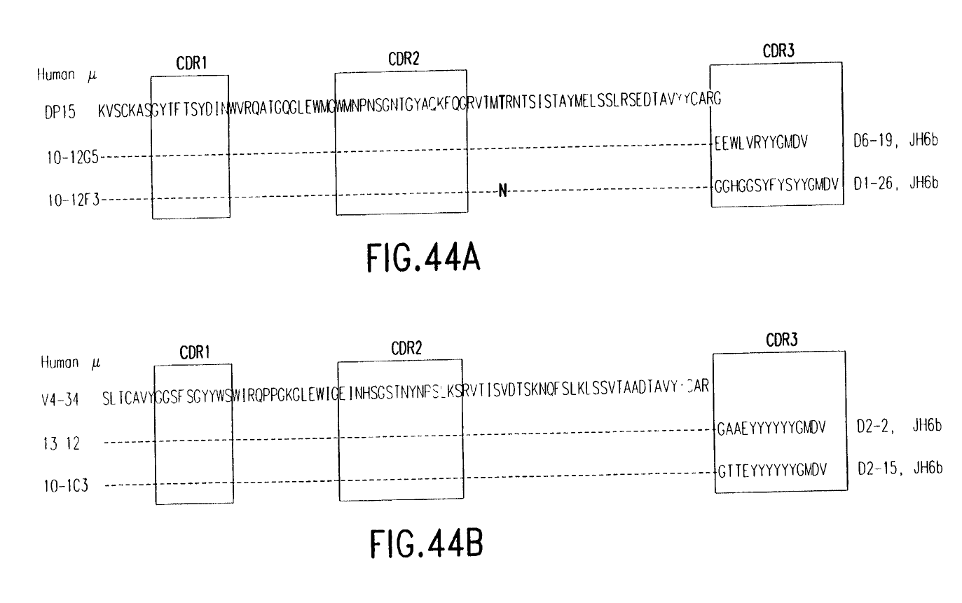

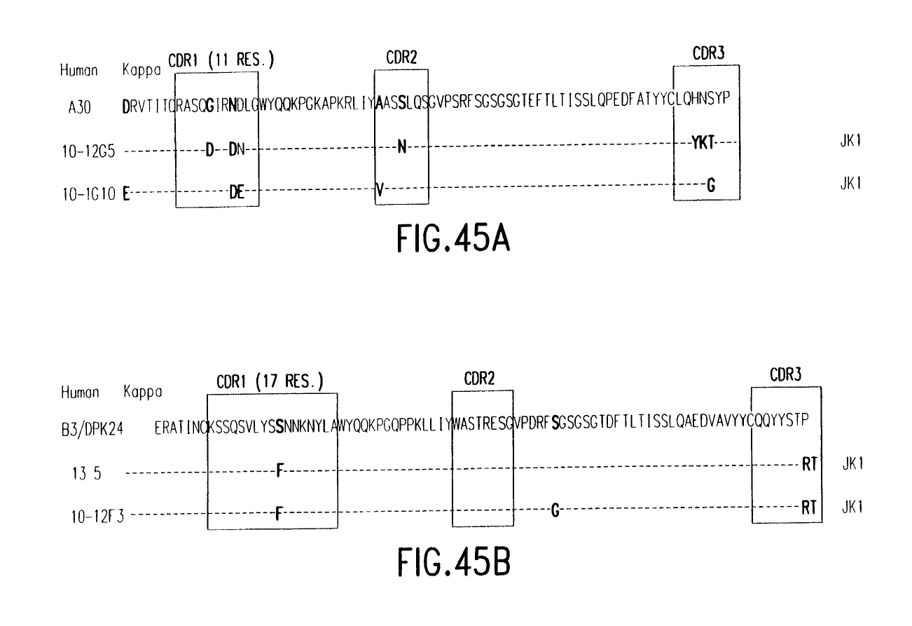

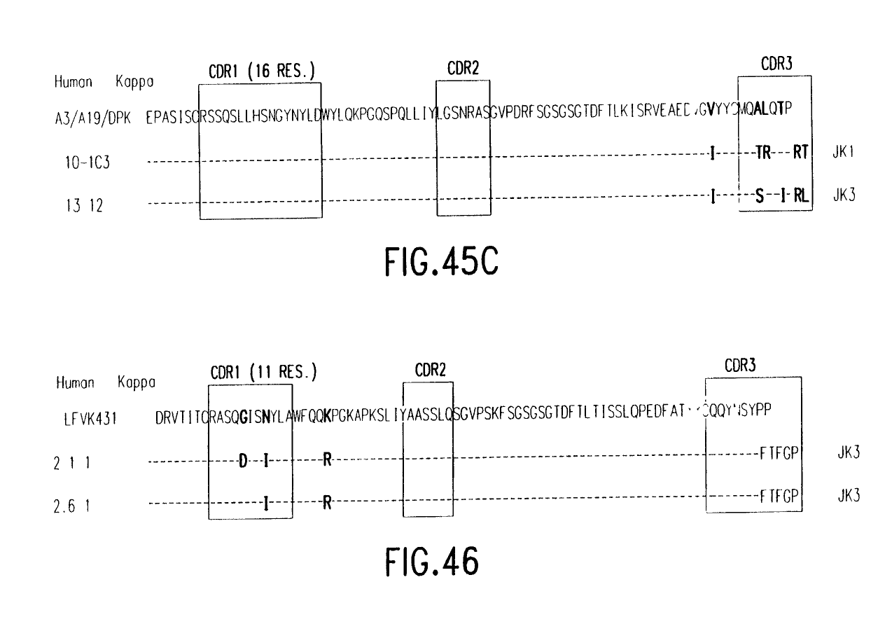

Germline sequences of the V_{H}, D, J_{H}, V_{K}, and J_{K} genes are available on GenBank The sequences of certain of the antibodies were compared to transcripts of the germline V-gene segments to observe somatic mutations in the amino acid sequences. Such sequence comparisons are shown in FIGS. 44 through 46. cDNA sequences and protein transcripts of and for each of the antibodies are shown in FIGS. 24 through 33. In addition, CDRs, according to Kabat numbering scheme, of the heavy chains and kappa light chains of the antibodies are shown in FIGS. 34 through 43.

It will be appreciated that CDRs of the above antibodies are generally very important in connection with antibody binding to an antigen. Accordingly, it will be understood that a variety of FR and other modifications can be made in and to antibodies that do not modify an antibodies binding the epitope on an antigen. Thus, an important factor in an antibody's activity is the epitope on an antigen to which an antibody binds. So long as the epitope binding is conserved, in many ways it may matter little if the primary sequence of the antibody is modified. Therefore, where sequences are discussed herein, it is submitted that the sequence of an antibody may initially define an efficacious epitope on the antigen, however, once the epitope is identified by the antigen, any antibody that binds to the same epitope on the is contemplated herein.

In view of a number of tests that were conducted, the 2.6.1 IgM antibody was chosen for additional development. As will be appreciated, all of the IgMs that were generated were monovalent. Accordingly, in order to prepare a fully human multimeric IgM antibody, we cloned the human J-chain gene from human buffy coat cells, prepared a first expression vector containing the 2.6.1 kappa light chain cDNA and the J-chain cDNA and a second expression vector containing the 2.6.1 heavy chain cDNA, cotransfected DHFR⁻ Chinese hamster ovary cells with the two vectors, and selected clones expressing the multimeric IgM.

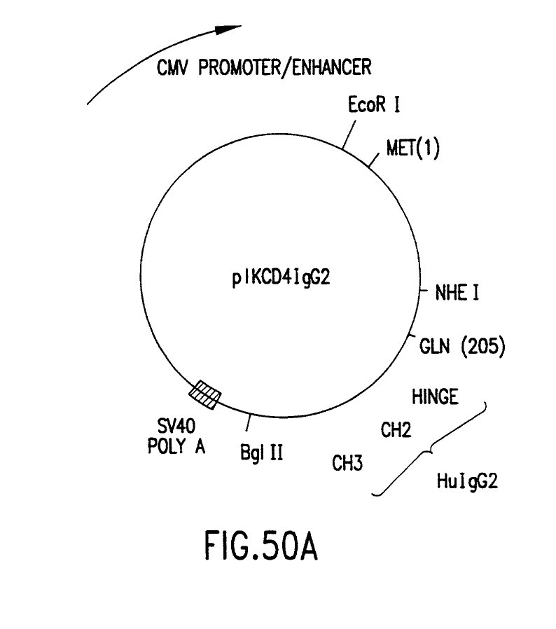

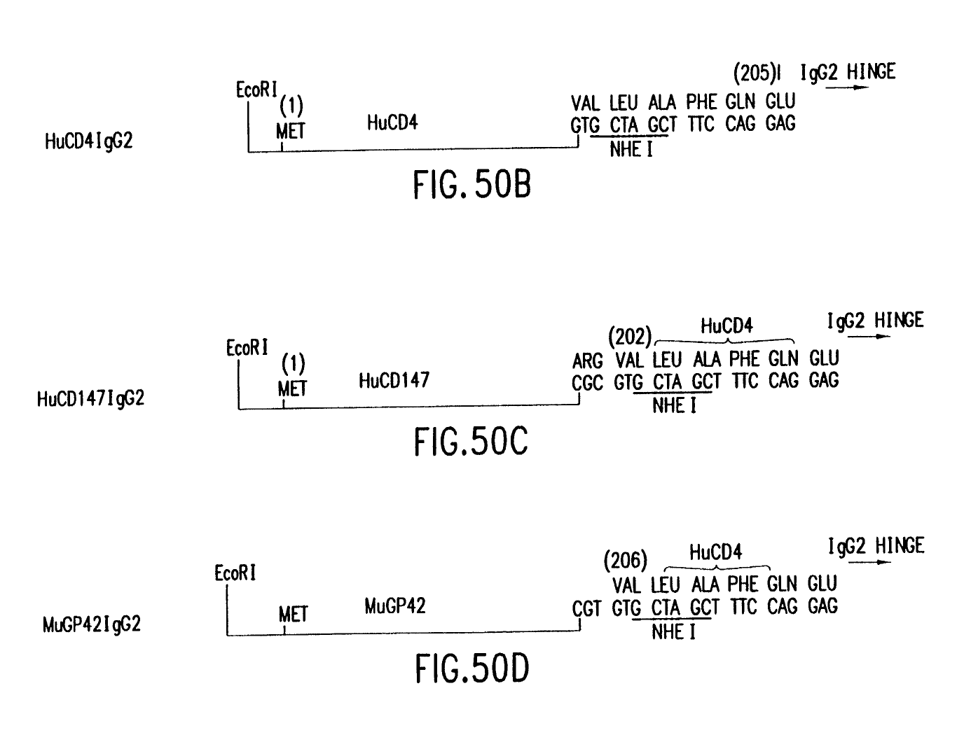

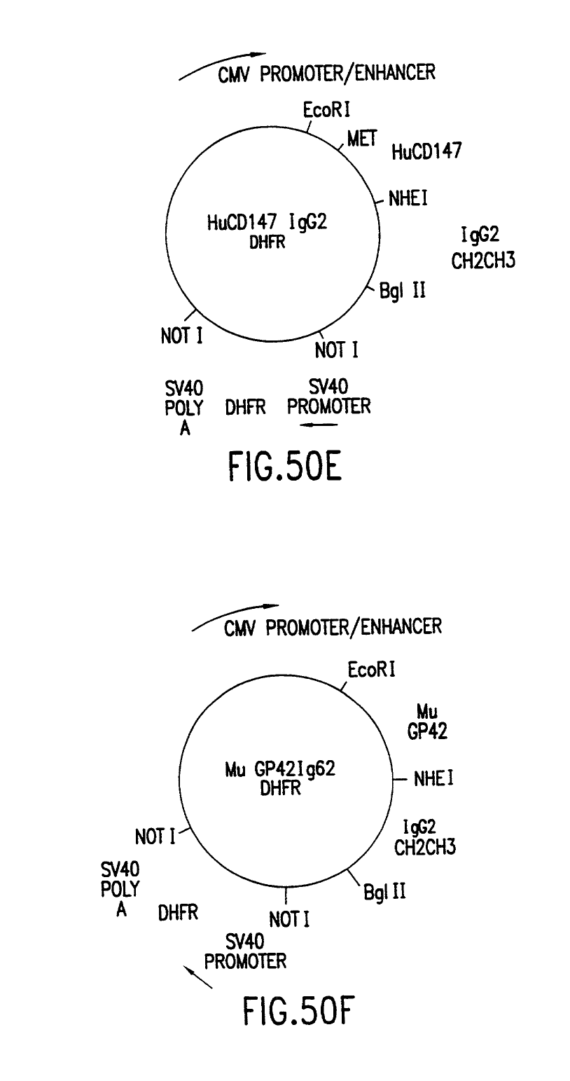

The 2.6-1 IgM+J-Chain antibody was capable of acting in ADCC as shown in FIG. 50.

Humanization and Display Technologies

As was discussed above in connection with human antibody generation, there are advantages to producing antibodies with reduced immunogenicity. To a degree, this can be accomplished in connection with techniques of humanization and display techniques using appropriate libraries. It will be appreciated that murine antibodies or antibodies from other species can be humanized or primatized using techniques well known in the art. See e.g., Winter and Harris Immunol Today 14:43-46 (1993) and Wright et al. Crit, Reviews in Immunol. 12125-168 (1992). Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau PNAS USA 94:4937-4942 (1997) (ribosomal display), Parmley and Smith Gene 73:305-318 (1988) (phage display), Scott TIBS 17:241-245 (1992), Cwirla et al. PNAS USA 87:6378-6382 (1990), Russel et al. Nucl. Acids Research 21:1081-1085 (1993), Hoganboom et al. Immunol. Reviews 130:43-68 (1992), and Chiswell and McCafferty TIBTECH 10:80-84 (1992). If display technologies are utilized to produce antibodies that are not human, such antibodies can be humanized as described above.

Using these techniques, antibodies can be generated to CD147 expressing cells, CD147 itself, forms of CD147, epitopes or peptides thereof, and expression libraries thereto (see e.g. U.S. Pat. No. 5,703,057) which can thereafter be screened as described above for the activities described above.

Further, the sequence for the active antibody from the deposited hybridoma cell line expressing the ABX-CBL antibody was previously unknown. In view of our findings discussed above that the IgM antibody was the entity responsible for the activity of the CBL1 antibody and the fact that neither the presence nor the absence of the MOPC21 light chain appeared to be advantageous nor detrimental to the activity of the antibody, we cloned the heavy chain and the kappa light chains from the IgM (ABX-CBL) producing hybridoma through RT-PCR and sequenced the cDNAs. The results of such sequencing studies, including the cDNA sequences of the heavy chain and kappa light chain and the protein transcripts thereof are shown below:

| ABX-CBL Heavy Chain Nucleotide Sequence | |
|---|---|
| (SEQ ID NO: 81) | |
| ATGTACTTGG GACTGAACTA TGTATTCATA GTTTTTCTCT TAAATGGTGT | 50 |
| CCAGAGTGAA GTGAAGCTTG AGGAGTCTGG AGGAGGCTTG GTGCAACCTG | 100 |
| GAGGATCCAT GAAACTCTCC TGTGTTGCCT CTGGATTCAC TTTCAGTAAC | 150 |
| TACTGGATGA ACTGGGTCCG CCAGTCTCCA GAGAAGGGGC TTGAGTGGGT | 200 |
| TGCTGAAATT AGATTGAANF CTAATAATTA TGCAACACAT TATGCGGAGT | 250 |
| CTGTGAAAGG GAGGTTCACC ATCTCAAGAG ATGATTCCAA AAGTAGTGTC | 300 |
| TACCTGCAAA TGAACAACTT AAGAGCTGAA GACACTGGCA TTTATTACTG | 350 |
| TACGGATTAC GATGCTTACT GGGGCCAAGG GACTCTGGTC ACTGTCTCTG | 400 |
| CAGAGAGTCA GTCCTTCCCA AATGTCTTCC CCCTCGTCTC CTGCGAGAGC | 450 |
| CCCCTGTCTG ATAAGAATCT GGTGGCCATG GGCTGCCTGG CCCGGGACTT | 500 |
| CCTGCCCAGC ACCATTTCCT TCACCTGGAA CTACCAGAAC AACACTGAAG | 550 |
| TCATCCAGGG TATCAGAACC TTCCCAACAC TGAGGACAGG GGGCAAGTAC | 600 |
| CTAGCCACCT CGCAGGTGTT GCTGTCTCCC AAGAGCATCC TTGAAGGTTC | 650 |
| AGATGAATAC CTGGTATGCA AAATCCACTA CGGAGGCAAA AACAGAGATC | 700 |
| TGCATGTGCC CATTCCAGCT GTCGCAGAGA TGAACCCCAA TGTAAATGTG | 750 |
| TTCGTCCCAC CACGGGATGG CTTCTCTGGC CCTGCACCAC GCAAGTCTAA | 800 |
| ACTCATCTGC GAGGCCACGA ACTTCACTCC AAAACCGATC ACAGTATCCT | 850 |
| GGCTAAAGGA TGGGAAGCTC GTGGAATCTG GCTTCACCAC AGATCCGGTG | 900 |
| ACCATCGAGA ACAAAGGATC CACACCCCAA ACCTACAAGG TCATAAGCAC | 950 |
| ACTTACCATC TCTGAAATCG ACTGGCTGAA CCTGAATGTG TACACCTGCC | 1000 |
| GTGTGGATCA CAGGGGTCTC ACCTTCTTGA AGAACGTGTC CTCCACATGT | 1050 |
| GCTGCCAGTC CCTCCACAGA CATCCTAACC TTCACCATCC CCCCCTCCTT | 1100 |
| TGCCGACATC TTCCTCAGCA AGTCCGCTAA CCTGACCTGT CTGGTCTCAA | 1150 |
| ACCTGGCAAC CTATGAAACC CTGAATATCT CCTGGGCTTC TCAAAGTGGT | 1200 |
| GAACCACTGG AAACCAAAAT TAAAATCATG GAAAGCCATC CCAATGGCAC | 1250 |
| CTTCAGTGCT AAGGGTGTGG CTAGTGTTTG TGTGGAAGAC TGGAATAACA | 1300 |
| GGAAGGAATT TGTGTGTACT GTGACTCACA GGGATCTGCC TTCACCACAG | 1350 |
| AAGAAATTCA TCTCAAAACC CAATGAGGTG CACAAACATC CACCTGCTGT | 1400 |
| GTACCTGCTG CCACCAGCTC GTGAGCAACT GAACCTGAGG GAGTCAGCCA | 1450 |
| CAGTCACCTG CCTGGTGAAG GGCTTCTCTC CTGCAGACAT CAGTGTGCAG | 1500 |
| TGGCTTCAGA GAGGGCAACT CTTGCCCCAA GAGAAGTATG TGACCAGTGC | 1550 |
| CCCGATGCCA GAGCCTGGGG CCCCAGGCTT CTACTTTACC CACAGCATCC | 1600 |
| TGACTGTGAC AGAGGAGGAA TGGAACTCCG GAGAGACCTA TACCTGTGTT | 1650 |
| GTAGGCCACG AGGCCCTGCC ACACCTGGTG ACCGAGAGGA CCGTGGACAA | 1700 |
| GTCCACTGGT AAACCCACAC TGTACAATGT CTCCCTGATC ATGTCTGACA | 1750 |
| CAGGCGGCAC CTGCTATTGA CCAT | 1774 |
| ABX-CBL Heavy Chain Protein Sequence | |
| (SEQ ID NO:18) | |
| EVKLEESGGG LVQPGGSMKL SCVASGFTFS NYWMNWVRQS PEKGLEWVAE | 50 |
| IRLKSNNYAT HYAESVKGRF TISRDDSKSS VYLQMNNLRA EDTGIYYCTD | 100 |
| YDAYWGQGTL VTVSAESQSF PNVFPLVSCE SPLSDKNLVA MGCLARDFLP | 150 |
| STISFTWNYQ NNTEVIQGIR TFPTLRTGGK YLATSQVLLS PKSILEGSDE | 200 |
| YLVCKIHYGG KNRDLHVPIP AVAEMNPNVN VFVPPRDGFS GPAPRKSKLI | 250 |
| CEATNFTPKP ITVSWLKDGK LVESGFTTDP VTIENKGSTP QTYKVISTLT | 300 |
| ISEIDWLNLN VYTCRVDHRG LTFLKNVSST CAASPSTDIL TFTIPPSFAD | 350 |
| IFLSKSANLT CLVSNLATYE TLNISWASQS GEPLETKIKI MESHPNGTFS | 400 |
| AKGVASVCVE DWNNRKEFVC TVTHRDLPSP QKKFISKPNE VHKHPPAVYL | 450 |
| LPPAREQLNL RESATVTCLV KGFSPADISV QWLQRGQLLP QEKYVTSAPM | 500 |
| PEPGAPGFYF THSILTVTEE EWNSGETYTC VVGHEALPHL VTERTVDKST | 550 |
| GKPTLYNVSL IMSDTGGTCY | 570 |
| ABX-CBL Light Chain Protein Sequence | |
| (SEQ ID NO:19) | |
| KFLLVSAGDR VTITCKASQS VSNDVAWYQQ KPGQSPKLLI YYASNRYTGV | 50 |
| PDRFTGSGYG TDFTFTISTV QAEDLAVYFC QQDYSSPYTF GGGTKLEIKR | 100 |
| ADAAPTVSIF PPSSEQLTSG GASVVCFLNN FYPKDINVKW KIDGSERQNG | 150 |
| VLNSWTDQDS KDSTYSMSST LTLTKDEYER HNSYTCEATH KTSTSPIVKS | 200 |
| FNRNEC | 206 |

As will be appreciated, through utilization of the sequence, it is possible to prepare a humanized version of the ABX-CBL antibody. In general, the nucleotide sequences encoding the CDRs are grafted into human framework (FR) sequences using conventional techniques. Alternatively, amino acid residues in the framework regions surrounding the CDRs (i.e., residues in FR1 and FR2, surrounding CDR1, FR2 and FR3, surrounding CDR2, and/or FR3 and FR4, surrounding CDR3) are modified through mutagenesis of cDNAs encoding the same also using conventional techniques. In either case, the modified cDNAs encoding the humanized kappa light chain and the heavy chain are generally then introduced into a cell line for expression (i.e., NSO, CHO, or the like) either directly, through cotransfection, or through use of the cell-cell fusion techniques described in U.S. patent application Ser. No. 08/730,639, filed Oct. 11, 1996 or International Patent Application No. WO 98/16654, published Apr. 23, 1998. Thereafter, the humanized antibodies are expressed and assayed for binding and other functional attributes. The molecules can be iteratively modified at the DNA level as desired or necessary to achieve improved binding or other functional attributes of the antibodies. For example, in certain cases, it is necessary to reintroduce murine sequences within the human FRs to improve binding. A good step-by-step introduction to humanization and demonstrating how conventional humanization has become in the art is provided on the internet http://www.cryst.bbk.ac.uk/˜ubcg07s/.

In general, at the same time, or during the process, the constant region would be switched from the murine IgM to another human constant region (such as a human IgM constant region, without or without the J-chain, as discussed above) to prepare a humanized chimeric antibody.

Additional Criteria for Antibody Therapeutics

As discussed herein, the function of the ABX-CBL antibody appears important to at least a portion of its mode of operation. By function, we mean, by way of example, the activity of the ABX-CBL antibody is CDC. Accordingly, it is desirable in connection with the generation of antibodies as therapeutic candidates against CD147 that the antibodies be capable of fixing complement and participating in CDC. There are a number of isotypes of antibodies that are capable of the same, including, without limitation, the following: murine IgM, murine IgG2a, murine IgG2b, murine IgG3, human IgM, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. patent application Ser. No. 08/730,639, filed Oct. 11, 1996), among others.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

By way of example, the 2.6.1 antibody discussed herein is a human anti-CD147 IgG2 antibody. If such antibody possessed desired binding to the CD147 molecule, it could be readily isotype switched to generate an human IgM, human IgG1, or human IgG3 isotype, while still possessing the same variable region (which defines the antibody's specificity and some of its affinity). Such molecule would then be capable of fixing complement and participating in CDC, in a similar manner to the ABX-CBL antibody.

Accordingly, as antibody candidates are generated that meet desired “structural” attributes as discussed above, they can generally be provided with at least certain of the desired “functional” attributes through isotype switching.

Design and Generation of Other Therapeutics

In accordance with the present invention and based on the activity of the ABX-CBL antibody with respect to CD147, it is now also possible to design other therapeutic modalities beyond ordinary antibody moieties, including, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

In connection with the generation of advanced antibody therapeutics, it may be possible to sidestep the dependence on complement for cell killing that we have demonstrated is necessary for the function of the ABX-CBL antibody through the use of bispecifics, immunotoxins, or radiolabels, for example.

For example, in connection with bispecific antibodies, bispecific antibodies can be generated that comprise (i) two antibodies one with a specificity to CD147 and another to a second molecule that are conjugated together, (ii) a single antibody that has one chain specific to CD147 and a second chain specific to a second molecule, or (iii) a single chain antibody that has specificity to CD147 and the other molecule. Such bispecific antibodies can be generated using techniques that are well known for example, in connection with (i) and (ii) see e.g., Fanger et al. Immunol Methods 4:72-81 (1994) and Wright and Harris, supra. and in connection with (iii) see e.g., Traunecker et al. Int. J. Cancer (Suppl.) 7:51-52 (1992). In each case, the second specificity can be made to the heavy chain activation receptors, including, without limitation, CD16 or CD64 (see e.g., Deo et al. 18:127 (1997)) or CD89 (see e.g., Valerius et al. Blood 90:4485-4492 (1997)). Bispecific antibodies prepared in accordance with the foregoing would be likely to kill cells expressing CD147, and particularly those cells in which the ABX-CBL antibody is effective.

In connection with immunotoxins, antibodies can be modified to act as immunotoxins utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g. Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each of immunotoxins and radiolabeled molecules would be likely to kill cells expressing CD147, and particularly those cells in which the ABX-CBL antibody is effective.

In connection with the generation of therapeutic peptides, through the utilization of structural information related to CD147 and antibodies thereto, such as the ABX-CBL antibody (as discussed below in connection with small molecules) or screening of peptide libraries, therapeutic peptides can be generated that are directed against CD147. Design and screening of peptide therapeutics is discussed in connection with Houghten et al. Biotechniques 13:412-421 (1992), Houghten PNAS USA 82:5131-5135 (1985), Pinalla et al. Biotechniques 13:901-905 (1992), Blake and Litzi-Davis BioConjugate Chem. 3:510-513 (1992). Immunotoxins and radiolabeled molecules can also be prepared, and in a similar manner, in connection with peptidic moieties as discussed above in connection with antibodies.

Assuming that the CD147 molecule (or a form, such as a splice variant or alternate form) is functionally active in a disease process, it will also be possible to design gene and antisense therapeutics thereto through conventional techniques. Such modalities can be utilized for modulating the function of CD147. In connection therewith the discovery of the present invention allows design and use of functional assays related thereto. A design and strategy for antisense therapeutics is discussed in detail in International Patent Application No. WO 94/29444. Design and strategies for gene therapy are well known. However, in particular, the use of gene therapeutic techniques involving intrabodies could prove to be particularly advantageous. See e.g., Chen et al. Human Gene Therapy 5:595-601 (1994) and Marasco Gene Therapy 4:11-15 (1997). General design of and considerations related to gene therapeutics is also discussed in International Patent Application No- WO 97/38137.

Small molecule therapeutics can also be envisioned in accordance with the present invention. Drugs can be designed to modulate the activity of CD147 based upon the present invention. Knowledge gleaned from the structure of the CD147 molecule and its interactions with other molecules in accordance with the present invention, such as the ABX-CBL antibody, CD46, CD55, CD59, and others can be utilized to rationally design additional therapeutic modalities. In this regard, rational drug design techniques such as X-ray crystallography, computer-aided (or assisted) molecular modeling (CAMM), quantitative or qualitative structure-activity relationship (QSAR), and similar technologies can be utilized to focus drug discovery efforts. Rational design allows prediction of protein or synthetic structures which can interact with the molecule or specific forms thereof which can be used to modify or modulate the activity of CD147. Such structures can be synthesized chemically or expressed in biological systems. This approach has been reviewed in Capsey et al. Genetically Engineered Human Therapeutic Drugs (Stockton Press, NY (1988)). Further, combinatorial libraries can be designed and synthesized and used in screening programs, such as high throughput screening efforts.

Therapeutic Administration and Formulations

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15^{th} ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Powell et al. “Compendium of excipients for parenteral formulations” PDA J Pharm Sci Technol. 52:238-311 (1998) and the citations therein for additional information related to excipients and carriers well known to pharmaceutical chemists.

### EXAMPLES

The following examples, including the experiments conducted and results achieved, are provided for illustrative purposes only and are not to be construed as limiting upon the present invention.

Experiment 1 Generation of Human Antibodies

Human antibodies were prepared in accordance with Mendez et al. Nature Genetics 15:146-156 (1997) and U.S. patent application Ser. No. 08/759,620, filed Dec. 3, 1996, the disclosures of which are hereby incorporated by reference herein in their entirety, through the immunization of XenoMouse™ animals with CEM cells, followed by fusions, and screening of the resulting hybridoma supernatants against CEM cells and in competition assays with the ABX-CBL antibody (Example 2).

Experiment 2 Immuno-affinity Purification of ABX-CBL Antigen

We undertook immunoaffinity purification of the antigen to which the ABX-CBL antibody bound. The antigen to which the CBL1 and ABX-CBL antibody bound appeared to be highly expressed on CEM cells. Immunoaffinity purification using the native ABX-CBL antibody was frustrated by the fact that the ABX-CBL antibody is an IgM antibody having a pentameric structure. Therefore, we prepared human IgG2 antibodies (Example 1), followed by fusions, and screening of the resulting hybridoma supernatants against CEM cells and tested for competition with the ABX-CBL antibody in binding assays with CEM cells using FACS. In the FACS competition assays, inhibition of the binding of ABX-CBL antibodies, labeled with FITC, to CEM cells was analyzed, both alone and in the presence of the anti-CEM human antibodies.

We obtained four hybridoma clones from the fusions that produced monoclonal antibodies that bound to the CEM cells and that were highly competitive with the ABX-CBL antibody in binding to the CEM cells. One hybridoma clone, designated 2.6.1 appeared most competitive.

We generated ascites to each of the four hybridoma clones, including the 2.6.1 hybridoma, in SCID mice and purified the 2.6.1 antibody using a Protein A affinity purification process using standard conditions. From the purified 2.6.1 antibody, we prepared an immunoaffinity column. To prepare the column, the purified 2.6.1 antibody was conjugated to CNBr activated Sepharose-4B, according to the manufacturer's specifications. Approximately 8.4 mg of the antibody was conjugated to about 2.0 g of the activated Sepharose. We passed cell lysates of CEM cells through the column and eluted the components that bound The elution product was analyzed by SDS-PAGE eletrophoresis, Western blotting, ELISAs, and BiaCore reactivity against CEM cell lysates.

The elution product that was purified from CEM cell lysates was demonstrated to be CD147 upon our sequencing of the diffuse band corresponding to 45-55 KD that we observed on Western Blot analysis after reaction with each of the 2.6.1 antibody and the ABX-CBL antibody. As will be observed from FIG. 1, the 2.6.1 antibody bound most intensely to a molecule or molecules contained within a diffuse band from about 45-55 KD, while the ABX-CBL antibody showed binding with a lower intensity to a similar band from about 45-55 KD.

Sequencing was accomplished upon a portion of the 45-55 KD band that was isolated through use of preparative gel electrophoresis and electroblotting techniques using a Perkin Elmer sequencer. We obtained a partial amino acid sequence of the molecule (between 35 through 40 residues). The resulting sequence information was analyzed through a protein database search (Protein Identification Resourse (PIR) R47.0, December 1995) and the sequence comparison data indicated that the molecule was CD147.

Western blots on CEM lysates were generally accomplished as follows:

CEM cells were homogenized in 10 mM Tris pH 7.5, 150 mM NaCl, 1% Triton X-100, and protease inhibitors to generate CEM extracts at 5×10⁸ cells/ml. The extract (5 μl) were electrophoresed on 12% SDS-PAGE gels and then blotted onto PVDF. The blot was cut into 5 strips in preparation for antibody staining. All first antibody staining was done at 14 g/ml in 1% gelatin/PBST buffer. All AP labeled second antibody was done at a dilution of 1:1000 in the same buffer. The rabbit-anti-mouse-hnRNP-k Protein antibody was supplied to us by Dr. Karol Bomstzyk at the University of Washington. Each of the ABX-CBL, Pharmingen, and 2.6.1 antibodies are described further herein.

Experiment 3 Purification of 62 KD Band

In order to purify the material contained in the 62 KD band, CEM whole cell lysates were prepared from approximately 3×10¹⁰ cells. The lysates were extracted and concentrated to provide about 3.8 mg of protein. A portion of the recovered protein was subjected to a series of chromatography steps: size exclusion, anion exchange, hydrophobic interaction, reversed phase, and microbore reversed phase. In each step, the fraction showing binding to the ABX-CBL antibody on Western blot was carried on to the next step. Following microbore reversed phase chromatography, approximately 5×10⁻⁶ grams of protein was recovered and a portion of the protein subjected to gel electrophoresis and electroblotting to generate approximately 90% pure 62 KD protein.

A direct N-terminal sequence was attempted, however, the molecule possessed a blocked N-terminus. Thus, the material was digested with CNBr and preparative gel electrophoresis and electroblotting were conducted, yielding bands at approximately 12 KD and 32.5 KD. The blotted fragments were sequenced and the resulting sequence results were analyzed through protein database searches (Protein Identification Resourse (PIR) R47.0, December 1995). The sequence comparison data indicated that the molecule was heterogeneous ribonuclear protein k (hnRNP-k), with the 12 KD band having residues 360 and up (after Methionine; 359) and the 32.5 KD band having residues 43 and up (after Methionine; 42).

Experiment 4 CD147 ELISA Assay

We have utilized the enriched purified antigen obtained from CEM cell lysates to develop a specific ELISA assay for the detection of the expression of CD147 in a secreted or membrane bound form. In the assay, we immobilize the CD147 antigen (for example, the CD147 antigen that is affinity purified from CEM cell lysates) in the wells of plates. Binding of the antigen can be accomplished using conventional techniques. Thereafter, the plates containing the antigen can be used for the detection of antibodies that are reactive with it using conventional techniques. We have demonstrated that each of the commercially available anti-CD147 antibodies (RDI-CBL535 (a murine anti-CD147 IgG2b antibody), available from RDI, Flanders, N.J., and 36901A (a murine anti-CD147 IgG1 antibody), available from Pharmingen, San Diego, Calif.), the ABX-CBL antibody, and the human antibodies that we have generated in Example 2 react specifically in this assay.

The present ELISA assay is useful as a screening system for detecting antibodies that bind to the CD147 antigen.

Experiment 5 Evidence Related to Role of 35 KD Band

As mentioned above, anecdotal evidence indicates that a 35 KD band could correspond to a singly-glycosylated form of CD147. See Kanekura et al. Cell Struct Funct 16.23-30 (1991). Further, it is also interesting to note that in comparisons of Western blots produced by two commercially available anti-CD147 antibodies (RDI-CBL535 (a murine anti-CD147 IgG2b antibody), available from RDI, Flanders, N.J., and 36901A (a murine anti-CD147 IgG1 antibody), available from Pharmingen, San Diego, Calif.) to the ABX-CBL and 2.6.1 antibodies indicates that each of the commercially available antibodies recognize a molecule that has a molecular weight around 35 KD and appearing similar to the 35 KD band recognized by the ABX-CBL antibody. See FIG. 1. Another interesting observation is that in the immunoaffinity purification mentioned above, when the effluent product from the 2.6.1 antibody was probed with the ABX-CBL antibody, the 35 KD band was no longer visible by Western blot. Rather, the ABX-CBL antibody appeared to bind to the diffuse band from 45-55 KD with relatively low intensity (similar to that shown in FIG. 1). This evidence indicates that the ABX-CBL antibody could bind preferentially to a different epitope on, or a different form of, CD147 than the 2.6.1 antibody and the commercially available antibodies.

Experiment 6 Complement Mediated Cell Killing

The UCLA group mentioned above (see e.g., U.S. Pat. Nos. 5,330,896 and 5,643,740) provided certain evidence that the CBL1 antibody operated through killing of certain activated cell populations while the antibody did not react with non-activated cells. For example, in a microcytotoxicity assay, the CBL1 antibody was disclosed to kill activated lymphocytic cells but not other normal cells.

In connection with this experiment, the following materials and procedures were utilized:

Mixed Lymphocyte Reaction

Mixed lymphocyte reaction (MLR) is an in vitro system for assaying T lymphocyte proliferation in cell-mediated responses. A cell-mediated response is an in vitro assay of effector cytotoxic function, which can also be assayed in vivo by graft-versus-host reaction in experimental animals. When co-culturing allogeneic lymphocytes in MLR the cells undergo extensive blast transformation and cell proliferation. Thus, MLR can be quantified by adding tritium-labeled thymidine ([³H]thymidine) to the culture medium and monitoring uptake of label into DNA of the dividing lymphocytes.

To determine the function and quality CBL-1 and ABX-CBL antibody we used MLR to test the ability of CBL-1 and ABX-CBL to inhibit lymphocyte proliferative responses. Peripheral blood mononuclear cells were isolated from two HLA mismatched individuals by Ficoll-Paque gradient centrifuigation. Allogeneic lymphocytes were mixed (1:1) and co-cultured (total of 5×10⁵ cells/well in 96-well plate) in vitro for six days. Lymphocytes from one individual were irradiated with 3000 rads prior to the culture. CBL-1 and ABX-CBL antibody plus either 10% rabbit or 25% human complement were added to the culture 24 h prior to the end of the culture. The culture was pulsed with [³H]methyl-thymidine (Amersham) overnight and harvested on day 6. Lymphocyte proliferative response was determined by measuring [³H]-thymidine incorporation. Percentage inhibition was calculated as the cpm in the absence of antibody minus the cpm in the presence of antibody divided by the cpm in the absence of antibody.

ConA Stimulated Lymphocyte Proliferation

Human PBMC were isolated as described above and stimulated by the mitogen Concanavalin A (ConA) at 5 ug/ml for 48 h. Antibodies with or without 10% complement were added to the culture 24 h prior to the end of the culture. The culture was pulsed with [³H]-methyl-thymidine overnight and harvested next day. Lymphocyte proliferative response was determined by measuring [³H]-thymidine incorporation. Percentage inhibition was calculated as the cpm in the absence of antibody minus the cpm in the presence of antibody divided by the cpm in the absence of antibody.

FACS Analysis of Cell Surface Molecules

For cell surface expression of different surface molecules, immunofluorescent staining and analysis on a FACSvantage (Becton Dickinson, San Jose, Calif.) have been described (FACScan Manual. Becton Dickinson, San Jose, Calif.). Monoclonal antibodies anti-CD3-PE, anti-CD4-PE, anti-CD8-PE, anti-CD14-PE, anti-CD20-PE, anti-CD25-FITC and anti-CD25-PE were obtained from Becton Dickinson. Anti-CD55-FITC and anti-CD59-FITC were purchased from Pharmingen (San Diego, Calif.). ABX-CBL and cem2.6.1 were conjugated with FITC and PE, respectively, at Abgenix.

Complement-dependent Cytotoxicity Assay Using Alamar Blue

Complement-dependent cytotoxicity (CDC) assay was performed as described (Gazzano-Santoro et al. “A non-radioactive complement-dependent cytotoxicity assay for anti-CD20 monoclonal antibody” J. Immunol. Methods 202:163-171 (1997). Fifty microliters of a cell suspension of 10⁶ cells/ml, 50 μl of various concentrations of antibodies and 50 μl of a 10% rabbit or human complement were added to flat-bottomed 96-well tissue culture plate and incubated for 2 hours at 37° C. and 5% CO₂. Fifty microliters of Alamar blue (Accumed International) were then added (final 10%) and the incubation continued for another 5 hours. The plates were allowed to cool to room temperature for 10 minutes on a shaker and the fluorescence was read using a 96-well fluorometer with excitation at 530 nm and emission at 590 nm. Results were expressed in relative fluorescence units (RFU).

In our work, we have demonstrated that CBL1 and ABX-CBL operate through complement mediated cell killing. Use of the CBL1 antibody by itself, the isotype-matched control mouse IgM antibody by itself (FIG. 2), or complement (either human or rabbit) by itself in the MLR or modified MLR assay (ConA induced lymphocyte proliferation assay) is ineffective in inhibiting T-cell proliferation. See FIGS. 2-5. However, when both complement and the CBL1 or ABX-CBL antibody are present, T-cell proliferation is inhibited in a dose dependent manner. See FIGS. 2-5. The human IgG2 antibody 2.6.1 is ineffective in inhibiting T-cell proliferation in the same assay, either by itself, or in combination with complement. See FIG. 5. This is expected, since the 2.6.1 antibody as a gamma-2 is notoriously less efficient in complement mediated lysis than is an IgM antibody, such as the ABX-CBL antibody.

The combination of CBL1 or ABX-CBL and complement only kill activated T-cells (both CD4⁺ and CD8⁺), activated B-cells, and monocytes, but does not effect resting T-cells and B-cells because such cells do not express CD147. It is important, to note that monocytes are also killed by ABX-CBL and complement. This data provides an explanation for the operation of ABX-CBL therapy in diseases, such as GVHD, because, ABX-CBL selectively depletes those effector cells (activated T- and B-cells) and the antigen presenting cells (monocytes and B-cells) which ordinarily would lead to fuirther T-cell activation.

Experiment 7 Evidence Related to Cellular Activation

Using techniques described in Experiment 6, we also demonstrated that the CD25 marker appears to be expressed in high levels in the same cellular populations as those expressing the antigen to which the ABX-CBL antibody binds. See FIG. 6. This finding provided a useful marker to detect whether the cells expressing CD25 were depleted in connection with the MLR assay. Where the MLR assay is conducted utilizing a variety of activated cell populations, CD25 expressing cell populations are depleted only in those treated with the ABX-CBL antibody plus complement. See FIGS. 7-11. The selective killing of different cell populations are shown in FIGS. 10-12.

Experiment 8 Evidence Related to the Role of Expression Levels of CD147

We have also considered whether CD147 expression levels are higher in given populations of cells (which could also be relevant to CDC).

In flow cytometry studies with peripheral blood mononuclear cells (PBMC) with the ABX-CBL antibody, we have noticed that, prior to the addition of complement, there are populations of cells that appear to express high and low levels of CD147. After complement is added, there are populations of cells that appear to correspond to the low level expressers mentioned above. It appears that these results could be indicative of density of CD147 expression levels on the cell surface. Density can play a role in CDC through providing additional antigen binding sites to allow for distortion of the antibody which is the first step in triggering the complement cascade. Upon distortion of the antibody, the factor clq binds first and the cascade proceeds.

Whether the expression level (or, density) of CD147 in cellular populations plays a role in the therapeutic efficacy of the ABX-CBL antibody can be assayed through analyzing the expression levels of the CD147 molecule in various cellular populations. Generally, the experiments are conducted where beads having various known quantities of the CD147 antigen on their surface are prepared and analyzed on FACS (i.e., utilizing a FITC-labeled anti-CD147 IgG antibody) in order to generate approximately 10-20 data points of different quantities of antigen on the beads. A linear regression curve is prepared from such data. Thereafter, cells expressing the CD147 antigen can be run through FACS and the relative quantities of antigen on the surface of the cells can be calculated from the linear regression curve.

Experiment 9 Evidence Related to the Role of Complement Inhibitory Molecules

Further, in order to consider the cellular specificity of the mode of operation of the ABX-CBL antibody, we investigated various cells to which the ABX-CBL antibody binds and considered whether such cells were killed in a manner similar to complement mediated lysis. In connection with this work, we have investigated various cells to which the ABX-CBL antibody binds and considered whether such cells were (i) killed and (ii) if so, was the mechanism similar to complement mediated lysis. In the experiment, we looked for ABX-CBL antibody binding to a number of cells (and, thus, the antigen to which the ABX-CBL antibody binds is expressed upon such cells). Those cells to which ABX-CBL would bind were then tested for complement mediated lysis through treatment with the ABX-CBL antibody and complement. Two T-cell lines (CEM and Jurkat cells), a monocyte line (U937 cells), and three tumor cell lines (A431 (epidermal), SW948 (colon), and MDA468 (breast)), each of which bound the ABX-CBL antibody were examined. Despite the expression on such cells lines, the ABX-CBL antibody is very specific about which cells are killed, being restricted to the CEM T-cell line and U937 monocyte line. See FIG. 13. We also analyzed two endothelial cell lines (i) ECV-304 (ATCC CRL-1998) is a spontaneously transformed immortal EC established from the vein of an apparently normal human umbilical cord and carrying EC characteristics and (ii) HUV-EC-C (ATCC CRL-1730) is an EC line derived from the vein of a normal human umbilical cord. Using FACS, we found that each of the ECV-304 and HUVEC-C lines stained positive against the 2.6.1, Pharmingen, and ABX-CBL antibodies suggesting that these ECs do express CD147 on the surface. FIGS. 15 and 16, respectively. We then carried out in vitro Alamar-blue based CDC assay and demonstrated that both EC lines were resistant to ABX-CBL mediated CDC in the presence of human complement. See FIGS. 17 and 18, respectively.

In order to farther understand why cells that all appear to express CD147 would not be killed by the ABX-CBL antibody in the presence of complement, we looked into CD46, CD55, and CD59 expression in such cells. Each of CD46 (membrane cofactor protein, MCP), CD55 (decay accelerating factor, DAF), and CD59 (membrane attack complex inhibitor, MACI) have been implicated as complement inhibitory molecules. See e.g., Liszewski et al. Annu. Rev. Immunol. 9:431 (1991) and Loveland et al. “Coordinate functions of multiple complement regulating molecules, CD46, CD55 and CD59” Transpl. Proc. 26:1070 (1994) related to CD46, Kinoshita et al. “Distribution of decay-accelerating factor in the peripheral blood of normal individuals and patients with paroxysmal nocturnal hemoglobinuria” J. Exp. Med. 162:75 (1985) and Loveland et al. “Coordinate functions of multiple complement regulating molecules, CD46, CD55 and CD59” Transpl. Proc. 26:1070 (1994) related to CD55, and Whitlow et al. “H19, a surface membrane molecule involved in T-cell activation, inhibits channel formation by human complement” Cell. Immunol. 126: 176 (1990), Loveland et al. “Coordinate functions of multiple complement regulating molecules, CD46, CD55 and CD59” Transpl. Proc. 26:1070 (1994), and Davies, A. and Lachmann, P. J. “Membrane defense against complement lysis: the structure and biological properties of CD59” Immunol. Res. 12: 258 (1993) related to CD59. Accordingly, we considered whether there was differential expression of either, or both, of these molecules on the cell lines tested above. Indeed, all of the cells, except the CEM line and the U937 line, expressed both of the molecules. And, indeed, the endothelial cell lines HUVEC-C and ECV-304 expressed all three, CD46, CD55, and CD59. FIGS. 19 and 20, respectively. In contrast, the CEM line expressed only CD59 and the U937 line expressed only CD55. See FIG. 14. This data is useful in connection with the prediction of cells that could be selectively eradicated by ABX-CBL and consequently targeted in connection with anti-CD147 in accordance with the present invention.

Experiment 10 Cloning and Expression of CD147 in Eukaryotic Cells and Binding of Antibodies

In the present experiment, we cloned fuill length CD147 cDNA through use of PCR in connection with the Jurkat Zapp Express phagemid DNA (Stratagene).

The following PCR primers were utilized, based on the CD147 sequence reported by Miyauchi et al. J. Biochem. 110:770-774 (1991) (Gene Bank Accession No. D45131):

| | | |
|---|---|---|
| 5′: | 5′-GACTACGAATTCTTGTAGGACCGGCGAGG | (SEQ ID NO:42) |
| | AATAGG-3′ | |
| 3′: | 5′GACTACGGGCCCGGTGAGAACTTGGAATCT | (SEQ ID NO:43) |
| | TGCAAGC-3′ | |

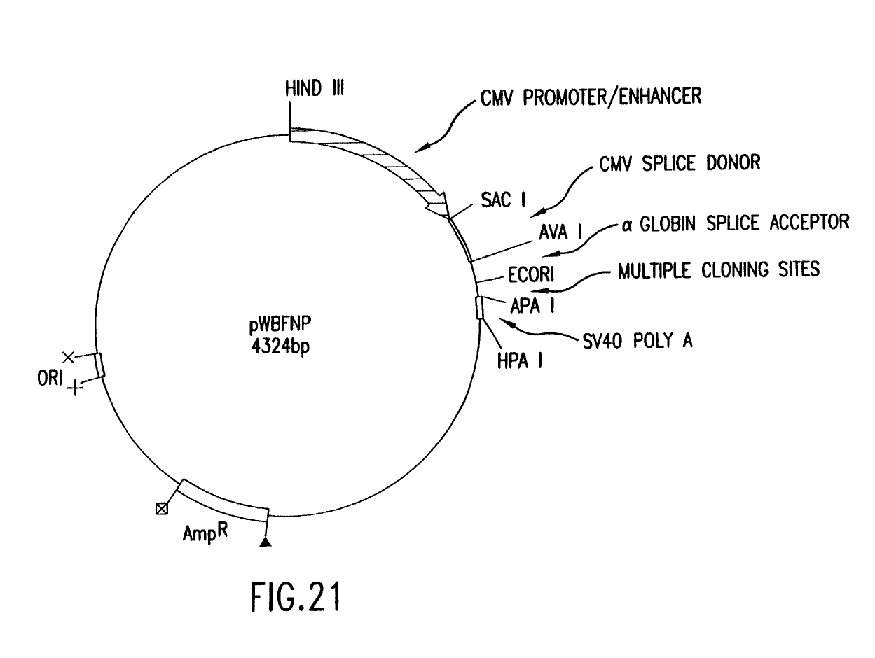

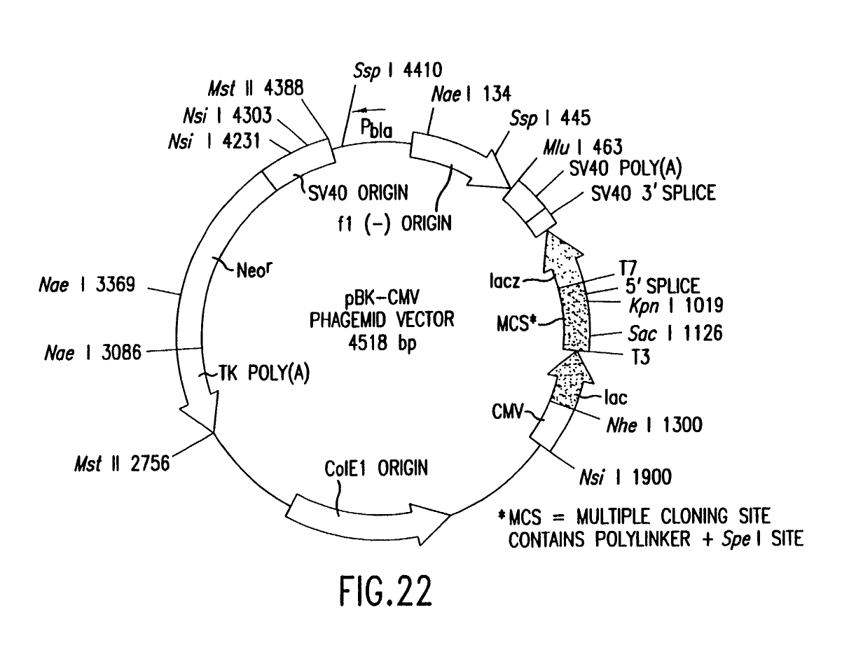

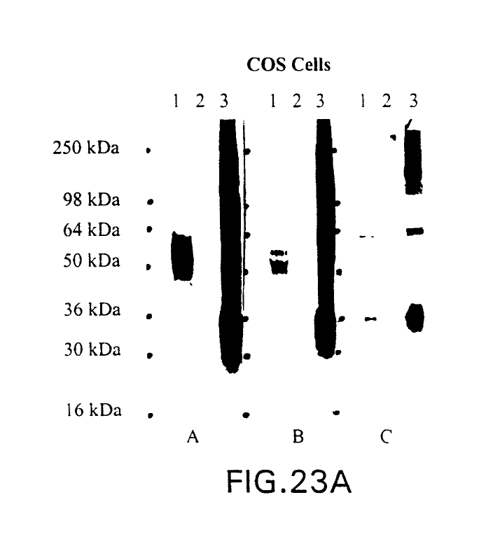

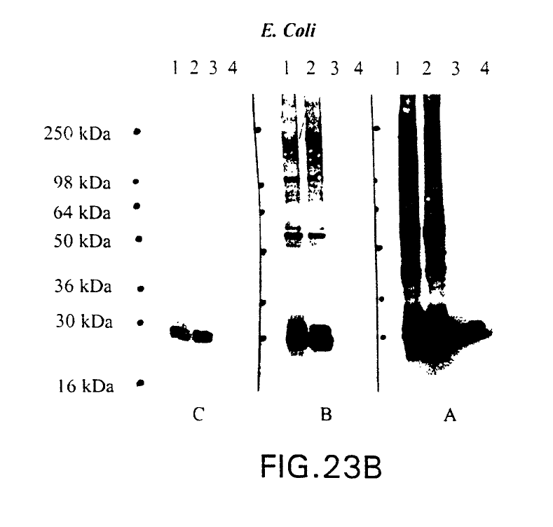

A 949 base pair PCR product was isolated whose open reading frame encoded the 269 amino acid CD147 protein. The PCR product was digested with EcoR1 and Apal and ligated into the EcoR1 and Apa1 sites of mammalian expression vectors pWBFNP (FIG. 21) and pBKCMV (Stratagene) (FIG. 22) (digested with NheI/Spel to remove the lac promoter and the lacZ ATG between positions 1300 and 1098) to create the vectors CD147/pWBFNP and CD147/pBKCMV(delta-Nhel/SpeI) respectively. In the constructs, eukaryotic expression of CD147 is driven from the cytomegalovirus (CMV) immediate early promoter. CD147/pWBFNP, CD147/pBKCMV(delta-NheI/SpeI) and control vectors pWBFNP and pBKCMV were transiently transfected into monkey kidney (COS-7) cells by the CAPO₄ method. Cells were harvested 60 hours later, washed in PBS and stained with anti-ABX-CBL-FITC, anti-CEM2.6.1./anti-HulgG-FITC, or anti-CD147-FITC (Pharmingen) and analyzed by FACS analysis and Western blot analysis (see FIG. 23A). The blot was accomplished using procedures described in Example 3.

FACS analysis revealed an increase in specific cell surface staining with all three antibodies only on COS cells transfected with vectors expressing CD147 cDNA (CD147/pWBFNP and CD147/pBKCMV (delta-NheI/Spel)). COS cells transfected with CD147 cDNA showed binding to each of the antibodies in each of the FACS and Western blot analyses. In contrast, COS cells transfected with control vectors were negative for binding with each of the 2.6.1 and ABX-CBL antibodies. With respect to the Pharmingen antibody, certain background staining was observed in cells transfected with control vectors on FACS and no binding on Western blot analysis. The transfected cells showed significant binding over background on FACS and were positive on Western blot analysis. Our results confirm that the ABX-CBL and the 2.6.1 antibodies bind to CD147.

Experiment 11 Cloning and Expression of CD147 in Eukaryotic Cells and Binding of Antibodies

Utilizing a slightly modified vector, we also transfected E. coli cells with the CD147 cDNA. In the experiment, CD147 cDNA generated as above was subdloned into pBKCMV (Stratagene) (FIG. 22). CD147/pBKCMV plasmid DNA was transformed into E.coli strain XL1-Blue MRF′ (Strategene). Cultures were grown in LB media supplemented with kanamycin at 50 μg/ml to OD₆₀₀ of 0.7 then for an additional 3 hours in the presence of 1 mM isopropyl-B-D-thio-galactopyranoside (IPTG). Cells were harvested by centrifugation and stored frozen at −20° C. The E. coli cells so transfected were capable of expression of the CD147 molecule as evidenced by Western blotting analysis of each of the ABX-CBL, 2.6.1, and Pharmingen antibodies. Since the prokaryotic E. coli cells should not glycosylate the expressed CD147, it was expected that the molecular weight of the CD147 expressed by the E. coli should closely approximate the predicted, unglycosylated molecular weight of CD147 of about 27 KD. Indeed, in each case, binding of the three antibodies on Western blot analysis was observed to a band between about 27 and 30 KD. FIG. 23B. The blot was accomplished using procedures described in Example 3.

This data further confirms that the ABX-CBL and the 2.6.1 antibodies bind to CD147. Further, the evidence indicates that ABX-CBL binding to CD147 is not directly based on carbohydrate binding, i.e., that ABX-CBL does not bind directly to a carbohydrate epitope on CD147. Such data, however, does not eliminate the possibility that binding to CD147 is influenced by the presence of carbohydrate or glycosylation.

Experiment 12 Epitope Analysis

In order to further elucidate the binding of the ABX-CBL antibody to CD147, we undertook phage display experiment. Such experiments were conducted through panning a phage library expressing random peptides for binding with the ABX-CBL and 2.6.1 antibodies to determine if we could isolate peptides that bound. If successful, certain epitope information can be gleaned from the peptides that bind.

In general, the phage libraries expressing random peptides were purchased from New England Biolabs (7-mer and 12-mer libraries, Ph.D.-7 Peptide 7-mer Library Kit and Ph.D.-12 Peptide 12-mer Library Kit, respectively) based on a bacteriophage M13 system. The 7-mer library represents a diversity of approximately 2.0×10⁹ independent clones, which represents most, if not all, of the 20⁷=1.28×10⁹ possible 7-mer sequences. The 12-mer library contains approximately 1.9×10⁹ independent clones and represents only a very small sampling of the potential sequence space of 20¹²=4.1×10¹⁵ 12-mer sequences. Each of 7-mer and 12-mer libraries were panned or screened in accordance with the manufacturer's recommendations in which plates were coated with an antibody to capture the appropriate antibody (goat anti-human IgG Fc for the 2.6.1 antibody and goat anti-mouse μ chain for the ABX-CBL antibody) followed by washing. Bound phage were eluted with 0.2 M glycine-HCl, pH 2.2. After 3 rounds of selection/amplification at constant stringency (0.5% Tween), through use of DNA sequencing, we characterized a total of 5 clones from the 7-mer library and 6 clones from the 12-mer library reactive with the ABX-CBL antibody and a total of 6 clones from each of the 7-mer and 12-mer libraries reactive with the 2.6.1 antibody. Reactivity of the peptides was determined by ELISA. For an additional discussion of epitope analysis of peptides see also Scott, J. K. and Smith, G. P. Science 249:386-390 (1990); Cwirla et al. PNAS USA 87:6378-6382 (1990); Felici et al. J. Mol. Biol. 222:301-310 (1991), and Kuwabara et al. Nature Biotechnology 15:74-78 (1997).

No consensus sequence was readily apparent for reactivity of the 2.6.1 antibody with CD147. However, sequence alignment of the characterized 7-mer and 12-mer sequences against the amino acid sequence of CD147 yielded a number of matches for a single sequence within CD147 from residue number 177 through residue number 188 (ITLRVRSH (SEQ ID NO:1)). In particular, each of the 7-mers contained sequence matches (represented by *) to 3 or more residues within this sequence of CD147:

7-mer sequences

| | | |
|---|---|---|
| | * * * | |
| 1. | EE RLR S Y | (SEQ ID NO:2) |
| | *** | |
| 2. | YE RVR W Y | (SEQ ID NO:3) |
| | * * * | |
| 3. | EE RLR S Y | (SEQ ID NO:4) |
| | * * * | |
| 4. | AE RIR S I | (SEQ ID NO:5) |
| | * * * | |
| 5. | EE RLR S Y | (SEQ ID NO:6) |

Further, 4 of the 12-mers contained sequence matches (represented by *) to 3 or more residues within this sequence of CD147, with 4 matches for 12-mer peptide number 1 and for 6 matches of 12-mer peptide number 2:

12-mer sequences

| | | |
|---|---|---|
| | * * * * | |
| 1. | TVHGDL RLR S LP | (SEQ ID NO:7) |
| | * * * * * * | |
| 2. | TNDIGL RQR S HS | (SEQ ID NO:8) |
| | * * * | |
| 3. | SPLLDGQ RER S Y | (SEQ ID NO:9) |
| | * * * | |
| 4. | YDLPM RSR S YPG | (SEQ ID NO:10) |
| | * | |
| 5. | SLAPLWY YSR H G | (SEQ ID NO:20) |
| 6. | HTPETAPLPATV | (SEQ ID NO:21) |
| | | (no binding) |

These results indicate a consensus sequence of RXRS (SEQ ID NO:11) that is present in 10 of the sequenced clones. Accordingly, we had a synthetic peptide prepared (AnaSpec Incorporated, San Jose, Calif.) which spanned residues 169-183 of CD147 with the following sequence (with —OH representing carboxy terminus):

| KGSDQAIITLRVRSH-OH | (SEQ ID NO:12) |
|---|---|
| \| \| | |
| 170 184 | |

Below, the amino acid sequence of CD147 is provided with the 15-mer peptide's sequence indicated by double underlining and the RXRSH (SEQ ID NO:13) consensus sequence indicated in bold. In addition, putative N-linked glycosylation sites of CD147 are shown as underlined and italics:

CD147 Sequence

(SEQ ID NO:14)

MAAALFVLLGFALLGTHGASGAAGTVFTTVEDLGSKILLTCSLNDSATEV
TGHRWLKGGVVLKEDALPGQKTEFKVDSDDQWGEYSCVFLPEPMGTANIQ
LHGPPRVKAVKSSEHINEGETAMLVCKSESVPPVTDWAWYKITDSEDKAL
MNGSESRFFVSSSQGRSELHIENLNMEADPGQYRCNGTSSKGSDQAIITL [symbol]
LAALWPFLGIVAEVLVLVTIIFIYEKRRKPEDVLDDDDAGSAPLK
SSGQHQNDKGKNVRQRNSS

The 15-mer peptide was assayed using ELISA and it was determined that the ABX-CBL antibody specifically bound to the peptide. Further, neither the 2.6.1 antibody nor a control murine IgM antibody bound to the peptide. However, based on a competition study between the CD147 antigen and the 15-mer peptide, the ABX-CBL antibody's binding to the 15-mer peptide can only be measured when the 15-mer peptide is coated on plates and not when the peptide is in solution. Indeed, in competition experiments in which the ABX-CBL antibody is bound to either the peptide or the CD147 antigen coated to plates, the ABX-CBL antibody is not removed or replaced by the peptide in solution even at high concentrations. Nevertheless, the binding of the ABX-CBL antibody to the 15-mer peptide can be specifically competed by the CD147 antigen and positive phage preparations mentioned above but not with non-specific antigen (i.e., L-Selectin isolated from cell membrane or human plasma) or the negative phage preparations mentioned above. Similarly, the binding of the ABX-CBL antibody to the CD147 antigen can be specifically competed by positive phage preparations as compared to negative phage preparation in competition assays using preincubation.

These results indicate that while the sequence within CD147 that contains the consensus sequence RXRSH is important to the binding of the ABX-CBL antibody to CD147, it does not fully explain ABX-CBL's binding to CD147. Indeed, the data also suggests that the consensus sequence contained either in the 15-mer peptide when bound to the plate or the reactive phage materials when tethered to the phage coat protein binds more tightly to the ABX-CBL antibody than does the free peptide in solution. Taken together, while not wishing to bound to any particular theory or mode of operation, it is possible that CD147 possesses certain conformations that are not well mimicked in the 5-mer peptide in solution. Nevertheless, the above epitopic information is important to understanding the manner in which the ABX-CBL antibody binds to CD147 and to producing other candidate molecules against CD147 as a therapeutic target.

It is interesting to note that in addition to the results above in connection with the presence of the RXRSH consensus sequence within CD147, we also looked for the presence of the consensus sequence within the hn-RNP-k protein to which ABX-CBL also appears to bind. Such analyses were conducted by sequence alignment against the phage derived peptides discussed above. Two sequences were found which possessed statistically interesting matches:

First, there was a match (indicated by *) of 5 amino acids with the 7-mer peptide number 4:

| * ** ** | (SEQ ID NO:15) |
|---|---|
| PE RIL SI | |
| \| | |
| 84 | |

Second, there was a match (indicated by *) of 5 amino acids with the 12-mer peptide number 1:

| * * * ** | (SEQ ID NO:16) |
|---|---|
| GGS RAR NLP | |
| \| | |
| 300 306 | |

The amino acid sequence of the hn-RNP-k protein is provided below with such sequences indicated by double underlining. In addition, a number of RXR sequence motifs are present in the hn-RNP-k protein's sequence which are also indicated by underlining:

hn-RNP-k Protein Sequence

(SEQ ID NO:17)

METEQPEETFPNTETNGEFGKRPAEDMEEEQAFKRSRNTDEMVELRILLQ
SKNAGAVIGKGGKNIKALRTDYNASVSVPDSSGPERILSISADIETIGEI
LKKIIPTLEEGLQLPSPTATSQLPLESDAVECLNYQHYKGSDFDCELRLL
IHQSLAGGIIGVKGAKIKELRENTQTTIKLFQECCPHSTDRVVLIGGKPD
RVVECIKIILDLISESPIKGRAQPYDPNFYDETYDYGGFTMMFDDRRGRP
VGFPMRGRGGFDRMPPGRGGRPMPPSRRDYDDMSPRRGPPPPPPGRGGRG
GSRARNLPLPPPPPPRGGDLMAYDRRGRPGDRYDGMVGF
SADETWDSAIDTWSPSEWQMAYEPQGGSGYDYSYAGGRGSYGDLGGPIITT
QVTIPKDLAGSIIGKGGQRIKQIRHESGASIKIDEPLEGSEDRIITITGT
QDQIQNAQYLLQNSVKQYSGKFF

Without wishing to be bound to any particular theory or mode of operation, it is possible that the binding of the ABX-CBL antibody to the hn-RNP-k protein is partially explained by the presence of these motifs within the protein.

Experiment 13 Expression of CD147 and Binding of Antibodies

Indeed, the desirability of mimicking ABX-CBL binding and efficacy is highlighted based upon a preliminary tissue distribution study of the ABX-CBL antibody. In the study, ABX-CBL is widely distributed throughout a variety of tissues. However, the majority of the distribution is likely to be due to nonspecific binding. Nevertheless, there appears to be specific binding in endothelial cells (venules, arterioles, but not capillary beds), smooth muscle, and some mesothelium. Also, the lymphoreticular tissues appear to be bound, although, the staining seems to be restricted to large lymphocytes, presumably activated blasts. From the study conducted, it was difficult to distinguish intracellular from extracellular staining. A certain amount of cytoplasmic staining was clearly evident and could have been related to hn-RNP-k binding.

Experiment 14 Analysis of Activity of MOPC21 Light Chain Activity in ABX-CBL Antibody

Two different techniques were utilized to endeavor to study the role of the MOPC21 light in ABX-CBL activity. In each technique, efforts were made to segregate the MOPC21 light chain from the cell line producing the IgM antibody. In the first technique, segregation was effected by fusion of the ABX-CBL IgM producing cell line with another cell line (NSO). In the second technique, segregation by spontaneous loss variants was endeavored. The fusion technique was successful and work was stopped on the second technique.

In the fusion technique, in general, NSO cells were transfected with a puromycin containing vector to create a puromycin⁻ NSO cell line. The ABX-CBL IgM producing cell line was grown in HAT medium was fused with the puromycin⁺ NSO cell line.

In general, fusions are accomplished in accordance with the following techniques and procedures:

Preparation of Cells

Prior to fusion, parental cell lines for use in the fusion are grown up and maintained in medium containing DMEM high, 10% FBS, 1% non-essential amino acids, 1% pen-strep, and 1% L-glutamine.

On the day prior to fusion, each of the parental cell lines are prepared and split to provide a cell density of approximately 10⁵ cells/ml. On the day of the fusion, cells are counted and the fusion is commenced when, and assuming, that cell count for each of the parental cell lines are within the range of about 1.5-2.5×10⁵ cells/ml. Sufficient quantities of each of the parental cell lines to make up 5×10⁶ cells each are withdrawn from the cultures and added to a 50 ml centrifugation tube and the cells are pelleted at 1200 rpm for approximately 5 minutes. Concurrently with the preparation of the cells, incomplete DMEM, PEG, and double selection media are prewarmed in an incubator bath. Following pelleting, cells are resuspended in 20 ml incomplete DMEM and pelleted again. Thereafter, the cells are resuspended in 5 ml incomplete DMEM and the two parental cell lines are pooled in a single tube and pelleted again to form a co-pellet containing both of the parental cell lines. The co-pellet is resuspended in 10 ml incomplete DMEM and again pelleted. All of the supernatant is then removed from the co-pellet and the cells are ready for fusion.

Fusion

Following removal of all of the supernatant, 1 ml PEG- 1500 is added over the course of I minute to the co-pellet while stirring. After addition of the PEG is completed, either gentle stirring with a pipet is continued for 1 minute or the suspended co-pellet can be allowed to stand for 1 minute. Thereafter, 10 ml of incomplete DMEM is added to the co-pellet over the course of 5 minutes with slow stirring. The mixture is then centrifuged at about 1200 rpm for 5 minutes and following centrifugation, the supernatant is aspirated off, and 10 ml of complete double selection medium is added and gently stirred into the cells. The cells are then plated at 100 μl/well into 10 96-well microtiter plates and placed into an incubator (37° C. with 10% CO₂) where they are not disturbed for 1 week. After the passage of a week, plates are fed by adding 100 μl of complete double selection medium to each well.

Double selection medium is prepared depending upon the marker gene utilized in connection with the parental cell lines. In the majority of our experiments, the selectable markers conferring puromycin, hygromycin, of hypoxanthine and thymidine resistance are utilized. Concentrations required to obtain complete cell killing of NS/0 cells were determined through use of kill curves and resulted in our use of 6 micrograms/ml of puromycin and 350 micrograms/ml of hygromycin. In connection with HPRT resistance, we used HAT media supplement from Sigma using standard conditions.

In the present case, cells were selected for puromycin⁺/HAT resistance. Individual clones were picked based on selection and clones were expanded in 96-well plates. Plates were split (½ for freezer stock, ½ for growth). Total RNA was isolated from the growth plates using the Qiagen 96-well RNA isolation kit according to the manufacturer's instructions. Primers were designed based on conserved sites on the MOPC21 and the ABX-CBL kappa chains that would amplify fragments of the chains which contained unique restriction sites in the respective chains, as follows:

| Restriction site | Chain | Position |
|---|---|---|
| AgeI (BsrFI) | MOPC21 | 135 |
| BstYI | MOPC21 | 173 |
| KpnI | ABX-CBL | 85 |
| NsiI | ABX-CBL | 130 |
| XcmI | MOPC21 | 58 |

| | | |
|---|---|---|
| 5 prime: | 5′-GCA GTC TCC TAA ACT GCT | (SEQ ID NO:44) |
| | positions 99-116 allows | |
| | analysis of BstYI | |
| | restriction site; | |
| or | | |
| 5 prime: | 5′-ACC TGC AAG GCC AGT | (SEQ ID NO:45) |
| | positions 40-54 allows | |
| | analysis of NsiI or KpnI | |
| | restriction sites, | |
| 3 prime: | 5′-CAC TCA TTC CTG TTG AAG. | (SEQ ID NO:46) |

Accordingly, through amplification with the above primers, followed by digestion with the appropriate restriction enzymes, presence or absence of MOPC21 or ABX-CBL could be readily detected on agarose gel electrophoresis. Through use of the above techniques, at least 6 variants were obtained that lost the MOPC21 light chain expression but retained the ABX-CBL kappa. No variants were directly obtained that lost ABX-CBL kappa chain expression and retained the MOPC21 chain expression. However, we isolated a cell line that appeared to be a minimal producer of ABX-CBL light chain and subcloned the line. It turned out to be a mixed cell line of a heterogeneous MOPC21/ABX-CBL light chain producer and a MOPC21 light chain only producer. Accordingly, we isolated the MOPC21 only producer after subcloning.

MOPC21 only light chain containing and ABX-CBL only light chain containing antibodies were compared and supported the conclusion that the presence or absence of the MOPC21 light chain did not appear to substantially impact antibody binding or properties of the antibodies. Although, it did appear that the MOPC21 only light chain containing antibody did not bind as intensely on Western blotting to CEM cells or CD147.

Experiment 15 Generation and Characterization of Human Antibodies to CD147

In accordance with Experiment 1, we generated a panel of fully human anti-CD147 antibodies. Antibodies were screened by ELISA for binding with CD147 and FACs for ability to compete with ABX-CBL. Certain of such antibodies were sequenced. The sequences of certain of the antibodies were compared to transcripts of the germline V-gene segments to somatic mutations in the amino acid sequences. Such sequence comparisons are shown in FIGS. 44 through 46. cDNA sequences and protein transcripts of and for each of the antibodies are shown in FIGS. 24 through 33. In addition, CDRs, according to Kabat numbering scheme, of the heavy chains and kappa light chains of the antibodies are shown in FIGS. 34 through 43.

In view of a number of tests that were conducted, particularly, competition studies between ABX-CBL and the certain of the antibodies, the 2.6.1 IgM antibody was chosen for additional development.

Experiment 16 Generation of 2.6.1 Expression Vectors for the Generation of IgG1, IgM, and Multimeric IgM Antibodies

In order to investigate the ability of the 2.6.1 antibody to operate in ADCC, similar to the CBL1 and ABX-CBL antibodies, we were interested in preparing IgM and IgG1 isotypes of the 2.6.1 antibody. The isotype switching of the 2.6.1 antibody from an IgG2 to an IgG1 was relatively simple. Whereas, the switching of the 2.6.1 antibody to a multimeric IgM required certain additional steps.

As will be appreciated, all of the IgMs that were generated from XenoMouse animals were monovalent. Accordingly, in order to prepare a fully human multimeric IgM antibody, we first were required to clone the human J-chain gene. from human buffy coat cells. The sequence of the human J-chain cDNA is shown below with the 5′-untranslated portion shown in bold, italics and underlining:

| (SEQ ID NO:47) | |
|---|---|
| [symbol] ATGAAGAACC ATTTGCTTTT CTGGGGAGTC | 50 |
| CTGGCGGTTT TTATTAAGGC TGTTCATGTG AAAGCCCAAG AAGATGAAAG | 100 |
| GATTGTTCTT GTTGACAACA AATGTAAGTG TGCCCGGATT ACTTCCAGGA | 150 |
| TCATCCCTTC TTCCGAAGAT CCTAATGAGG ACATTGTGGA GAGAAACATC | 200 |
| CGAATTATTG TTCCTCTGAA CAACAGGGAG AATATCTCTG ATCCCACCTC | 250 |
| ACCATTGAGA ACCAGATTTG TGTACCATTT GTCTGACCTC TGTAPAAAAT | 300 |
| GTGATCCTAC AGAAGTGGAG CTGGATAATC AGATAGTTAC TGCTACCCAG | 350 |
| AGCAATATCT GTGATGAAGA CAGTGCTACA GAGACCTGCT ACACTTATGA | 400 |
| CAGAAACAAG TGCTACACAG CTGTGGTCCC ACTCGTATAT GGTGGTGAGA | 450 |
| CCAAAATGGT GGAAACAGCC TTAACCCCAG ATGCCTGCTA TCCTGACTAA | 500 |

The J-chain gene encodes the human J-chain with the following sequence.

| (SEQ ID NO:22) | |
|---|---|
| MKNHLLFWGV LAVFIKAVHV KAQEDERIVL VDNKCKCARI TSRIIRSSED | 50 |
| PNEDIVERNI RIIVPLNNRE NISDPTSPLR TRFVYHLSDL CKKCDPTEVE | 100 |
| LDNQIVTATQ SNICDEDSAT ETCYTYDRNK CYTAVVPLVY GGETKMVETA | 150 |
| LTPDACYPD | 159 |

The following primers, retrofitted with the indicated restriction sites for further cloning, were designed for amplifying the human J-chain cDNA out of RT-PCR prepared materials from human Buffy coat cells:

| | |
|---|---|
| 5′- GAA TTC AGA AGA AGT GAA GTC | (SEQ ID NO:48) |
| EcoRI | |
| 3′- GTC GAC TAT GCA GTC AGC AAT GAC | (SEQ ID NO:49) |
| SalI | |

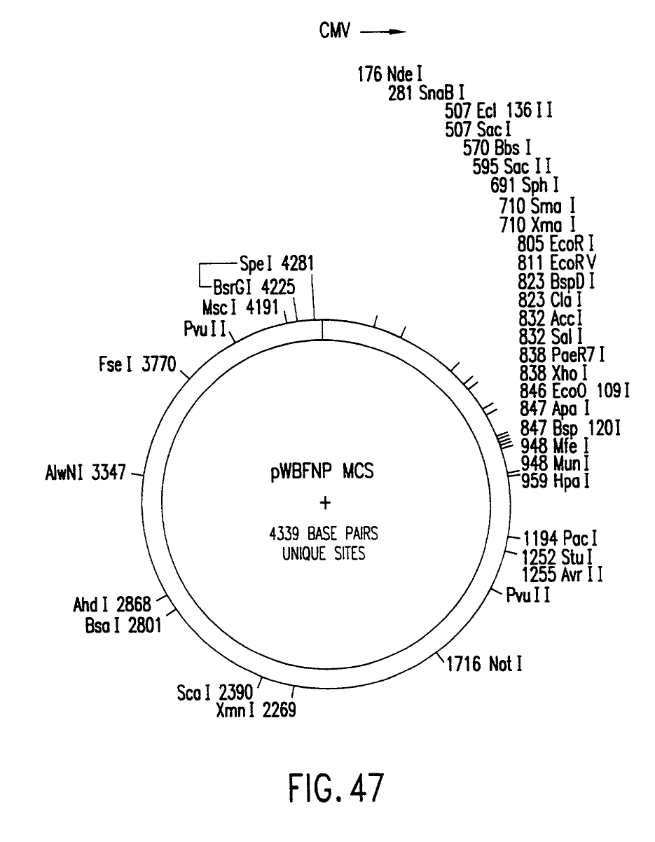

The J-chain cDNA and the 2.6.1 kappa gene isolated through RT-PCR were amplified using the above primers and a 500 base pair PCR product was isolated whose open reading frame encoded the 159 amino acid J-chain protein. The PCR product was cloned into the TA cloning kit (Invitrogen) and had an EcoRI restriction site on each end. This vector was digested with EcoRi and the digest cloned into pWBFNP MCS (FIG. 47) that was cut with EcoRI and treated with CIP. Orientation of the insert was determined through digestion with PvuII which created differently sized fragments based on orientation (PvuII sites were present in the pWBFNP MCS vector as shown in FIG. 47 and at position 421 in the J-chain insert. This vector was called pWBJ1

The 2.6.1 kappa chain was amplified by RT-PCR using the following primers:

| | |
|---|---|
| 5 prime: | |
| 5′ TGC AGG AAT CAG ACC CAG TC | (SEQ ID NO:50) |
| 3 prime: | |
| 5′ GTC AGG CTG GAA CTG AGG AGC A | (SEQ ID NO:51) |

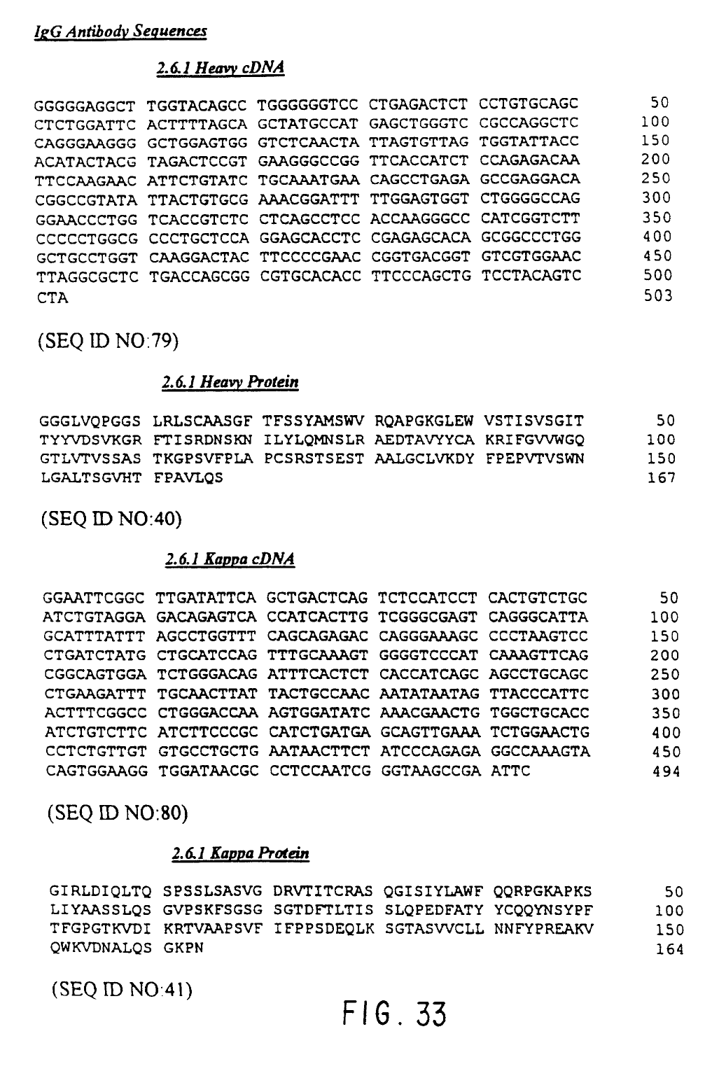

using the TA cloning kit providing EcoRi sites on each end of the VJCK insert. The kappa chain was sequenced. The kappa cDNA was EcoRI digested and cloned into the EcoRI site in pWBFNP MCS. Orientation was determined based on fragment size by NotI and PstI digestion of the NotI site in pWBFNP MCS and the PstI site contained at position 243 of the kappa insert shown in FIG. 33. This vector was called pWBK1.

In order to allow insertion of the J-chain expression cassette into pWBK1 from pWBJ1, pWBK1 was cut with Pacd and blunted and recut with AvrII and pWBJ1 was cut with SpeI and blunted and recut with AvrII and the blunted SpeI/AvrII fragment was cloned into pWBK1 blunt Pacl/AvrII to yield pWBK1(J). pWBK1(J) contained expression cassettes for both the 2.6.1 kappa chain and the J-chain.

pWBK1(J) was further modified to contain DHFR resistance through cloning DEFR through NotI digestion from a vector pWB DHFR (containing DHFR at NotI) into pWBK1(J) at the NotI site. This vector was called pWBK1(J) DHFR.

In order to make an IgG1 expression vector, the 2.6.1 heavy chain was amplified through RT-PCR using the TA cloning vector (Invitrogen) using the following primers:

| | |
|---|---|
| 5 prime: | |
| 5′ TCA TTT GGT GAT CAG CAC T | (SEQ ID NO:52) |
| 3 prime: | |
| 5′ GCT AGC TGA GGA GAC GGT GAC CAG G | (SEQ ID NO:53) |
| 3′ gamma 1 NheI (introduces a | |
| NheI restriction site) | |

The resulting product contained only the VDJ cDNA sequences and not the constant region. The sequence was confirmed by sequencing. This vector was utilized to prepare an IgG1 expression vector as described below.

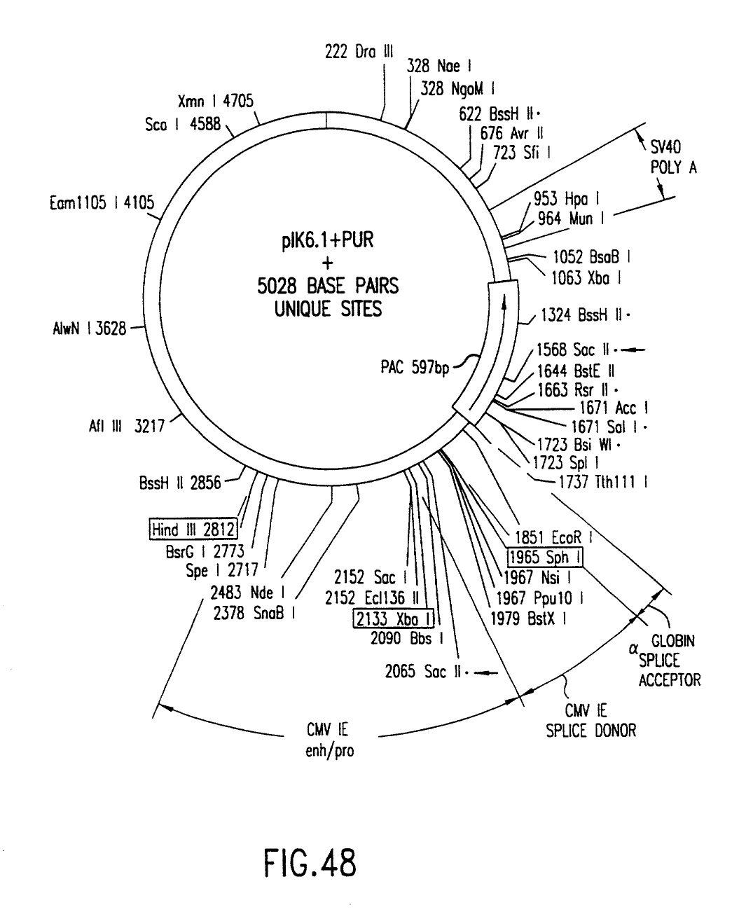

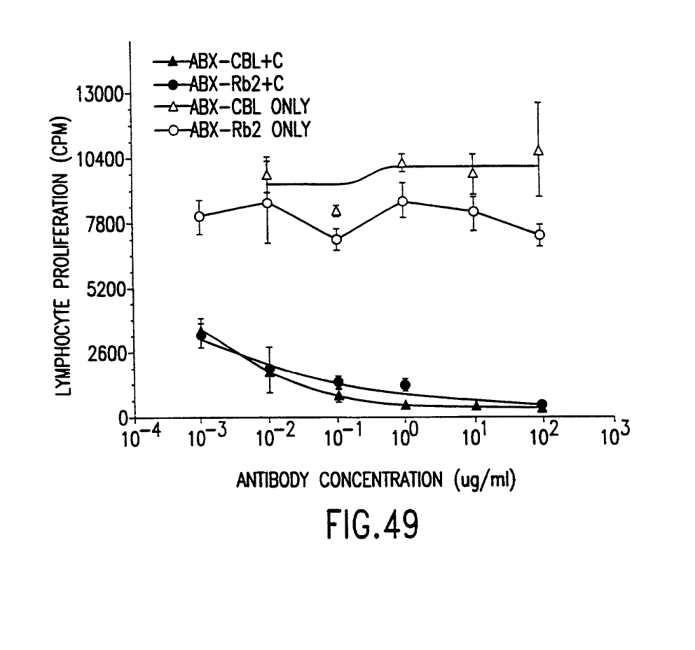

pWBFNP MCS was digested with EcoRI and treated with CIP and the EcoRI digest from the TA vector, above, was cloned into the vector. Orientation was determined by size through digestion with NheI, which confirmed the insertion, followed by digestion with Notl. This vector was called pWBVDJ261NheI. PWBVDJ261NheI was cut with XhoI and blunted and recut with NheI. A human gammal construct was cloned in from a pWBFNP vector containing the gammal constant region between NheI and EcoRI sites was cut with EcoRI and blunted and recut with NheI. This vector was called pWBVDJ261G1 (or pWBIgG1). A puromycin cassette was cloned in from a pIK6.1+puro vector (FIG. 48) which was cut with HindIII and blunted and recut with AvrII. The pWBIgG1 was cut with Pacd and blunted and recut with AvrII and the puro cassette was cloned therein. This vector was called pWBIgG1 Puro.

In order to make an IgM expression vector, the 2.6.1 heavy chain was amplified through RT-PCR using the TA cloning vector (Invitrogen) using the following primers:

| | |
|---|---|
| 5 prime: | |
| 5′ TCA TTT GGT GAT CAG CAC T | (SEQ ID NO:54) |
| 3 prime: | |
| 5′ GGA TCC TGA GGA GAC GGT GAC G | (SEQ ID NO:55) |
| 3′ Mu BamHI (introduces BamHI | |
| restriction site) | |

The resulting product contained only the VDJ cDNA sequences and not the constant region. The sequence was confirmed by sequencing. This vector was utilized to prepare an IgM expression vector as described below.

pWBFNP MCS was digested with EcoRI and treated with CIP and the EcoRI digest from the TA vector, above, was cloned into the vector. Orientation was determined by size through digestion with BamHI, which confirmed the insertion, followed by digestion with Noti. This vector was called pWBVDJ261BamHI.

A human Mu construct was PCR amplified from a yeast artificial chromosome construct, YAC 2CM, described in Mendez et al., (1997), supra. and U.S. patent application Ser. No. 08/759,620, filed Dec. 3, 1996, through RT-PCR using the TA cloning vector (Invitrogen) using the following primers:

| | |
|---|---|
| 5 prime: | |
| 5′GGA TTA GCA TCC GCC CCA ACC CTT | (SEQ ID NO:56) |
| (which introduced a BamHI | |
| restriction site on the | |
| 5′ end) | |
| 3′ prime: | |
| 5′ GTC GAC GCA CAC ACA GAG CGG CCA | (SEQ ID NO:57) |

The vector pWBVDJ261BamI was cut with BamHI and recut with XhoI. The TA cloning vector containing the Mu insert was cut with BarnmHl and XhoI (which is another site in the TA vector) and was cloned into the BamHI/XhoI sites of pWBVDJ261BamI. The resulting vector was called pWBVDJ261IgM (or pWBIgM). The vector was further equipped with a puromycin cassette in the same manner as described above in connection with the construction of pWBIgG1 Puro. The resulting vector was called pWBIgM Puro.

Experiment 17 Generation of Cell Line Expressing 2.6.1 IgG1 Antibodies

In order to generate a cell line expressing the 2.6.1 IgG1 antibody, we cotransfected DHFR⁻ CHO cells with The pWBIgG1 Puro vector and the pWBK1 DHFR vector through electroporation. This was accomplished by taking a stock of approximately 2×10⁷ DHFR CHO cells and electroporating at 290 V, 960 μFD, 200 μg of linearized plasmid DNA plus 200 μg of carrier DNA. Cells were seeded in α⁺ medium and allowed to grow for two days. 8×10⁵ cells were seeded in 10 cm dish in α⁻ medium with 4 μg/ml puromycin selection medium. Cells were incubated for 4-5 days and then transferred to α⁻ medium with 0.5 μM MTX at 5×10⁵ cells per 10 cm dish. Cells were incubated for approximately 14 days for selection and, thereafter, clones were picked and expanded and assayed for ability to bind to CD147 and the presence of IgG1.

We recovered a number of clones expressing a 2.6.1 antibody with a gamma-1 isotype that bound specifically to CD147.

Experiment 18 Generation of Cell Line Expressing 2.6.1 Multimeric IgM Antibodies

In order to generate a cell line expressing the 2.6.1 multimeric IgM antibody, we cotransfected DHFR⁻ CHO cells with The pWBIgM Puro vector and the pWBK1(J) DHFR vector through electroporation. The same techniques described in Experiment 18 were utilized.

We recovered a number of clones expressing a 2.6.1 antibody with a multimeric Mu isotype that bound specifically to CD147.

Experiment 19 Characterization of the 2.6.1 IgG1 and Multimeric IgM Antibodies

In order to assess the function of the 2.6.1 IgG1 and multimeric IgM antibodies, we assayed the antibodies in several assays. Each of the 2.6.1 IgG1 and multimeric IgM bound to CEM cells and bound to CD25⁺ activated human peripheral blood cells in a similar manner to the CBL1 and ABX-CBL. The antibodies were assayed in a potency and a lysis assay, in the same manner described above. In connection with these experiments, the 2.6.1 multimeric IgM antibody appeared approximately as active as CBL1 and ABX-CBL. Further, the 2.6.1 multimeric Igu antibody was capable of acting in ADCC as shown in FIG. 50.

Experiment 20 Affinity Measurement of the 2.6.1 Multimeric IgM Antibodies

We also examined the affinity of the the 2.6.1 multimeric IgM antibody in comparison to ABX-CBL and certain other forms of the 2.6.1 antibody. Affinity measurements were conducted as described in Mendez et al., (1997), supra. and U.S. patent application Ser. No. 08/759,620, filed Dec. 3, 1996. The results are shown in the following Table:

TABLE 2

| | | | | | | BIAcore |
|---|---|---|---|---|---|---|
| | | | | | | surface |
| | | On-rates | Off-rates | KA | KD | Hu rCD147- |
| Antibody | Ig class | ka (M⁻¹s⁻¹) | kd(s⁻¹) | kd/ka (M⁻¹) | ka/kd (M) | IgG [RU] |
| ABX-CBL | M IgM | 7..25 × 10⁵ | 3.76 × 10⁻⁴ | 1.39 × 10⁹ | 5.18 × 10⁻¹⁰ | 791 |
| ABX-CBL | M IgM | 6.34 × 10⁴ | 4.94 × 10⁻³ | 1.28 × 10⁷ | 7.84 × 10⁻⁸ | 791 |
| | monomer | | | | | |
| CEM | Hu IgG2 | 8.20 × 10⁵ | 3.75 × 10⁻⁴ | 2.19 × 10⁹ | 4.57 × 10⁻¹⁰ | 791 |
| 2.6.1 | | | | | | |
| CEM2.6.1 | Hu IgG2 | 7.17 × 10⁵ | 4.03 × 10⁻⁴ | 1.78 × 10⁹ | 5.61 × 10⁻¹⁰ | 242 |
| CEM2.6.1 | Hu IgM | 6.52 × 10⁵ | 2.03 × 10⁻⁴ | 3.21 × 10⁹ | 3.12 × 10⁻¹⁰ | 242 |
| CEM2.6.1 | Hu IgM | 2.63 × 10⁵ | 1.67 × 10⁻³ | 1.57 × 10⁸ | 6.39 × 10⁻⁹ | 242 |
| | monomer | | | | | |
| CEM2.6.1 | Hu IgG1 | 3.13 × 10⁵ | 2.01 × 10⁻⁴ | 1.55 × 10⁹ | 6.43 × 10⁻¹⁰ | 242 |

Experiment 21 Human Clinical Trial with ABX-CBL Antibody
Phase II Clinical Trial of ABX-CBL

A. Background

As we mentioned above, in view of the positive results observed with respect to the CBL1 antibody, we undertook clinical trials utilizing ABX-CBL. The first such trial was a Phase II, multicenter, open label, dose escalation clinical trial examining multiple intravenous infusions of four doses of ABX-CBL in patients with steroid resistant GVHD. The trial enrolled patients with acute GVHD who were unresponsive to at least three days of treatment with corticosteroids and who had a severity index of at least B according to a modified IBMTR Severity Index (Rowlings et al. “IBMTR severity index for grading acute graft-versus-host disease: retrospective comparison with glucksberg grade” British Journal of Haematology 97: 855-864 (1997)). In the trial, four different doses were administered intravenously in a dose escalation design using an induction regimen of seven days followed by a maintenance dose of twice weekly for two weeks. Patients were followed for 8 weeks after completion of the treatment course. Long-term safety follow-up has been instituted.

The study was designed with three primary objectives and four secondary objectives under review, as follows:

Primary Objectives (i) to assess the safety of multiple doses of ABX-CBL in patients with steroid resistant acute GVHD; (ii) to determine the maximum tolerated IV dose of ABX-CBL in patients with steroid resistant acute GVHD; and (iii) to determine the pharmacokinetics of multiple doses of ABX-CBL in patients with steroid resistant acute GVHD.

Secondary Objectives (i) to assess the clinical efficacy of four different doses of ABX-CBL in patients with steroid resistant acute GVHD; (ii) to assess a dose response of ABX-CBL; (iii) to assess long-term safety in patients with acute GVHD who have received multiple doses of ABX-CBL; and (iv) to assess the long-term survival in patients with acute GVHD who have received multiple doses of ABX-CBL.

Determination of the dosing for ABX-CBL was considered essential. As discussed above, the initial clinical trials conducted with the CBL1 antibody utilized ascites fluid that was not purified. Thus, the concentration of the antibody within the materials given to patients was not known. Further, because CBL1 was generated in a cell line that was not producing solely the IgM, but also an IgG, the concentration of the IgM antibody given to patients was even less clear.

In order to assess the above objectives, a four cohort trial plan was established with the following dose cohorts of patients:

Cohort 1: 0.01 mg/kg

Cohort 2: 0.1 mg/kg

Cohort 3: 0.3 mg/kg

Cohort 4: 1.0 mg/kg

Patients in all cohorts were to receive, and received, up to 11 intravenous infiusions of ABX-CBL. ABX-CBL was infused over 2 hours via a syringe pump. The dosing schedule was as follows: daily times 7 days, followed by twice a week for two weeks. Safety evaluations were conducted prior to advancing to the next dose cohort. 27 patients were enrolled across all 4 of the dose cohorts.

During the conduct of the study, adverse events were observed in patients in the third cohort, receiving 0.3 mg/kg of ABX-CBL. There, several of the patients experienced myalgia or myalgia-like symptoms. As a result, the third dose cohort (0.3 mg/kg) was determined as the maximum tolerated dose. Thus, the fourth dose cohort was reduced to a dosage of 0.2 mg/kg so that the actual dosing utilized in the study was as follows:

Cohort 1: 0.01 mg/kg

Cohort 2: 0.1 mg/kg

Cohort 3: 0.3 mg/kg

Cohort 4: 0.2 mg/kg

The eligibility requirements for patients to enter the study were as follows:
One year old or older
Stem Cell transplant within 100 days
Steroid resistant acute GVHD with a severity index of B, C, or D
No experimental drugs or devices within 30 days of enrollment unless mutually agreed upon by the investigator, the sponsor's medical monitor, and the FDA
ANC>500/mm³ with or without GCSF or GMCSF

Patients were screened and assigned to a treatment cohort once the patient met the eligibility criteria. Standard post stem cell transplant treatment was continued.

Once dosing was initiated, patients were infused with ABX-CBL the applicable dose for their dose cohort daily for 7 days (referred to as an induction regimen) followed by infusions 2 times per week for two weeks (referred to as a maintenance regimen). Patients were followed for 8 weeks following their infusions (visits are weekly for 4 weeks followed by a visit 4 weeks later) for safety and clinical effect. Further, patients who received at least one infusion of ABX-CBL were scheduled to participate in a long term follow up program to evaluate the long term safety of ABX-CBL and long term survival.

Safety was assessed by monitoring adverse events while on study as well as vital signs during the infusion of ABX-CBL. Further, patients received frequent physical exams and underwent extensive laboratory studies. Laboratory studies included complete blood counts, T-cell subsets, serum chemistries, and urinalyses at regular intervals as outlined below. Baseline CPK with isoenzymes were obtained on all patients and patients who experienced any infusion related adverse experiences were reanalyzed with CPK and isoenzymes. In addition, patients were monitored for Human Anti Mouse Antibody (HAMA) response by ELISA. Further, five patients in each cohort were assigned to have pharmacokinetic blood samples for pK profile.

Clinical effect of ABX-CBL was assessed by evaluating changes to the overall score of acute GVHD based upon the modified IBMTR Severity Index (Rowlings et al. “IBMTR severity index for grading acute graft-versus-host disease: retrospective comparison with glucksberg grade” British Journal of Haematology 97: 855-864 (1997)), time to response, duration of response, time and incidence of flare of acute GVHD, and length of hospitalization.

B. Protocol Procedures

In connection with the trial, the following tests, observation schedules, preparations of the study medication were utilized:

TABLE 3
TESTS AND OBSERVATION SCHEDULE

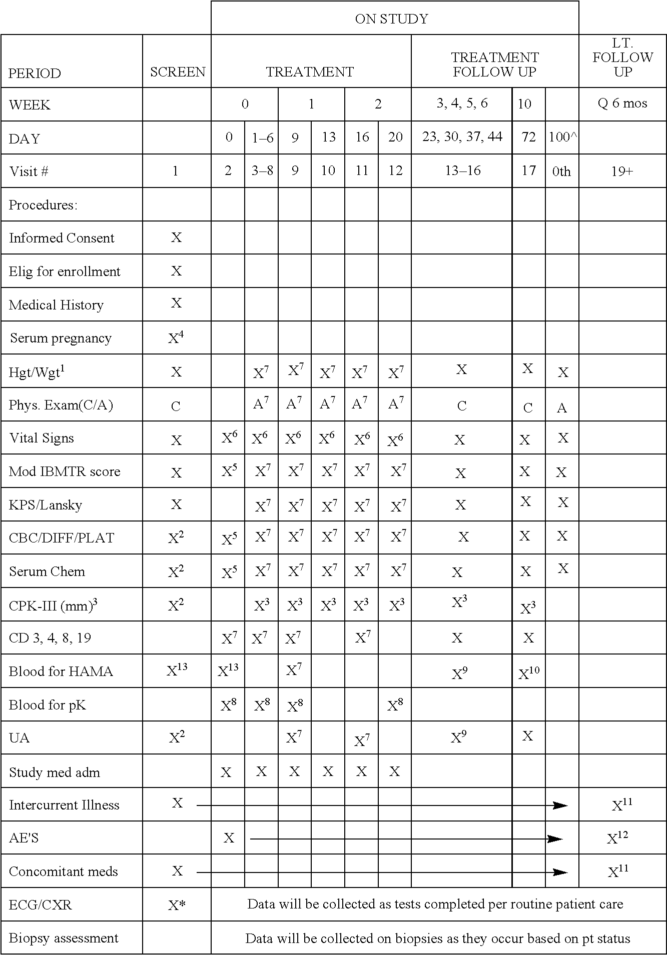

\hat{ }This visit occurs 100 days post allogeneic stem cell transplant not post infusion of study medication
*Obtain if not completed Within 7 days of randomization (ECG if patient is ≧ 16 years)
C/A C = Complete PE, A = Abbreviated PE
¹Obtain Height at first visit only
²Obtain within 48 hours of randomization request
³Obtain at baseline, obtain if patient experiences any infusion related AE's (refer to protocol)
⁴Serum pregnancy will be obtained on females of child bearing potential (refer to protocol)
⁵Obtain if results are >8 hours from the start of the infusion of the study medication
⁶Obtain vital signs (T, P, R, BP) prior to the start of the infusion (maximum of 10 mins), q 15 mins during the first hour of the infusion, followed by 90, 120, 180, 240, 300, and 360 minutes after the start of the infusion
⁷Obtain prior to the start of the infusion (maximum of 12 hours)
⁸Obtain just prior to the start of the first infusion and the following timepoints after the completion of the first infusion; 15 and 30 minutes, 1, 2, 4, 8, 12, 18, and 24 hours (before the 2^{nd} infusion) and at 4 hours after the completion of the Days 9 and 20 infusions (assigned patients only)
⁹Obtain at weeks 4 and 6 only
¹⁰Obtain during long term follow up if + at end of study
¹¹GVHD status and current treatment(s)
¹²Resolve any ongoing AEs
¹³Obtain HAMA during screen if the patient previously received a murine derived product. This needs to be negative in order for the patient to qualify. If the patient has never received a murine product, this is to be obtained on Day 0 prior to the start of the infusion

In connection with the trial, it was preferred that the modified IBMTR Severity Index scoring was completed by the same physician.

The following labs were to be completed by laboratory at each clinical site (local lab):

HEMATOLOGY
CBC w/ differential
White blood cells count
(WBC)
WBC differential (diff)
bands/stabs
neutrophils
EOS
basophils
lymphocytes
monocytes
Red blood cell count (RBC)
Hemoglobin (Hgb)
Hematocrit (Hct)
Platelet count (Plt)
URINALYSIS
Specific gravity
PH
Protein
Glucose
Ketones
SERUM CHEMISTRY
Sodium (Na)
Potassium (K)
Chloride (Cl)
Bicarbonate (HCO3)
Glucose
Blood Urea Nitrogen (BUN)
Creatinine (Cr)
Uric acid
Albumin
Total protein
Total bilirubin (bili)
Alkaline Phosphatase (alk phos)
Alanine aminotransferase (ALT, SGPT)
Aspartate aminotransferase (AST, SGOT)
Calcium (Ca)
Phosphate (PO4)
CPK-III isoenzyme* (mm) (skeletal muscle)
Females only:
serum Pregnancy
(if applicable)
*Obtain CPK with isoenzymes at baseline and post infusion on any patients with infusion related AE's.

In addition, the following lab assessments were completed by a central testing laboratory:
T cell subset (CD3, 4, 8) and CD19: lymphocyte count, %, and CD4:CD8 ratio)

Further, the following lab assessment were completed by Abgenix, Inc.:
ELISA for HAMA
pK (a minimum of 5 patients/cohort to include those who previously received a murine product)

The Study Medication (ABX-CBL) was prepared and administered as follows:

ABX-CBL is a protein so it requires gentle handling to avoid foaming. The avoidance of foaming during product handling, preparation, and administration is important because foaming can lead to denaturization of the protein product. The pharmacist prepared each dose of study medication. The dose was based upon the patient's weight prior to randomization and the patient's cohort assignment, therefore the patient will receive the same dose for all 11 infusions. The pharmacist prepared the syringe and filter (filter supplied by Abgenix) and sent this to the patient unit for patient dosing.

Infusion setup: The infusion syringe was prepared using aseptic techniques. The appropriate volume of study medication was drawn up into the syringe(s), followed by the calculated volume of the pyrogen-free 0.9% sodium chloride solution, USP (saline solution). A 0.22 micron low-protein binding filter was attached and the tubing was primed to minimize fluid loss and according to the manufacturer's instructions.

Infusion volume: The total infusion volume (study medication+saline solution) to be infused for each infusion (0.01, 0.1, 0.3, 0.2 mg/kg) is equal to the patient's weight in kg. Below are examples:

TABLE 4

| Pt's Weight | | Total Infusion Volume |
|---|---|---|
| (kg) | Cohort Assignment | (mL) |
| 70 kg | 0.01 | 70 mL |
| 70 kg | 0.1 | 70 mL |
| 70 kg | 0.3 | 70 mL |
| 70 kg | 0.2 | 70 mL |

The formula below was used to determine the volume of study medication and saline solution for each dose.
a. Dose required=patient's weight×mg/kg (mg/kg is based upon cohort assignment)
b. ABX-CBL Volume required=dose/study medication concentration (1 mg/mL)
c. Number of vials required=volume of ABX-CBL required (b above)/5 mL (each vial contains 5 mL of ABX-CBL)

d. Total volume to be administered: For all treatment cohorts the patients lo received a total volume which was equal to their weight in kg (a 15 kg patient will receive a total of 15 mL, a 70 kg will receive 70 mL, etc.)

TABLE 5
Example:
Patient weighs 70 kg and is assigned to receive 0.3 mg/kg

a. 70 kg × 0.3 mg/kg = 21 mg
b. 21 mg = 21 mL
c. 21 mL/5 mL per vial = 4.2 vials, therefore 5 vials are required
d. 70 ml (total volume) − 21 mL (study med volume) = 49 mL
(saline solution)

The labeled, filled infusion syringe was sent to the patient unit for infusion, making sure that all clamps on the infusion set were closed to prevent leakage of the study medication and/or normal saline. All caps were secured in place to maintain a closed system. The sponsor provided the label for the infusion syringe and this label will contain the following:

space to record the patient study ID and initial

space to record the date and time the study medication was prepared along with the expiration date and time

space to record the initials of the person who prepared the study medication and the infusion set

Infusion instructions:
“Caution: New Drug-Limited by Federal Law To Investigational Use”
Administer infusion over 2 hours via syringe pump
Do not mix with any other medication.*

Space to specify the infusion rate based upon the total volume. For a 70 mL volume, the infusion rate would be 35 mL/hour.

The person preparing the study medication was responsible for completing the above information on the label.

For the infusion, most patients had an indwelling central line, therefore a new catheter was not be required as long as there is a dedicated line for the infusion of ABX-CBL. During the administration of ABX-CBL no other medications were to be infused via the specific port or IV line. If a central line was not available, ABX-CBL could also be infused in a peripheral intravenous line. Because this was a trial, ABX-CBL was not mixed with other medications. If another medication was previously infused in the port, the lumen was flushed with 3-5 cc of normal saline (depending on the size catheter, lumen used, and patient's size) to clear any pre-existing medications from the line and the new infusion setup from the pharmacist was attached to the port or 3-way stopcock (not piggy backed onto another line) for infusion.

The protocol was composed of four study periods: screen, treatment, treatment follow up, and long term follow up.

1. Screen Period

The screen period began the day the patient or the patient's legal guardian signs the informed consent and ends at treatment assignment notification. Patients could be screened for enrollment into this study up to 100 days after stem cell transplant. Patients who failed to develop steroid-resistant acute GVHD were not enrolled into the study.

Each patient must understand and have signed an IRB approved informed consent form. If the patient was a minor, the patient's legal guardian was to sign the informed consent form.

The following procedures were to be completed after the informed consent form is signed but prior to requesting treatment assignment. The results of these procedures were not more than 8 hours old unless otherwise indicated. These procedures include:
a. Complete medical history
b. Complete physical examination, which includes weight (this is the weight to be used to determine the required dose of study medication throughout the study) and height
c. Vital signs (oral temperature, resting pulse, respiration, and blood pressure)
d. Medication history and stem cell transplant treatment history from 30 days prior to requesting treatment assignment
e. Modified IBMTR Severity Index for acute GVHD
f. Assessment of intercurrent illness(es)
g. Karnofsky Performance Scale (KPS) (age ≧16 years) or Lansky Scale (age <16 years)
h. The following lab results were obtained if not obtained within 48 hours prior to randomization:
   CBC with diff and platelets
   Serum Chemistry (refer to Appendix V)
   Baseline CPK-UIL isoenzyme (mm)
   Serum Pregnancy test. This may be waived for women who are not of child bearing potential or who, in the opinion of the investigator, are sterile due to the pre conditioning for the stem cell transplant
   Urinalysis
i. CXR if not completed within the previous 7 days
j. ECG if not completed within the previous 7 days for all patients 16 years of age or older.
k. Obtain serum specimen to be assayed by Abgenix for the determination of a positive HACA/HAMA for any patient who previously received a murine chimeric or fully murine product. This sample was to be shipped on dry ice overnight to Abgenix and results were generally available within 24 hours of Abgenix's receipt of the sample.

After the above were completed and the investigator determined that the patient was eligible for treatment, the clinical center requested (via fax) the cohort assignment from the sponsor. The clinical center generally received notification of the treatment assignment by fax within 3 hours of the request.

2. Treatment Period

The treatment period began when the clinical site received the patient's treatment assignment and ended when the patient completed the infusion regimen (11 doses). This period generally lasted a maximum of three weeks. The patient was considered “on study” once the patient was dosed and was considered “off study” after the completion of the week 10 visit procedures or when the patient withdrew from the study.

3. Week 0, day 0

Pre-Infilsion Procedures:

The pharmacist would prepare the study medication for infusion while the following visit procedures are being completed:
a. Update any changes in concomitant medications or intercurrent illnesses
b. Modified IBMTR Severity Index if the previous score was obtained greater than 8 hours prior to the start of the infusion
c. Blood draw for the following:
   CBC with diff and platelets (if previous results are >8 hours from the start of the infusion of study medication)
   Serum chemistry (if previous results are >8 hours from the start of the infusion of study medication)
   CD3,4, 8, & 19
   Baseline HAMA (patients who had blood drawn for HAMA as part of their eligibility screen procedure do not need to have this sample obtained)
   Baseline pK sample up to 10 minutes prior to the start of the infulsion (for assigned patients only)

Study Medication Infusion Procedures:

The pharmacist prepared the study medication such that the maximum total volume to be infused is dependent upon the patient's weight and cohort assignment (total volume of study medication and normal saline). The study medication was generally infused over 2 hours and the patient was closely monitored during the infusion and for the following 4 hours for any untoward reactions to the infusion. As of the start of the infusion of the study medication, the patient was monitored for adverse events on an ongoing basis. The sponsor was notified immediately of any suspected infusion related adverse experiences (cytokine release syndrome: fever, chills, rigors/shakes, hypotension, and rash or hypersensitivity reaction: fever, chills, bradycardia/cardiac arrest, respiratory arrest, acute respiratory distress syndrome, rash/urticaria, pancytopenia, increased liver transaminases, and arthralgias/myalgias). If an infusion reaction is suspected and the patient experiences myalgias or any muscular problems, CPK-III isoenzyme (mm) were obtained.

Infusion Vital Signs

During the infusion, vital signs (T, P, R. BP) were obtained just prior to the start of the infusion (a maximum of 10 minutes prior to the start of the infusion), every 15 minutes during the first hour of the infusion (4 sets), followed by 90 minutes after the start of the infusion, and at the completion of the infusion (120 minutes after the start of the infusion). Vital signs were generally obtained hourly for the next 4 hours (4 sets at 180, 240, 300, and 360 minutes after the start of the infusion). After the infusion vital signs have been completed, vital signs were monitored according to the established guidelines used by the clinical center.

Pharmacokinetic Blood Samples:

Blood for pK analysis was obtained from at least 5 patients in each cohort. All patients enrolled in study who previously received a murine product had pK assessments completed. Blood samples were generally obtained at the following times after the completion of the first infusion; 15 and 30 minutes, 1, 2, 4, 8, 12, 18, and 24 hours. The 24 hour post infusion sample was obtained prior to the start of the second infusion of ABX-CBL.

4. Week 0, days 1-6

The patient received a daily infusion of the study medication for 7 consecutive days (induction regimen). Each subsequent infusion generally began at the same time as the first infusion (±60 minutes). The dose was based upon the pre-enrollment weight, therefore, the patient will receive the same dose throughout the treatment period. Data was collected on any patients having an ECG or CXR completed at any time during the treatment period, otherwise routine ECGs and CXRs were not required. The same will hold true for any biopsies completed during this period.

The following procedures were generally completed within 12 hours prior to the start of each infusion unless otherwise noted:
a. Abbreviated physical exam (refer to Appendix II)
b. Weight
c. KPS or Lansky Scale
d Modified IBMTR Severity Index
e. Update any changes in concomitant medications or intercurrent illnesses
f. Adverse experience assessment
g. Blood draw for the following(refer to Appendix V for test to be processed by the local labs and those to be processed by the central lab):
   CBC with diff and platelets
   Serum chemistry
   CD3,4, 8, & 19
   Pharmacokinetic sample for assigned patients only and obtain prior to the start of the Day 1 infusion only (this is the 24 hour post infusion 1 sample).

Study Medication Infusion:

The study medication was infused over 2 hours following the above procedures. If an infusion reaction was suspected and the patient experiences myalgias or any muscular problems, CPK-III isoenzyme (mm) were obtained.

Infusion Vital Signs:

Vital signs (T, P, R, BP) were obtained according to the schedule described for the first infusion.

5. Week 1 (study days 9 and 13)

At the completion of the induction regimen, the patients were infused with the study medication twice a week for two weeks (maintenance regimen). The start time of each infusion in the maintenance regimen was generally ±60 minutes from the start time of the first infusion (Day 0). The following procedures were generally completed within 12 hours prior to the start of each infusion unless otherwise noted.
a. Abbreviated physical exam
b. Weight
c. KPS or Lansky Scale
d. Modified IBMTR Severity Index
e. Update any changes in concomitant medications or intercurrent illnesses
f. Adverse experience assessment
g. Urinalysis (study day 9 only)
h. Blood draw for the following(refer to Appendix V for test to be processed by the local labs and those to be processed by the central lab):
   CBC with diff and platelets
   Serum chemistry
   CD3, 4, 8, & 19 (day 9 only)
   HAMA (day 9 only)

Study Medication Infusion:

The study medication was infused over 2 hours and the procedures described above were again followed. If an infusion reaction was suspected and the patient experiences myalgias or any muscular problems, CPK-III isoenzyme (mm) were obtained.

Infusion Vital Signs:

Vital sign regimen described above was utilized.

Pharmacokinetic Sample:

A blood sample for pK analysis was obtained about 4 hours after the completion of the Day 9 infusion.

6. Week 2 (study days 16 and 20)

This was the second week of the maintenance regimen (dosing is twice a week for two consecutive weeks). The start time of each infusion was generally ±60 minutes from the start time of the Day 0 infusion. The following procedures were generally completed within 12 hours prior to the start of each infusion unless otherwise noted:
a. Abbreviated physical exam
b. Weight
c. KPS or Lansky
d. Modified IBMTR Severity Index
e. Update any changes in concomitant medications or intercurrent illnesses
f. Adverse experience assessment
g. Urinalysis (day 16 only)
h. Blood draw for the following(refer to Appendix V for test to be processed by the local labs and those to be processed by the central lab):
   CBC with diff and platelets
   Serum chemistry
   CD3, 4, 8, & 19 (day 16 only)

Study Medication Infusion:

The study medication was infused over 2 hours and the same procedures described above were followed. If an infusion reaction was suspected and the patient experiences myalgias or any muscular problems, CPK-III isoenzyme (mm) were obtained.

Infusion Vital Sigzns:

Vital sign regimen described above was utilized.

Pharmacokinetic Sample:

A blood sample for pK analysis was obtained about 4 hours after the completion of the Day 20 infusion.

7. Treatment Follow up Period (Weeks 3-10)

The treatment follow up period began after the completion of the Day 20 visit and ended at the completion of the week 10 visit. There were five visits during this period. When the patient completed the week 10 visit the patient was considered “off study”. If a patient is discharged from the clinical center during this study period, every attempt was made to complete a telephone assessment in place of an office visit. Weeks 3, 4, 5, 6, and 10 were treatment follow up visits. Safety, efficacy or signs of relapse was assessed at these visits. Patients who were partial or complete responders and have a flare of their GVHD were allowed to withdraw from the study and enroll into a separate open label, compassionate treatment protocol. Any biopsies, ECGs, and/or CXRs completed during the treatment follow up period were completed per routine patient care as specified at each clinical center, however, the data from these procedures was collected. Any patients who experienced a suspected infusion related adverse experience with myalgias or any muscular problems and who had elevated mm (isoenzyme which becomes elevated when there is muscular necrosis or inflammation) levels generally had a routine CPK-III (mm) sample obtained throughout the remainder of the study

8. Week 3 (Study Day 23)

The following procedures were completed at this visit:
a. Complete physical exam, vital signs, and weight
b. KPS or Lansky
c. Modified IBMTR Severity Index
d. Update any changes in concomitant medications or intercurrent illnesses
e. Adverse experience assessment
f. Hospitalization status (in patient or discharge)
g. Blood draw for the following:
   CBC with diff and platelets
   Serum chemistry (refer to Appendix V)
   CD 3, 4, 8, & 19

9. Week 4 (Study Day 30±1)

The following procedures were completed at this visit:
a. Complete physical exam, vital signs, and weight
b. KPS or Lansky
c. Modified IBMTR Severity Index
d. Update any changes in concomitant medications or intercurrent illnesses
e. Adverse experience assessment
f. Hospitalization status (in/outpatient or discharge from clinical center)
g. Urinalysis
h. Blood draw for the following:
   CBC with diff and platelets
   Serum chemistry (refer to Appendix V)
   CD 3, 4, 8, & 19
   HAMA

10. Week 5 (Study Day 37±1)

The following procedures were completed at this visit:
a. Complete physical exam, vital signs, and weight
b. KPS or Lansky
c Modified IBMTR Severity Index
d. Update any changes in concomitant medications or intercurrent illnesses
e. Adverse experience assessment
f. Hospitalization status (in/outpatient or discharge from clinical center)
g. Blood draw for the following:
   CBC with diff and platelets
   Serum chemistry (refer to Appendix V)
   CD 3, 4, 8, & 19

11. Week 6 (Study Day 44±1)

The following procedures were completed at this visit:
a. Complete physical exam, vital signs, and weight
b. KPS or Lansky
c. Modified IBMTR Severity Index
d. Update any changes in concomitant medications or intercurrent illnesses
e. Adverse experience assessment
f. Hospitalization status (in/outpatient or discharge from clinical center)
g. Urinalysis
h. Blood draw for the following:
   CBC with diff and platelets
   Serum chemistry (refer to Appendix V)
   CD 3, 4, 8, & 19
   HAMA

12. Week 10 (Study Day 72±2)

At the completion of this visit the patient was considered “off study”.

The following procedures were completed at this visit:
a. Complete physical exam, vital signs, and weight
b. KPS or Lansky
c. Modified IBMTR Severity Index
d. Update any changes in concomitant medications or intercurrent illnesses
e. Adverse experience assessment
f. Hospitalization status (in/outpatient or discharge from clinical center)
g. Urinalysis
h. Blood draw for the following:
   CBC with diff and platelets
   Serum chemistry (refer to Appendix V)
   CD 3, 4, 8, & 19
   HAMA (if any patient has a positive HAMA, blood draws for HAMA will be requested during the Long Term Follow up Period)

13. Additional Visit Timepoint (Day 100 post stem cell transplant)

Most patients were assessed 100 days post stem cell transplant. The order in which this visit occurs in relationship to the protocol visits varied on a patient by patient basis depending on when acute GVHD develops post stem cell transplant. Regardless of when day 100 occurs, the following procedures were completed at this visit (if the patient had been discharged from the clinical center every effort was made to obtain this information through a phone call to the patient and the patient's private physician):
a. Abbreviated physical exam, vital signs, and weight
b. KPS or Lansky
c. Modified IBMTR Severity Index
d. Update any changes in concomitant medications or intercurrent illnesses
e. Adverse experience assessment
f. Hospitalization status (in/outpatient or discharge from clinical center)
g. Blood draw for the following:
   CBC with diff and platelets
   Serum chemistry (refer to Appendix V)

14. Long Term Follow up Period

The long term follow up period begins the day after the completion of the week 10 visit and is planned to continue for 10 years or until the patient withdraws consent to be followed. The primary purpose of the long term follow up period is to determine long term safety of ABX-CBL and to determine the long term survival. The patient will be assessed every 6 months from their week 10 visit. These assessments will occur either by telephone interview or by office visit. Long term follow up data may be obtained by the sponsor, Abgenix, Inc., from the primary physician provided that the patient/legal guardian has provided written consent. All data will be entered into the database using the patient's unique study ID. The following information should be obtained during these phone calls or visits:
a. Determine the patient's assessment of their health status, this includes the closeout any AE's that were ongoing at the last “on study” visit
b. Determine the onset of any of the following:
   Death
   Opportunistic Infections
   Other immune impairments
   Other cancer(s)
   Congenital abnormality
   If female, if pregnant, status of baby (after pregnancy)
c. Determine if the patient is active in any other research (investigative products and/or devices) since the previous visit/call.

If the long term follow up visit data is obtained by the transplant team at the clinical center, a copy of each visit assessment will be faxed to the sponsor within 10 working days of the phone call/visit.

C. Determination of HAMA

This assay was designed to study the immunogenicity of ABX-CBL in human subjects to detect human antibodies against ABX-CBL (human anti-mCBL antibody) in human serum (human anti-murine antibody, HAMA, response).

Materials:
Negative Control, pool of HAMA negative sera (from Blood Centers of the Pacific, Irwin Blood Center, SF, Calif.) tested and pooled, stored at −20° C.
Positive Control, pool of HAMA positive sera (from immunizing XenoMouse mice (Abgenix, Inc.) with ABX-CBL and removal and pooling of serum), stored at −20° C.
ABX-CBL, 5 μg/50 μL (100 μg/nL), Abgenix, Lot No. 097-104-1, stored at −20° C. or equivalent
Biotinylated ABX-CBL (ABX-CBL-biotin), Abgenix, Lot No. J090-112 or equivalent
Streptavidin-HRP, Southern Biotechnology, Cat. No. 7100-05 or equivalent
O-phenylenediamine dihydrochloride (OPD) Substrate Tablets, 20 mg, Sigma, Cat. No. P-7288 or equivalent
O-phenylenediamine dihydrochloride (OPD) Substrate Tablets, 10 mg, Sigma, Cat. No. P-8287 or equivalent
Hydrogen Peroxide, 30%, Sigma, Cat. No. H-1009 or equivalent
Deionized, reverse osmosis purified water (DiH₂O) or equivalent

Coating ELISA Plate: Thaw a vial of ABX-CBL5 μg/50 μL (100 μg/mL) at room temperature for 2-5 minutes. Vortex on low speed for 3-5 seconds. Add 48 μL of ABX-CBL5 μg/50 μL (100 μg/mL) to 12 mL of Coating Buffer (NaHCO₃ at 16.8 gms/1.8 L DiWater to pH 9.6 w/5N NaOH)) in a 15 mL conical tube. Vortex the coating solution on low speed for 3-5 seconds. Pour the coating solution into a reagent reservoir. Using a multi-channel pipettor, add 100 μL of coating solution to each well. Cover plate with plastic plate sealer. Incubate plate at 2-8° C. for 16-24 hours. Wash the plates with 1× Wash Buffer (50 nL Tween 20 in 10 L10× PBS diluted by 10) using a plate washer. Using the multi-channel pipettor, add 100 μL of Blocking Buffer (20 gms BSA in 400 mL 10× PBS, 0.4 gms. Thimerosal, 4 mL Tween 20, diluted to 4 L DiWater) to each well. Cover plate with plate sealer and incubate for 1 hour at room temperature.

Preparation of Positive Control: Thaw 1 vial of positive control (HAMA positive serum) at room temperature for 10-20 minutes. Vortex positive control for 3-5 seconds on low speed. Avoid air bubbles. Add 20 μL of positive control to 180 μL of Blocking Buffer in a microcentrifuge tube. In well A1 and A2 of a low binding 96-well plate, add 20 μL of diluted positive control above to 180 μL of Blocking Buffer. Mix. Mix well by aspirating and dispensing the solution 5 times. Avoid air bubbles. Prepare 2 fold serial dilutions of the positive control. Note: Each plate should include the positive control in duplicate in columns 1 and 2. The following procedure is for one plate. Add 100 μL of Blocking Buffer to wells B3, B4 through H3, H4 on the plate as above. Using a multi-channel pipettor, transfer 100 μL of the solution in wells A1 and A2 to B1 and B2, respectively. Mix well by aspirating and dispensing 100 μL of the solution 5 times. Avoid bubbles. Transfer 100 μL of the solution from wells B1 and B2 to wells C1 and C2, respectively. Mix well by aspirating and dispensing 100 μL of the solution 5 times. Avoid bubbles. Continue dilutions down the plate from row to row with the last dilution in Row G (wells G1 and G2). Leave the Blocking Buffer in Row H as blank controls.

Preparation of Negative Control: Thaw negative control at room temperature for 20-30 minutes. Vortex the negative control for 3-5 seconds on low speed before transferring to the ELISA plate. Dilute negative control by adding 20 μL to 980 μL of blocking Buffer.

Preparation of Sample: Note 1: Serum samples should be prepared in a designated area. Note 2 Wear gloves when handling serum and follow Universal Precautions. Thaw serum samples at room temperature for 20-30 minutes. Vortex serum samples for 3-5 seconds on low speed. Dilute serum samples 1:50 by adding 20 μL of a serum sample to 980 μL Blocking Buffer in a titer tube. Mix the diluted samples by aspirating and dispensing 50 μL of the solution 5 times. Avoid bubbles. Wash the coated ELISA plate from Step 7.3.2 using a plate washer. Transfer 50 μL of positive control, negative control, samples and blank to the ELISA plate as above. Cover the ELISA plate with plastic plate sealer and incubate for two hours at room temperature. Shake the plate on low speed.

Preparation of ABX-CBL-biotin: Note: Minimum of 10 mL of diluted ABX-CBL-biotin is needed for each ELISA plate. Final dilution may be adjusted according to the potency of the reagent. Vortex ABX-CBL-biotin for 3-5 seconds on low speed. Dilute 15 μL of ABX-CBL-biotin into 1.4-85 mL of Blocking Buffer in a microcentrifuge tube. Total dilution is 1:100. Dilute 1200 μL of 1:100 diluted ABX-CBL-biotin into 10.80 mL of Blocking Buffer. Total dilution is 1:1000. Vortex for 3-5 seconds on low speed. Wash the coated ELISA plate using a plate washer. Using a multi-channel pipettor, add 100 μL of 1:1000 diluted ABX-CBL-biotin to each well of the ELISA plate. Cover the plate with plastic plate sealer and incubate for 1 hour at room temperature.

Preparation of Streptavidin-BPP: Note: Minimum of 10 mL of diluted Streptavidin-HRP is needed for each ELISA plate. Final dilution may be adjusted according to the potency of the reagent. Vortex Streptavidin-BRP for 3-5 seconds on low speed. Dilute 10 μL of Streptavidin-HRP into 990 μL of Blocking Buffer in a microcentrifuge tube. Total dilution is 1:100. Dilute 250 μL of 1:100 diluted Steptavidin-HRP into 12.25 mL of Blocking Buffer. Total dilution is 1:5000. Vortex for 3-5 seconds on low speed. Wash the ELISA plate from above using a plate washer. Using multi-channel pipettor, add 100 μL of 1:5,000 diluted Streptavidin-HRP to each well of the ELISA plate. Incubate the plate for 15 min at room temperature.

Preparation of Substrate Solution: Note 1: Minimum of 10 mL of Substrate Solution is needed for each ELISA plate. Note 2: Prepare Substrate Solution fresh prior to use. To make 12 mL of Substrate Solution, add one 10 mg OPD tablet, and 12 μL of 30% H₂O₂ into 12 mL of Substrate Buffer in a conical tube. Dissolve the tablet by leaving the tube at room temperature for 3-5 minutes. Vortex the solution for 3-5 seconds prior to adding to the plate. Wash the EI,ISA plate from above using a plate washer. Using a multi-channel pipettor, add 100 μL of Substrate Solution into each well and incubate for 15 minutes. Using a multi-channel pipettor, add 50 μL of Stop Solution (2 M H₂SO₄) to each well.

Reading ELISA plate(s): Set wavelength at, 492 nm and check automix function to premix plate for 5 seconds before reading plate. Use reduction function (Check L1) to subtract the calculated blank for the assay. Samples and controls are blanked against the buffer blank. Read plate using the SPECTRAmax 250 spectrophotometer within 30 minutes of stopping the assay.

As discussed above, the present assay was utilized for patient samples in connection with the present clinical trials and no patients tested positive for a HAMA response.

D. Determination of pK

The present assay was utilized in connection with pharmacokinetic (pK) studies to measure the presence of ABX-IL8 in human serum.

Materials:
ABX-CBL, anti-mouse CBL antibody, 5 μg/50 μL (100 μg/mL), Abgenix, Lot No 69-21-4 or equivalent
High, Medium and Low Positive Controls, ABX-CBL: 69-21-3, 69-21-2, 69-21-1 or equivalent
Goat anti-mouse IgM, Caltag, Cat. No. M31500, Lot No. 3501 or equivalent
Goat anti-mouse IGM-HRP, Caltag, Cat. No. M31507, Lot No. 2301 or equivalent
Normal human serum
O-phenylenediamine dihydrochloride (OPD) Substrate Tablets, 20 mg, Sigma, Cat. No. P-7288 or equivalent
O-phenylenediamine dihydrochloride (OPD) Substrate Tablets, 10 mg, Sigma, Cat. No. P-8287 or equivalent
Hydrogen Peroxide, 30%, Sigma, Cat. No. H-1009 or equivalent
Deionized, reverse osmosis purified water (DiH₂O) or equivalent
Buffers and solutions that are used herein are the same as the buffers and solutions described in connection with the HAMA assay unless described otherwise

Coating ELISA Plate: Note: Minimum of 10 mL of coating solution is needed for each ELISA plate. Pull vial of goat anti-mouse IgM (1 mg/mL) from the 2-8° C. refrigerator. Let stand for 2-5 minutes at room temperature. Vortex on low speed for 3-5 seconds. Add 3 μL goat anti-mouse IgM (1 mg/mL) to 15 mL of Coating Buffer in a 15 mL conical tube. Vortex the coating solution on low speed for 3-5 seconds. Pour the coating solution into a reagent reservoir. Using a multi-channel pipettor, add 100 μL of coating solution to each well. Cover the plate with a plastic plate sealer. Incubate at 2-8° C. for 16-24 hours. Wash the plate with 1× Wash Buffer using a plate washer.

Blocking ELISA Plate: Using the multi-channel pipettor, add 200 μL of Blocking Buffer to each well. Cover plate with plastic plate sealer and incubate for 1 hour at room temperature.

Preparation of Standard: Note 1: Blocking Buffer used in Sections 8.4 and 8.6 (except 8.4.4.1 and 8.6.3) contains 1% serum from untreated human subjects. Minimum of 9 mL of Blocking Buffer is needed for each plate. To make 10 mL of Blocking Buffer containing 1% serum, add 100 μL serum to 9.9 nL of Blocking Buffer in a conical tube. Vortex on low speed for 3-5 seconds. Thaw 1 vial of ABX-CBL standard (100 μg/mL) at room temperature for 10-20 minutes. Vortex 100 μg/nL ABX-CBL on low speed for 3-5 seconds. Avoid bubbles.

Initial Dilution of Standard: Using a single channel pipette, add 40 μL of 100 μg/mL stock to 360 μL of Blocking Buffer in a 1.7 mL microcentrifuge tube. Mix well. This is a 1:10 dilution equal to 10 μg/mL. Using a single channel pipette, add 40 μL of the previous 1:10 dilution (10 μg/mL) into 460 μL of Blocking Buffer in a 1.7 mL microcentrifuge tube. Mix well. This dilution is equal to a concentration of 800 ng/mL. Mix the diluted standard by vortexing on low speed for 3-5 seconds. Avoid bubbles. Prepare 2 fold serial dilutions of the standard. Note: Each blank low binding ELISA plate should include the standard in duplicate in columns 1 and 2. The following procedure is for one plate. Add 100 μL of Blocking Buffer to Wells B1, B2 through H1, H2. Transfer 200 μL of 800 ng/mL standard to Wells A1 and A2. Using a multi-channel pipette, transfer 100 μL of the solution in Wells A1 and A2 to Wells B1 and B2, respectively. Mix well by aspirating and dispensing 100 μL of the solution 5 times. Avoid bubbles. Transfer 100 μL of the solution from Wells B1 and B2 to Wells C1 and C2, respectively. Mix well by aspirating and dispensing 100 μL of the solution 5 times. Avoid bubbles. Continue dilutions down the plate from row to row with the last dilution in Wells H1 and H2.

Preparation of Positive Controls: Note: One vial of high, medium and low control is needed for each assay plate. Thaw 1 vial of high, medium and low controls at room temperature for 10-20 minutes. Vortex the controls for 3-5 seconds on low speed before transferring to the ELISA plate.

Preparation of Sample: Thaw serum samples at room temperature for 30 minutes. Vortex serum samples on low speed for 3-5 seconds prior to dilutions. Dilute serum samples 1:10 by adding 20 μL of a serum sample to 180 μL Blocking Buffer (without 1% serum) in Row A of a blank plate. Mix well by aspirating and dispensing 100 μL of the solution 5 times. Avoid bubbles. Prepare two fold serial dilutions of the sample. Using a multi-channel pipette, add 100 μL of Blocking Buffer to Row B through Row H. Transfer 100 μL of the diluted samples from Step 8.6.3 to Row B. Mix as above. Continue to transfer 100 μL of the samples from Row B to Row C, from Row C to Row D, and so on to Row H. Mix samples after each transfer by aspirating and dispensing 100 μL of the solution 5 times. Avoid bubbles. Wash the plate with 1× Wash Buffer using a plate washer. Transfer 50 μL diluted standard, controls and samples from blank plate to the ELISA plate. Start from Row H, then go to Row G and so on up to Row A. Check. plate template to add additional wells of buffer blank. Cover the plate with a plastic plate sealer and incubate for two hours at room temperature.

Prepare RP-conjugated detection antibody: Note: Minimum of 10 mL of diluted HRP-conjugated antibody is needed for each plate. Mix goat anti-mouse IgM-HRP by vortexing on low speed for 3-5 seconds. Dilute goat anti-mouse IgM-HRP to 1:1500 by adding 8 μL of goat anti-mouse IgM-BRP to 12 mL of Blocking Buffer in a 15 mL conical tube. Vortex. Wash the plate with 1× Wash Buffer using a plate washer. Using a multi-channel pipette, add 100 μL of diluted goat anti-mouse IgM-HRP (from Step 8.10.2) to each well of the plate. Cover the plate with a plastic plate sealer and incubate for 1 hour at room temperature.

Prepare Substrate Solution: Note 1: Minimum of 10 mL of Substrate Solution is needed for each plate Prepare Substrate Solution fresh prior to use. To make 12 mL of Substrate Solution, add one 10 mg OPD) tablet and 12 μL of 30% H₂O₂ to 12 mL of Substrate Buffer in a conical tube. Dissolve the tablet by leaving the tube at room temperature for 3-5 minutes. Vortex the solution for 3-5 seconds prior to adding to the plate. Wash the plate with 1× Wash Buffer using a plate washer. Using a multi-channel pipettor, add 100 μL of Substrate Solution into each well and incubate for 15 minutes.

Stopping ELISA reaction: Using a multi-channel pipette, add 50 μL of Stop Solution to each well.

Reading ELISA plate(s): Set wavelength at 492 nm and check automix function to premix plate for 5 seconds before reading plate. Use reduction function (check LI) to subtract the calculated blank for the assay. Standard, controls and samples are blanked against the buffer blank. Read plate(s) using the SPECTRAmax 250 or equivalent spectrophotometer within 30 minutes of stopping the assay, Operation and Maintenance of the Molecular Devices SPECTRAmax 250 Microplate Spectrophotometer.

Data Analysis: The OD for the standard is used to calculate the standard curve. Use “4-parameter fit” to curve fit the standard. Sample and control concentrations are calculated automatically by the software from the standard curve. The following criteria must be met in order for the assay to be valid: Only use OD's <4.0 for standard, controls and samples. Compare the results for the assay controls (High, Medium and Low). The values for the controls must fall within 20% of expected concentration and with coefficient of variation (CV) ≦20%. The CV of the standards between ST03 and ST06 must be ≦20%. The correlation coefficient of the standard curve of the assay must be ≧0.990.

The present assay was utilized for determining the pharmacokinetics of the ABX-CBL antibody in the present clinical trials. The results from our preliminary determinations of pKs in patients utilizing the above-assay are shown in FIG. 1.

E. Results

Herein, we describe the results that were observed in the treatment of patients with acute GVHD with ABX-CBL.

In the trial, twenty-seven patients were enrolled across the four dose levels. The lower doses were completed prior to enrolling in the higher dose cohorts. Patients who were treated at the higher dose in the original third cohort (0.3 mg/kg) experienced myalgia or myalgia-like symptoms. Abge:nix determined this dose to be the Maximum Tolerated Dose (MTD) and revised the last dose from 1.0 mg/kg to 0.2 mg/kg (mid dose between the MTD and the dose prior to the MTD).

Once the 4 dose cohorts were filled, additional patients were enrolled at a dose level of 0.15 mg/kg to 0.2 mg/kg. As of Jan. 13, 1999, a total of 44 patients (17 additional patients) have been enrolled. Data continues to be collected on these additional 17 patients. This data will be presented as it becomes available.

All data presented herein are based upon the initial 27 patients except for the Serious Adverse Event (SAE) Summaries. The SAE Summaries relate to all patients as of Jan. 13, 1999.

Patients had to receive a minimum of 4 infusions of ABX-CBL to be evaluated for efficacy. Of the twenty-seven patients enrolled, 23 met this criteria. Excluding the patients in cohort 1 (the no-effect dose). There was an overall response rate of 73% with a mean duration of 32 days.

Other than the incidence of myalgia, ABX-CBL was well tolerated. All patients were, and remain, negative for HAMA, and no reports of hypersensitivity to ABX-CBL have been received.

1. Demographics:

Of the twenty-seven patients enrolled, 21 were adults (age 16 or older) and 6 were pediatric (Table 4). Twenty-four patients were recipients of an allogeneic bone marrow transplant, and the other three received peripheral stem cells. The mean duration from the date of transplant to enrollment into this study was 48 days. Seven patients were entered into the study with an IBMTR grade of B, 10 with a grade of C and 10 with D. (Table 5). Table 6 lists the baseline score for the 23 patients evaluated for efficacy.

TABLE 6
GENDER/AGE CATEGORY

| | MALE | FEMALE | TOTAL |
|---|---|---|---|
| ADULT | 13 | 8 | 21 |
| PEDIATRIC | 4 | 2 | 6 |
| (<16 YRS) | | | |
| TOTAL | 17 | 10 | 27 |

TABLE 7
BASELINE IBMTR SEVERITY SCORE-ALL PATIENTS

| | B | C | D | |
|---|---|---|---|---|
| COHORT | n(%) | n(%) | n(%) | TOTAL N |
| 1 (0.01 mg/kg) | 2 (22%) | 3 (33%) | 4 (44%) | 9 |
| 2 (0.1 mg/kg) | 2 (29%) | 3 (42%) | 2 (29%) | 7 |
| 3 (0.3 mg/kg) | 1 (50%) | 1 (50%) | 0 | 2 |
| 4 (0.2 mg/kg) | 2 (22%) | 3 (33%) | 4 (44%) | 9 |
| TOTAL | 7 | 10 | 10 | 27 |

TABLE 8
BASELINE IBMTR SEVERITY SCORE FOR EVALUABLE PATIENTS

| | B | C | D | |
|---|---|---|---|---|
| COHORT | n (%) | n (%) | n (%) | TOTAL N |
| 1 (0.01 mg/kg) | 2 (25%) | 3 (38%) | 3 (38%) | 8 |
| 2 (0.1 mg/kg) | 1 (17%) | 3 (50%) | 2 (33%) | 6 |
| 3 (0.3 mg/kg) | 1 (50%) | 1 (50%) | 0 | 2 |
| 4 (0.2 mg/kg) | 2 (29%) | 2 (29%) | 3 (43%) | 7 |
| TOTAL | 6 | 9 | 8 | 23 |

2. Efficacy:

Patients eligible for enrollment into this study required a minimum IBMTR score of B. Patients who demonstrated at least a 2 index decrease in overall IBMTR score were considered responders. Those who decreased to no score, meaning there was no acute GvHD present, were considered to be complete responders. Only patients who received 4 or more infusions of ABX-CBL are included in the efficacy analyses. (Table 7)

TABLE 9
EFFICACY SUMMARY

| | EVALUATED | | |
|---|---|---|---|
| | FOR | RESPONDERS | MEAN DURATION |
| COHORT | EFFICACY (n) | n (%) | OF RESPONSE (n) |
| 1 (0.01 mg/kg) | 8 | 3 (38%) | 24 days (3) |
| 2 (0.1 mg/kg) | 6 | 4 (67%) | 11 days (3) |
| 3 (0.3 mg/kg) | 2 | 2 (100%) | 69 days (1) |
| 4 (0.2 mg/kg) | 7 | 4 (57%)* | 41 days (3) |
| TOTAL | 23 | 13 (57%) | 36 days |

*One patient responded to additional therapy with ABX-CBL in the ABX-CB-9702 protocol and is not included in the above table.

Overall, thirteen (57%) of the twenty-three patients demonstrated a response to ABX-CBL in ABX-CB-9701. The mean duration was 36 days. One additional patient who rolled over into protocol described below responded to additional therapy. This brings the overall response rate to 61%. The assumption going into the study was that the dose of 0.01 mg/kg would be the no effect dose. Assuming this dose to have no effect, the response rate was 73% (11 of 15 patients). With this assumption, the mean duration of response was 32 days.

The duration of response seems to increase as the dose is increased. One patient, [0108], was an outlier for duration in the first cohort. This patient's duration lasted at least 59 days. The duration may be longer, but the study ended at Day 72.

[Patient 0816] experienced severe myalgia at the 0.3 mg/kg dose level during the first infusion. This patient was continued at a decreased dose of 0.2 mg/kg for all subsequent infusions. Because of the change in dose, this patient is evaluated in the 0.2 mg/kg cohort for efficacy and in the 0.3 mg/kg for safety.

Only one patient in the lowest dose cohort and both patients in the highest dose level completed the study through Day 72. Four of the six patients in the 0.1 mg/kg dose group completed the study, and 4 of the 7 in the 0.2 mg/kg dose group completed. All patients who demonstrated a complete response also completed this study through Day 72.

3. Safety:

All patients who received any amount of ABX-CBL were evaluated for safety. ABX-CBL was well tolerated with the exception of myalgia, which became the Dose Limiting Toxicity (DLT). The incidence of myalgia increased in relationship to an increase in the dose administered. This led to the Maximum Tolerated Dose (MTD) at 0.3 mg/kg. The onset of the myalgia ranged from 20-60 minutes into the infusion and usually resolved within 1-2 hours afier the completion of the infusion. Of the 14 patients who experienced any grade of myalgia, two required being withdrawn from this study due to the myalgia. All myalgias resolved without sequelae except for one patient in whom myalgia persisted. This last incidence is under further evaluation and clarification. Table 6 summarizes the incidence of myalgia by severity and dose. Patients with adverse events listed as myalgia graded as “not related” or “unlikely” and with a baseline disease of myalgia are not included in the this table.

TABLE 10
INCIDENCE OF MYALGIA AND OUTCOME

| | 0.01 mg/kg | | 0.1 mg/kg | | 0.3 mg/kg | | 0.2 mg/kg | |
|---|---|---|---|---|---|---|---|---|
| | (n = 9) | | (n = 7) | | (n = 3) | | (n = 8) | |
| | | Study | | Study | | Study | | Study |
| SEVERITY | n (%) | status | n (%) | status | n (%) | status | n (%) | status |
| SEVERE | | | 1 | W/D | 1 | con't | 3 | con't |
| | | | | | 1 | dec. | 1 | W/D |
| | | | | | | dose | | |
| MODERATE | 2 | Con't | 1 | Con't | | | 1 | Con't |
| MILD | 1 | Con't | 1 | Con't | | | 1 | Con't |

W/D = withdrew from the study related to the myalgia

Abgenix continues to investigate the causality of myalgia and any possible inter-relationships. The following causes have been ruled out as a predisposing factor to those who do develop myalgia:

alteration in electrolytes

responders vs non responders

type of transplant

type of donor

steroid dose

Eleven Serious Adverse Experiences in eleven patients have been reported with ABX-CBL. Five “severe” events, all myalgia related, are listed as “probable” for the relationship to ABX-CBL. One event, “hepatic failure of unknown etiology” is listed as “suspected”. The remaining SAEs are listed as “unlikely” or “not related”.

Twenty-three of these events were evaluated as probably related to ABX-CBL and 7 as suspected. All other events were reported as “unlikely” or “not related”.

Of the 23 “probable” adverse events, all except 2 were myalgia related. One patient experienced moderate “fatigue” which resolved without sequelae. The other experienced moderate “hemolysis” which resolved with a sequelae of increased Liver Function Tests (LFT).

Of the seven events evaluated as “suspected” to be related to ABX-CBL, 1 event was severe, 4 were moderate, and 2 were mild in severity. All of these events resolved without sequelae. The severe event was “edema”. The four moderate events occurred in 4 patients and consisted of “moderate decrease in uric acid”, “fever/chills”, “hypotension”, and “fever”. The two mild events occurred in two patients and consisted of “low grade fever following study drug” and “chills”.

HAMA testing on all 27 patients has been negative through the patients' last study visit.

Lymphocyte counts were drawn from all patients just prior to the first infusion and at regular intervals throughout the study. Of the patients who enrolled into ABX-CB-9701, approximately 50% could not be evaluated on the basis of the immunocompromised state secondary to both BMT and their ongoing GvHD. Patients who are post stem cell transplant are immunodeficient secondary to their conditioning regimen as well as an exacerbation of their immunodeficient state from acute GvHD. To date, ABX-CBL does not appear to have an untoward effect on the T-cell counts.

Phase II Clinical Trial of ABX-CBL—Rescue Protocol

As patients completed the above-described Phase II trial, we also initiated a second Phase II continuation trial for such patients to continue to receive ABX-CBL for any flares of GVHD experienced. The continuation trial was designed as an open label clinical trial for patients with acute GVHD who have previous exposure to ABX-CBL. Those patients who had acute GVHD of grades II/I/IV severity, as discussed above, were eligible.

In the trial, all patients are receiving, or will receive, up to 7 intravenous doses (1^{st} treatment course) of ABX-CBL. The medication will be infised over 2 hours via a syringe pump for 7 consecutive days. The dose will be 0.2 mg/kg (approximate dose used effectively in clinical trial described above. If the first treatment course produced a therapeutic effect (complete or partial response), patients may receive a second treatment course prior to the onset of chronic GVHD, or day 200 post primary transplant whichever is reached first. The second treatment course with ABX-CBL will be handled on a case by case basis through a discussion with the medical monitor and the investigator.

The objectives of this trial were as follows:

To assess the safety of continued dosing with ABX-CBL in patients with acute GVHD.

To determine the clinical effect of repeat treatments of ABX-CBL in patients with flare of acute GVHD or patients who were previous treatment failures with ABX-CBL.

To allow treatment for patients who failed to demonstrate a clinical effect at a lower dose of ABX-CBL and/or to provide treatment for previous responders to ABX-CBL who are experiencing a flare of their acute GVHD.

To assess flare rates after initial treatment with AdBX-CBL.

All of the procedures described above in connection with the initial clinical trial were utilized in connection with this study, with only minor modifications.

Dosing, Dose Regimen, and Treatment with ABX-CBL

In view of the foregoing discussion and results, ABX-CBL provides a profound treatment for GVHD and likely other disease etiologies wherein lymphatic cells are deleteriously or undesirably activated. The results presented herein demonstrate that through administration of a dose of ABX-CBL greater than about 0.1 mg/kg and less than about 0.4 mg/kg of the antibody is efficacious in connection with the treatment of such disease etiologies. Preferably, the dose is from about 0.1 mg/kg to about 0.3 mg/kg and more preferably from about 0.15 mg/kg to about 0.2 mg/kg. Further, the dosing regimen disclosed herein of an induction regimen (plural daily infusions, herein daily for 7 days) followed by a maintenance regimen (periodic infusions, herein twice weekly for two weeks) appears to assist in remission of GVBD and certainly lessens the severity of patients' GVHD between flares of the disease.

As will be appreciated, both the purified ABX-C,BL, discussed in detail in the present invention and other anti-CD147 antibodies, such as those discussed herein, will be similarly efficacious.

In addition to GVHD, therapeutics in accordance with the present invention will likely be efficacious with respect to diseases having an etiology characterized by a harmful presence of activated T cells, B cells, or monocytes. As an example, GVHD is one such disease. However, many inflammatory diseases and autoimmune diseases can be characterized as sharing such an etiology. Further the therapies of the invention will likely be efficacious in the following disease etiologies, including, without limitation: graft versus host disease (GVHD), organ transplant rejection diseases (including, without limitation, renal transplant, ocular transplant, and others), cancers (including, without limitation, cancers of the blood (i.e., leukemias and lymphomas), pancreatic, and others), autoimmune diseases, inflammatory diseases (including without limitations arthritis, rheumatoid arthritis), and others.

Experiment 22 Surrogate Antibodies that Bind to Murine gp42 for Animal Models

As discussed above, certain animal models awe contemplated in connection ,with the present invention. One of the simplest animal models is the mouse. The 2.6.1 antibody did not bind to mouse gp42 (basigin or mouse CD147). Accordingly, we undertook the generation of anti-mouse gp42 antibodies from rats that could be utilized as a surrogate antibody to ABX-CBL and/or the 2.6.1 antibodies for use in such models. Described below is cloning strategy utilized to prepare fusion proteins for immunization of rats and the preliminary characterization of antibodies generated therefrom. The cloning strategy described below is further detailed in FIGS. 51 and 52.

Cloning of Hu-CD147IgG2 Fusion Protein

The following PCR primers were utilized, based on the CD147 sequence reported by Miyauchi et al. J. Biochem. 110:770-774 (1991) ( Gene Bank Accession # D45131)

5 prime:

(SEQ ID NO:58)

5′-GACTACGAATTCGGACCGGCGAGGAATAGGAATCATG-3′
and
3 prime:

(SEQ ID NO:59)

5′-GGATGGTGTTGGTAGCTAGCACGCGGAGCGTGATGATGGCCTG-3′

A 626bp PCR product was amplified from CD147/pBKCMV plasmid DNA template that encoded the amino terminal 202 amino acid residues of the extracellular domain of CD147.The PCR product was digested with EcoR1 and Nhe1 and ligated into pIK1.1Hu-CD4IgG2 expression vector digested with EcoR1 and Nhe1. The resulting construct, pIKHu-CD147IgG2 encodes a fusion protein consisting of the N-terminal 202 amino acids of CD147 the last four C-terminal residues of the extracellular domain of CD4 in frame with the hinge CH2 and CH3 domains of Hu IgG2.

Cloning of Mu-GP42IgG2 Fusion Protein

The following PCR primers were utilized, based on the GP42 sequence reported by Kanekura et al. Cell Struct. Funct. 16:23-30 (1991) (Gene Bank Accession # Y16256):

5 prime:

(SEQ ID NO:60)

5′-GACTACGAATTCACGAGGCGACATGGCGGCGGC-3′
and
3 prime:

(SEQ ID NO:61)

5′-GGATGGTGTTGGTAGCTAGCACACGCAGTGAGATGGTTTCCCG-3′

A 659bp PCR product was amplified from mouse lymph node cDNA and encodes the amino terminal 206 amino acid residues of the extracellular domain of GP42. The PCR product was digested with EcoR1 and Nhe1 and ligated into pIK1.1Hu- CD4IgG2 expression vector digested with EcoR1 and Nhe1 to create pIKMu-GP42 IgG2.

Stable CHO Cell Line Engineering

The EcoR1/Bgl2 fragments from pIK Hu-CD147IgG2 and pIKMu-GP42IgG2 were cloned into the expression vector pWBFNP DHBR digested with EcoR1/Bgl2. PWBFNP DBFR is a derivative of pWBFNP into which a DHFR cDNA under the transcriptional control of SV40 promoter/enhancer and SV40 poly A is cloned at the Notl site. The resulting constructs, Hu-CD147IgG2 DHFR and Mu-GP42IgG2 DHFR were introduced into DHFR deficient CHO cell lines by CaPo₄ mediated transfection. Stable lines were selected for their ability to grow in the absence of exogenous thymidine,glycine and purines. Clones secreting elevated levels of fusion proteins as judged by SDS-PAGE were suspension adapted to spinner flasks in serum-free media. Mu-GP42IgG2 and Hu-CD147IgG2 fusion proteins were purified from culture media by protein A chromatography.

Following generation of the fiusion proteins, rats were immunized using conventional techniques and hybridomas generated also using conventional techniques. Antibodies secreted by such hybridonias could then be utilized as surrogate antibodies in certain animal models, particularly, murine models.

### Incorporation by Reference

All references cited herein, including patents, patent applications, papers, text books, and the like, and the references cited therein, to the extent that they are not already, are hereby incorporated herein by reference in their entirety.

### Equivalents

The foregoing description, Figures, and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. An isolated human monoclonal antibody having an isotype that fixes complement and a variable region that binds to the epitope on CD147 bound by the IgM monoclonal antibody ABX-CBL, with the proviso that the antibody is not CBL1 (ATCC HB 8214).

2. The antibody of claim 1, wherein the antibody in the presence of complement acts to selectively kill cells selected from the group consisting of activated T-cells, activated B-cells, and monocytes but is substantially non-toxic to resting T-cells and resting B-cells.

3. canceled

4. The antibody of claim 1, wherein the isotype is selected from the group consisting of murine IgM, murine IgG2a, murine IgG2b, murine IgG3, human IgM, human IgG1, and human IgG3.

5. canceled

6. The antibody of claim 2, wherein the isotype is selected from the group consisting of murine IgM, murine IgG2a, murine IgG2b, murine IgG3, human IgM, human IgG1, and human IgG3.

7. An isolated human monoclonal antibody having an isotype that fixes complement and a variable region that binds to CD147 on populations of activated T-cells, activated B-cells, and resting or activated monocytes, that, in the presence of complement, selectively depletes such populations through complement mediated killing while being substantially nontoxic to other cells, with the proviso that the antibody is not CBL1 (ATCC HB 8214).

8. canceled

9. The antibody of claim 7, wherein the isotype is selected from the group consisting of murine IgM, murine IgG2a, murine IgG2b, murine IgG3, human IgM, human IgG1, and human IgG3.

10. An isolated human monoclonal antibody having the following characteristics:
(a) binds to CD147;
(b) shows a binding against CEM cell lysates on Western blot similar to that provided in FIG. 1;
(c) an isotype selected from the group consisting of murine IgM, murine IgG2a, murine IgG2b, murine IgG3, human IgM, human IgG1, and human IgG3;
(d) competes with ABX-CBL for binding to CD147;
(e) cross reacts with hn-RNP-k protein;
(f) binds to a consensus sequence on CD147 comprising RVRS (SEQ ID NO:106);
(g) selectively kills activated T-cells, activated B-cells, and monocytes in an MLR assay only in the presence of complement; and
(h) is substantially non-toxic to cells expressing CD55 and CD59, with and without the presence of complement,
with the proviso that the antibody is not CBL1 (ATCC HB 8124).

11. A method to select an anti-CD147 antibodies for the treatment of disease, comprising:
generating antibodies that bind to CD147 and that are capable of binding complement;
assaying the antibodies for one or more of the following properties:
(a) competition with ABX-CBL for binding to CD147;
(b) capability to selectively kill activated T-cells, activated B-cells, and monocytes in a MLR assay only in the presence of complement; and
(c) being substantially non-toxic to cells expressing CD55 and CD59, with and without the presence of complement,
with the proviso that the antibody is not CBL1.

12. The method of claim 11, further comprising the following property:
(d) binding to CEM cell lysates on Western blot in a manner similar to that provided in FIG. 1.

13. The method of claim 11, further comprising the following property:
(e) binding to a consensus sequence in a peptide of RXRS (SEQ ID NO:11).

14. The method of claim 11, further comprising the following property:
(f) cross reacts with hn-RNP-k protein.

15. The method of claim 11, further comprising the following property:
(g) binding to a form of CD147 expressed by COS cells and E. coli cells.

16. A method to treat disease, comprising providing an antibody that has an isotype that fixes complement and a variable region that binds to CD147 on populations of activated T-cells, activated B-cells, and resting or activated monocytes, that, in the presence of complement, selectively depletes such populations through complement mediated killing while being substantially nontoxic to other cells, with the proviso that the antibody is not CBL1.

17. The method of claim 16, wherein the antibody is a human antibody.

18. The method of claim 16, wherein the isotype is selected from the group consisting of murine IgM, murine IgG2a, murine IgG2b, murine IgG3, human IgM, human IgG1, and human IgG3.

19. A method to treat GVHD, comprising providing an antibody that has an isotype that fixes complement and a variable region that binds to CD147 on populations of activated T-cells, activated B-cells, and resting or activated monocytes, that, in the presence of complement, selectively depletes such populations through complement mediated killing while being substantially nontoxic to other cells, with the proviso that the antibody is not CBL1.

20. The method of claim 19, wherein the antibody is a human antibody.

21. The method of claim 19, wherein the isotype is selected from the group consisting of murine IgM, murine IgG2a, murine IgG2b, murine IgG3, human IgM, human IgG1, and human IgG3.

22. A human monoclonal antibody that binds to an epitope on CD147 comprising the consensus sequence RVRSH (SEQ ID NO:107), wherein the antibody is not CBL1 (ATCC HB 8214).

23. canceled

24. An isolated peptide comprising the sequence selected from the group consisting of RXRS (SEQ ID NO:11, RXRSH (SEQ ID NO:13 , RVRS (SEQ ID NO:106), and RVRSH (SEQ ID NO:107).

25. A method of producing an antibody that binds to the peptide of claim 24 comprising the steps of immunizing an animal with the peptide and collecting the antibody.

26. A human monoclonal antibody that binds to CD147.

27. A kit for the treatment of diseases having an etiology characterized by a harmful presence of activated T cells, B cells, or monocytes, comprising:
(a) a liquid preparation comprising an amount of an anti-CD147 antibody in a pharmaceutically acceptable carrier and
(b) instructions on administering said preparation to a patient suffering from a disease having the etiology characterized by a harmful presence of activated T cells, B cells, or monocytes to provide a dosage in the range of from about 0.1 mg/kg to about 0.3 mg/kg of the antibody.

28. The kit of claim 27, wherein the antibody comprises ABX-CBL.

29. The kit of claim 27, wherein the instructions further include instructions for the administration of the antibody in a series of administrations to provide a dosage in the range of from about 0.1 mg/kg to about 0.3 mg/kg of the antibody in each administration.

30. The kit of claim 27, wherein the disease comprises GVHD.

31. An article of manufacture for use in the treatment of diseases having an etiology characterized by a harmful presence of activated T cells, B cells, or monocytes, comprising:
(a) a sterile vial;
(b) an anti-CD147 monoclonal antibody in a pharmaceutically acceptable carrier contained within the vial; and
(c) instructions for administration of the antibody to a patient suffering from such a disease in a manner to provide a dosage in the range of from about 0.1 mg/kg to about 0.3 mg/kg of the antibody in each administration.

32. The article of claim 31, wherein the antibody comprises ABX-CBL.

33. The article of claim 31, wherein the instructions further include instructions for the administration of the antibody in a series of administrations to provide a dosage in the range of from about 0.1 mg/kg to about 0.3 mg/kg of the antibody in each administration.

34. The kit of claim 31, wherein the disease comprises GVHD.

35. A kit for the treatment of diseases having an etiology characterized by a harmful presence of activated T cells, B cells, or monocytes, comprising:
(a) a liquid preparation comprising an amount of an anti-CD147 antibody designated ABX-CBL in a pharmaceutically acceptable carrier and
(b) instructions on administering said preparation to a patient suffering from a disease having the etiology characterized by a harmful presence of activated T cells, B cells, or monocytes in a series of administrations to provide a dosage in the range of from about 0.1 mg/kg to about 0.3 mg/kg of the antibody in each administration.

36. The kit of claim 35, wherein the instructions further include instructions for the administration of the antibody in a series of administrations to provide a dosage in the range of from about 0.1 mg/kg to about 0.3 mg/kg of the antibody in each administration.

37. The kit of claim 35, wherein the disease comprises GVHD.

38. An article of manufacture for use in the treatment of diseases having an etiology characterized by a harmful presence of activated T cells, B cells, or monocytes, comprising:
(a) a sterile vial;
(b) an anti-CD147 monoclonal antibody designated ABX-CBL in a pharmaceutically acceptable carrier contained within the vial; and
(c) instructions for administration of the antibody to a patient suffering from such a disease in a manner to provide a dosage in the range of from about 0.1 mg/kg to about 0.3 mg/kg of the antibody in each administration.

39. The article of claim 38, wherein the instructions further include instructions for the administration of the antibody in a series of administrations to provide a dosage in the range of from about 0.1 mg/kg to about 0.3 mg/kg of the antibody in each administration.

40. The article of claim 38, wherein the disease comprises GVHD.

41. A kit for the treatment GVHD, comprising:
(a) a liquid preparation comprising an amount of an anti-CD147 antibody in a pharmaceutically acceptable carrier and
(b) instructions on administering said preparation to a patient suffering from GVHD in a series of administrations to provide a dosage in the range of from about 0.1 mg/kg to about 0.3 mg/kg of the antibody in each administration.

42. The kit of claim 41, wherein the antibody comprises ABX-CBL.

43. The kit of claim 41, wherein the instructions further include instructions for the administration of the antibody in a series of administrations to provide a dosage in the range of from about 0.1 mg/kg to about 0.3 mg/kg of the antibody in each administration.

44. An article of manufacture for use in the treatment of GVHD, comprising:
(a) a sterile vial;
(b) an anti-CD147 monoclonal antibody in a pharmaceutically acceptable carrier contained within the vial; and
(c) instructions for administration of the antibody to a patient suffering from GVHD in a manner to provide a dosage in the range of from about 0.1 mg/kg to about 0.3 mg/kg of the antibody in each administration.

45. The article of claim 44, wherein the antibody comprises ABX-CBL.

46. The article of claim 44, wherein the instructions further include instructions for the administration of the antibody in a series of administrations to provide a dosage in the range of from about 0.1 mg/kg to about 0.3 mg/kg of the antibody in each administration.

47. A kit for the treatment of GVHD, comprising:
(a) a liquid preparation comprising an amount of an anti-CD147 antibody designated ABX-CBL in a pharmaceutically acceptable carrier and
(b) instructions on administering said preparation to a patient suffering from GVHD in a series of administrations to provide a dosage in the range of from about 0.1 mg/kg to about 0.3 mg/kg of the antibody in each administration.

48. The kit of claim 47, wherein the instructions further include instructions for the administration of the antibody in a series of administrations to provide a dosage in the range of from about 0.1 mg/kg to about 0.3 mg/kg of the antibody in each administration.

49. An article of manufacture for use in the treatment of GVHD, comprising:
(a) a sterile vial;
(b) an anti-CD147 monoclonal antibody designated ABX-CBL in a pharmaceutically acceptable carrier contained within the vial; and
(c) instructions for administration of the antibody to a patient suffering from GVHD in a manner to provide a dosage in the range of from about 0.1 mg/kg to about 0.3 mg/kg of the antibody in each administration.

50. The article of claim 49, wherein the instructions firther include instructions for the administration of the antibody in a series of administrations to provide a dosage in the range of from about 0.1 mg/kg to about 0.3 mg/kg of the antibody in each administration.

51. A pharmaceutical composition, comprising an anti-CD147 monoclonal antibody designated ABX-CBL in a pharmaceutically acceptable diluent, buffer, or excipient.

52. The pharmaceutical composition of claim 51, wherein the antibody is provided in a dosage from about 0.1 mg/kg and about 0.2 mg/kg.

53. A method for the treatment of diseases having an etiology characterized by a harmful presence of activated T cells, B cells, or monocytes, comprising administering a liquid preparation comprising an amount of an anti-CD147 antibody in a pharmaceutically acceptable carrier to a patient suffering from such disease.

54. The method of claim 53, wherein the antibody comprises ABX-CBL.

55. The method of claim 53, wherein the administration is conducted to provide a dosage in the range of from about 0.1 mg/kg to about 0.3 mg/kg of the antibody in each administration.

56. The method of claim 53, wherein the disease comprises GVHD.

57. A method for the treatment of GVHD comprising administering a liquid preparation comprising an amount of an anti-CD147 antibody in a pharmaceutically acceptable carrier to a patient suffering from GVHD.

58. The method of claim 57, wherein the antibody comprises ABX-CBL.

59. The method of claim 57, wherein the administration is conducted to provide a dosage in the range of from about 0.1 mg/kg to about 0.3 mg/kg of the antibody in each administration.

60. The human antibody according to claim 26, wherein the heavy chain has an amino acid sequence selected from the group consisting of: SEQ ID NO:23; SEQ ID NO:25; SEQ ID NO:27; SEQ ID NO:29; SEQ ID NO:31; SEQ ID NO:33; SEQ ID NO:35; SEQ ID NO:37; SEQ ID NO:39; SEQ ID NO:40.

61. The human antibody according to claim 26, wherein the light chain has an amino acid sequence selected from the group consisting of: SEQ ID NO:24; SEQ ID NO:26; SEQ ID NO:28; SEQ ID NO:30; SEQ ID NO:32; SEQ ID NO: 34; SEQ ID NO:36; SEQ ID NO:38; and SEQ ID NO:41.
